(19) United States
(12) Patent Application Publication
(10) Pub. No.: US 2023/0366042 A1
(43) Pub. Date: Nov. 16, 2023

(54) DNA NANODEVICE HINGE BIOSENSORS AND METHODS OF USE THEREOF

(71) Applicant: Ohio State Innovation Foundation, Columbus, OH (US)

(72) Inventors: Patrick HALLEY, Columbus, OH (US); Carlos CASTRO, Powell, OH (US); Christopher LUCAS, Columbus, OH (US); Melika SHAHHOSSEINI, Columbus, OH (US); Alexander MARRAS, Powell, OH (US)

(21) Appl. No.: 18/029,770

(22) PCT Filed: Oct. 1, 2021
(86) PCT No.: PCT/US2021/053204

§ 371 Date: Mar. 31, 2023

Related U.S. Application Data
(60) Provisional application No. 63/086,644, filed on Oct. 2, 2020.

(51) Int. Cl. C12Q 1/70; C12Q 1/6818

(52) U.S. Cl. CPC C12Q 1/701; C12Q 1/6818

(57) ABSTRACT

The present disclosure relates to biosensors and methods of use thereof.

### CROSS-REFERENCE TO RELATED APPLICATIONS

This application claims the benefit of U.S. Provisional Application No. 63/086,644, filed Oct. 2, 2020, which is expressly incorporated herein by reference in its entirety.

### FIELD

The present disclosure relates to biosensors and methods of use thereof.

### BACKGROUND

The most common methods for testing patients suspected to be infected by a pathogen include real-time reverse-transcriptase polymerase chain reaction (RT-PCR) assays, lateral flow antigen detection and serological tests for antibodies. While the RT-PCR assay is accurate, the assay is time consuming and labor intensive, whereas antigen detection is limited in its sensitivity. In addition, serological tests fail to provide an accurate diagnosis in patients during an early phase of the infection or in cases involving immunodeficient individual. What is needed are systems and methods for accurate and fast detection of an infection or a disorder. The biosensors and methods disclosed herein address these and other needs.

### SUMMARY

In some aspects, disclosed herein is a biosensor comprising:
a DNA origami hinge that comprises one or more scaffold strands, one or more staple strands, and one or more overhang staple strands; and
a latch strand that is capable of hybridizing to the one or more overhang staple strands and a target nucleic acid, and wherein the latch strand comprises a toehold domain.

In some embodiments, each of the one or more overhang staple strands comprises one or more fastening sequences. In some embodiments, the DNA origami hinge is in a closed configuration when the latch strand is hybridized to the one or more fastening sequences. In some embodiments, the latch strand comprises at least 3 nucleotides complementary to each of the one or more fastening sequences. In some embodiments, the toehold domain does not hybridize to the fastening sequences.

In some embodiments, the latch strand has a higher binding affinity to the target nucleic acid than to the one or more fastening sequences.

In some embodiments, the toehold domain comprises a sequence complementary to the target nucleic acid. In some embodiments, the target nucleic acid displaces the one or more fastening sequences when hybridizing to the latch strand.

In some embodiments, the DNA origami hinge is in an open configuration when the latch strand is not hybridized to the fastening sequences.

In some embodiments, the latch strand comprises a sequence at least 80% identical to SEQ ID NO: 62, 64, 66, 303, 304, 305, 306, 307, or 308.

In some embodiments, the toehold domain comprises a sequence at least 80% identical to SEQ ID NO: 63, 65, 67, or 318.

In some embodiments, the one or more overhang staple strands comprises one or more sequences at least 80% identical to SEQ ID NOs: 2-61 or 310-317.

In some embodiments, the target nucleic acid is a single stranded nucleic acid.

In some embodiments, the target nucleic acid is a viral RNA. In some embodiments, the viral RNA is a SARS-COV-2 RNA. In some embodiments, the RNA virus comprises an influenza virus, HIV, hepatitis C virus, Ebola virus, rabies virus, or Dengue virus. In some embodiments, the influenza virus is influenza A virus or influenza B virus.

In some embodiments, the DNA origami hinge comprises two arms, wherein each of the two arms comprises a multi-layer structure.

In some embodiments, the DNA origami hinge further comprises a moiety bound to one or more staple strands. In some embodiments, the moiety comprises BHQ, FAM, BHQ2, BHQ3, AlexaFluor 488, AlexaFluor 555, AlexaFluor 647, Cy3, Cy5, quantum dots in the equivalent fluorophore wavelengths, Iowa Black RQ, Iowa Black FQ, gold nanoparticles, biotinylated oligonucleotide/Horse Radish Peroxidase (HRP)-streptavidin, or glucose oxidase-GOx. In some embodiments, the moiety comprises BHQ and/or FAM.

In some embodiments, a first arm of the DNA origami hinge comprises one or more quenchers, and wherein a second arm of the DNA origami hinge comprises one or more fluorophores. In some embodiments, the first arm of the DNA origami hinge comprises at least 30 quenchers and the second arm of the DNA origami hinge comprises at least 30 fluorophores. In some embodiments, the quencher is BHQ. In some embodiments, the fluorophore is BHQ.

In some embodiments, the one or more fluorophores and the one or more quenchers are positioned on an inner surface of the DNA origami hinge when the DNA origami hinge is in a closed configuration.

In some aspects, disclosed herein is a method of detecting a virus in a subject, comprising
a) obtaining a biological sample from the subject; and
b) detecting a nucleic acid of the virus using the biosensor of any preceding aspect.

In some embodiments, the method further comprises a step of purifying a nucleic acid from the biological sample.

In some embodiments, the virus is an RNA virus. In some embodiments, the RNA virus is a coronavirus. In some embodiments, the coronavirus comprises SARS-COV-2.

In some embodiments, the RNA virus comprises influenza, HIV, hepatitis C virus, Ebola virus, rabies virus, or Dengue virus.

In some aspects, disclosed herein is a biosensor comprising:
a DNA origami hinge that comprises one or more scaffold strands, one or more staple strands, and one or more overhang staple strands; and
a latch strand that is capable of hybridizing to the one or more overhang staple strands and a target nucleic acid, and wherein the latch strand comprises a toehold domain,
wherein the DNA origami hinger comprises a first arm and a second arm, wherein the first arm comprises one or more quenchers, and wherein the second arm comprises one or more fluorophores.

### BRIEF DESCRIPTION OF THE DRAWINGS

The accompanying figures, which are incorporated in and constitute a part of this specification, illustrate several aspects described below.

FIG. 1 shows workflow of the method using DNA origami hinge, which eliminate the steps of isolating RNA, cDNA synthesis, and replicating DNA.

FIGS. 2A-2C show examples of the DNA origami. FIGS. 2A and 2C show designs of flat DNA origami hinge. FIG. 2C shows a DNA hinge with 35 FAM/BHQ. FIG. 2B shows a design of horse trap of DNA origami.

FIG. 3A shows diagram of Hinge with orange “toehold” available for target RNA attachment. FAMs are quenched by BHQs. FIG. 3B shows diagram of Hinge after COVID 19 binding, with FAM fluorescing. FIG. 3C shows Transmission Electron Micrograph of Hinge in closed (left) and open (right) states

FIG. 4 shows conformational change of DNA origami horse trap upon detection of target sequence. The design uses same principles as before. It has advantages including: allowing for naked eye reading of results, point-of care, can still multiplex, and keeping the price low by using it repeatedly (for work, schools, sporting events, or undeserved regions and countries).

FIG. 5 shows closing mechanism of DNA origami hinge. It uses a zipper (latching) strand (in red; complementary to target, SEQ ID NO: 303) to close the hinge arms. Structures can be purified, re-closed and ready for new target, if desired. A set of 1 pair (2 overhangs) up to 6 pairs (12 overhangs) to facilitate latching-strand mediated Hinge closing. Attached to OH’s (aka handles). The sequences in FIG. 5 include SEQ ID NO: 303, SEQ ID NOs: 2-7, and SEQ ID NOs: 8-13.

FIG. 6 shows signal differences between the use of different fluorophores in DNA origami hinge. The signal different between FQ1, 2, 4, and 6 was compared. The structures were all fully open and there was a linear change between #. Typically, FAM was used, Alexa488 was included for comparisons. The 2 conferred better result than 4 FAM. Notably, the design can fit up to 51 pairs of fluorophores and quenchers.

FIGS. 7A-7C show energy cost. In the initial design, the energy cost to close is too high to close by 15 bases alone.

FIGS. 8A-8C show DNA origami hinge with a new design of angle control. The slight changes of design control the range of motion and ‘angle distribution’ of hinges. The new design still wants “default” to be open configuration while the hinge can be closed.

FIG. 9 shows zipper (7-7) and zippered latch. The figure shows that the additional designs render a higher energy change (adding the long 100 base “zipper” oligonucleotide). The zipper can be fine-tuned by changing the length of the binding ranges (7-12). Zipper (7-7) includes 100 base long (6 base toehold) that is complement to both the hinge closing and SARS-CoV-2. The sequences in FIG. 9 include SEQ ID NO: 303, SEQ ID NOs: 2-7, and SEQ ID NOs: 8-13.

FIG. 10 shows closed and open conformations of Hinge DNA origami biosensor. Schematic of closed hinge DNA origami biosensor (top left), transmission electron micrograph (TEM) of open state (top right), open hinge DNA origami biosensor (bottom left), TEM of closed state (bottom left).

FIG. 11 shows zipper (10-10) and zipper latch. Zipper (1 0-10) includes 100 base long (6 base toehold) that is complement to both the hinge closing and SARS-CoV-2. Longer individual segments provide “stronger” closing than 7-7. The sequences in FIG. 11 include SEQ ID NO: 303, SEQ ID NOs: 14-17, and SEQ ID NOs: 18-21.

FIG. 12 shows additional design changes. The figure shows standard example of ‘closed’ (left) and ‘opened’ (right) Hinge DO nanobiosensors using (36 6-6 closing helper strands).

FIG. 13 shows a proposed fabrication flow. Helpers were applied. 6-6 release off after PEG purification (without excess concentration in solution they cannot bind).

FIG. 14 shows conformation changes using helpers. The experiments were done with (24, 6-6) help, then gel.

FIGS. 15A-15D show functional characterization of Hinge DNA origami SARS-CoV-2 biosensor. FIG. 15A shows schematic of open and closed hinge DNA origami biosensor. FIG. 15B shows agarose gel electrophoresis showing open, closed, and target induced opening (incubated). FIG. 15C shows TEM of open and closed hinge DNA origami biosensors. FIG. 15D shows fluorometer measurements of target sequence (N1 Gene) open and closed hinge DNA origami biosensors after incubation for 30 minutes at 37° C.

FIGS. 16A-16D show other sequence designs. FIGS. 16A and 16B show zipper (7-7) (FIG. 16A) and zipper (10-10) (FIG. 16B) for spike gene (alternative). Same general design principals were applied. The zipper (7-7) allowed for longer toehold (switched to 6 in middle, with 1 base ssDNA for space). The changes in sequences in zipper (7-7) and (10-10) led to less prevalent secondary binding of sequence than first S-gene sequence. FIGS. 16C and 16D show zipper (7-7) (FIG. 16C) and zipper (10-10) (FIG. 16D) for nucleocapsid gene (N-gene). This zipper (7-7) design also allowed longer toehold (switched to 6 in middle, with 1 base ssDNA for space). The changes in sequences led to less prevalent secondary binding of sequence than first N-gene sequence. Sequences in FIG. 16A include SEQ ID NO: 303, SEQ ID NOs: 23-27, and SEQ ID NOs: 28-33. Sequences in FIG. 16B include SEQ ID NO: 303. SEQ ID NOs: 34-37, and SEQ ID NOs: 38-41. Sequences in FIG. 16C include SEQ ID NO: 66, SEQ ID NOs: 42-47, and SEQ ID NOs: 48-53. Sequences in FIG. 16D include SEQ ID NO: 303, SEQ ID NOs: 54-57, and SEQ ID NOs: 58-61.

FIGS. 17A-17B show original sequence of S-gene for full 7-7 zipper (FIG. 17A) and part 10-10-10-10 zipper (FIG. 17B). The sequences in FIG. 17A are SEQ ID NO: 303, SEQ ID NOs: 2-7, and SEQ ID NOs: 8-13. The sequences in FIG. 17B include SEQ ID NO: 303, SEQ ID NOs: 14-17, and SEQ ID NOs: 18-21.

FIGS. 18A-18B shows studies relating to stiffness. In FIG. 18A, in order to line up fluorophores/quenchers of the design disclosed herein, a level of stiffness is required. For trap version, the more “aligned” the top and bottom hinge arms, the better protected the molecules. Focusing on the shaded gray lines (left figure), the bottom line simulates a single layered structure and the top gray line is a tight 6-helix bundle. On the bottom right figure, the stiffness differences can be seen between a 6-helix bundle and a 18-helix bundle. The design shown herein is a square lattice 18-helix bundle (even more compact/stiff than the figure). FIG. 18B shows a comparison between the design of “nanorobot” and the structure designs disclosed herein.

FIGS. 19A-19E show that nanorobot has specifically been shown to have poor stability in low Mg environments and in low amounts of serum (both likely to occur with in vitro and in vivo use). No added salt was in RPMI (similar to collection media) “appeared stressed”. Intact nanostructures were not found after incubation in standard medium, indicating poor serum stability.

FIGS. 20A-20F show a previous structure designed (using a square lattice, like the designs in FIG. 18B) has shown remarkable resistance to low ion concentrations (FIG. 20E) and presence of serum (FIG. 20F). This again supports that nanorobot has specifically been shown to have poor stability in low Mg environments and in low amounts of serum.

FIG. 21 shows that other analysis of compact square-lattice structures has confirmed reduced degradation in nuclease/low ion environments.

FIG. 22 shows diagram depicting the detection of viruses.

FIG. 23 shows design parameters of hinge DNA origami viral/nucleic acid biosensor. Design details at vertex, middle, and ends (left); sensing ‘zipper’ designs, frontal view (top right); bottom arm potential positions of fluorophore molecules (bottom right). The numbers (3→33 and 5→35) are number IDs for each of the fluorophore/quencher sites (low numbers near the zipper and high numbers near the vertex). Fluorophore/quencher sets on the zipper overhangs were also included, which are not included with the numbering. Locations 1 and 2 are gone due to design changes. The 5T/7T helps align the overhangs, giving the zipper a better binding efficiency per structure (also eliminated the dimer structures). 5T/7T is 5 or 7 single stranded thymine bases before the overhang sequence. The design had offset ends. The “trap” design doesn’t need these to be as long. The “reachers” helps the fluorophore/quencher pairs reach each other when using the zipper. The helices of the reachers align perpendicular to the arm helices. This improves quenching efficiency (another solution for improving quenching efficiency is shown in FIG. 27). The short sequence “TATA/ATAT” holds the fluorophore/quencher pairs together in a closer configuration. Sequences in FIG. 23 include SEQ ID NO: 66, SEQ ID NOs: 42-47, and SEQ ID NOs: 48-53; SEQ ID NO: 303, SEQ ID NOs: 14-17, and SEQ ID NOs: 18-21; SEQ ID NO: 305, SEQ ID NOs: 309-318.

FIG. 24 shows sensing ‘Zipper’ nucleic acid sequences used to detect viral nucleic acids and RNAse P material. “USA N1 primer SARS-CoV-2” and “USA N3 primer SARS-CoV-2” refer to sequences that are near the US N1 and US N3 primer-targeting regions in the SARS-CoV-2 genome, same regions recommended by the FDA and CDC for detection of SARS-CoV-2. Thus, the “USA N1 primer SARS-CoV-2 Zipper-closing sequence” and “USA N3 primer SARS-CoV-2 Zipper-closing sequence” refer to the sequence of Zipper strands that targets the corresponding the US N1 and US N3 primer-targeting sites in the SARS-CoV-2 genome. These regions are resistant to mutations than the S-gene. The RNase P gene is the human RNase P gene that were used a control in the detection and diagnostic kit to indicate whether a suitable biological sample was taken from a patient. PCR primers are usually 15-30 bases and amplify a segment that is 150-300 bases long. The sequences close to their PCR regions were chosen by putting a portion of the sequence into a sequence analyzer tool or finding a good image in literature and a region that appears to be accessible to bind to the toehold sequence were selected and then the rest was designed from there. Sequences in FIG. 24 are SEQ ID NOs: 303-308.

FIG. 25 shows example results of Custom Matlab Code. Written to design overhang sequences (for standard Hinge DNA origami biosensor 10-10 design). Sequences in FIG. 25 include SEQ ID NO: 305, SEQ ID NOs: 309-318.

FIG. 26 shows hinge DNA origami viral/nucleic acid Biosensor ‘Trap design’ (latched hinge box) Viral Detection Zipper. Vertex Design (left and middle); frontal sensing zipper design (top right); ‘trap’ view for incorporation of colorimetric molecules (bottom right). Sequences in FIG. 23 include SEQ ID NO: 66, SEQ ID NOs: 42-47, and SEQ ID NOs: 48-53; SEQ ID NO: 303, SEQ ID NOs: 14-17, and SEQ ID NOs: 18-21; SEQ ID NO: 305, SEQ ID NOs: 309-318.

FIG. 27 shows improving fluorescence quenching mechanism of internal overhangs (Ohs) within hinge DNA origami biosensor. Horizontal View (top left) and resulting closed structure (bottom left), this design changes the conformation of the closed zipper to away from the closed arms and allowing for a tighter closure, improving quenching. This is compared to the alternative sensing Zipper design (top right) resulting in closed structure design (bottom right) with more space between arms, but better efficiency for closing. The right is the standard, but the quenching efficiency can be improved by changing the direction of the overhangs (left side) to the zipper. The zipper’s helices are directionally parallel to the structure’s helices in the left image, but perpendicular to the structure’s helices in the right version. Sequences in FIG. 27 include SEQ ID NO: 305, SEQ ID NOs: 309-318.

FIG. 28 shows hinge DNA origami biosensor sensitivity. Closed hinge DNA origami SARS-CoV-2 (n1 gene target) mixed for 30 minutes at 37° C. followed by fluorometer measurement Limit of detection = 50 pM. Current design has 4 fluorophore/quencher pairs. The design can detect as low as 50 pM (10⁻¹²) with 4 pairs. When structure is maximized to include 45 pairs of fluorophores/quenchers, the limited of detection can be round 50-200 femtomlar (10^{- 15}).

FIG. 29 shows multiplexing using the Hinge DNA origami biosensor. 3 types of Hinge DNA origami biosensors, RNAseP (Cy3, 554 nm excitation, 568 nm emission), SARS-CoV-2 N1 (Alexa488, 490 nm excitation, 525 nm emission), and FluA M1 (Cy5, 649 nm excitation, 666 nm emission) were mixed together and spiked in three different samples (for 30 min) with target sequences for RnaP (left), SARS-CoV-2 N1 (middle), and FluA M1 (right) followed by fluorometer, where evidence shows selective binding and detection. Each graph highest signal reflects introduction to its respective target.

FIG. 30 shows storage stability of hinge DNA origami biosensors. Hinge DNA origami biosensors were stored at various temperatures either Peg precipitated, dried, or wet, in the absence or presence of Tween™ 20 and 5% glycerol for 2 months followed by functional evaluation with target sequence followed by agarose gel electrophoresis. The data show that the biosensor is stable at room temperature, in buffer at high centration with Tween™20, Tween™20 and glycerol. The biosensor is also stable at -4° C., -20° C., and -80° C., in all conditions.

FIG. 31 shows background signal evaluation. Commercially purchased, chemically and heat inactivated whole SARS-CoV-2 virus was mixed with Triton™ X-100 and evaluated on a fluorometer. Fob = folding buffer. No biosensor was added in the solution. A combination of whole virus and Triton™ X-100 shows no difference between signals at biosensor peak (~520 nm).

FIG. 32 shows detection of SARS-CoV-2 viral RNA. Commercially purchased chemically and heat inactivated whole SARS-CoV-2 virus (Helix Elite™ Inactivated Standard) was mixed with Hinge DNA origami SARS-CoV-2 (N1 gene) biosensors (diluted to 1% Tween™20 and 5 nM Hinge) and incubated at 37° C. for 30 minutes. Samples measured on a fluorometer. Tween™ 20 is the detergent for RNA release. The whole SARS-CoV-2 virus was inactivated through standard thermal and chemical process (commercially purchased). Inactivated SARS-CoV-2 Whole Virus (Pellet) is comprised of cultured and inactivated Severe Acute Respiratory Syndrome Coronavirus 2 isolate USA/WA1/2020, and human A549 cells. The SARS-CoV-2 virus has been inactivated using chemical and heat treatments, and the A549 cells have been inactivated using thermal treatment. The viral particles are prepared in a buffered solution with materials of plant and animal origin, preservatives and stabilizers. The solution is lyophilized into a pellet. The product consists of five individually packaged pellets and five vials of molecular standard water.

FIG. 33 shows detection of SARS-CoV-2 viral RNA in the presence of detergent. Commercially purchased chemically and heat inactivated whole SARS-CoV-2 virus was mixed with Hinge DNA origami SARS-CoV-2 (N1 gene) biosensors with Triton™ X-100 in the presence or absence of SARS-CoV-2 virus and incubated at 37° C. for 30 minutes. Samples were measured on a fluorometer. Triton™ X-100 was the detergent for RNA release. Triton™ X-100 increases bulk signal of biosensor, but increases further with presence of virus and Triton™ X-100, indicating biosensor detecting nucleic material.

FIG. 34 shows the detection of Human RNase P (right), and detection of no influenza A Matrix RNA in the presence of detergent. Commercially purchased chemically and heat inactivated whole SARS-CoV-2 virus was mixed with Hinge DNA origami Influenza A (Matrix 1, Cy3 labelled) biosensors with Triton™ X-100 (left) and with Hinge DNA origami RNase P biosensors (Cy5 labelled) with Triton™ X-100 (right) in the presence or absence of SARS-CoV-2 virus and human A549 cells and incubated at 37° C. for 30 minutes. Samples were measured on a fluorometer, excitation of 510 nm (on left) and 610 nm (on right). Triton™ X-100 was the detergent for RNA release. As expected, left shows no signal change in the presence of SARS-CoV-2 virus and human cells, but right shows approximately a 2-fold change with 1 Cy5/Quencher pair in the same conditions.

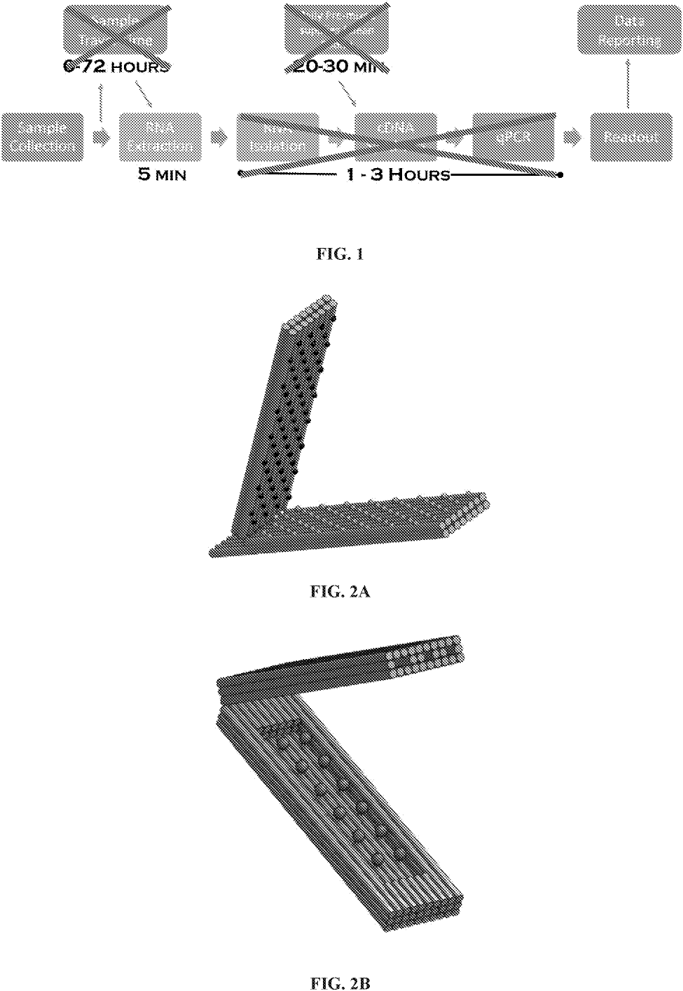

### DETAILED DESCRIPTION

Reference will now be made in detail to the embodiments of the invention, examples of which are illustrated in the drawings and the examples. This invention may, however, be embodied in many different forms and should not be construed as limited to the embodiments set forth herein.

Unless defined otherwise, all technical and scientific terms used herein have the same meaning as commonly understood to one of ordinary skill in the art to which this disclosure belongs.

### Terminology

Terms used throughout this application are to be construed with ordinary and typical meaning to those of ordinary skill in the art. However, Applicant desires that the following terms be given the particular definition as defined below.

As used herein, the article “a,” “an,” and “the” means “at least one,” unless the context in which the article is used clearly indicates otherwise.

The terms “about” and “approximately” are defined as being “‘close to” as understood by one of ordinary skill in the art. In one non-limiting embodiment, the terms are defined to be within 10%. In another non-limiting embodiment, the terms are defined to be within 5%. In still another non-limiting embodiment, the terms are defined to be within 1 %.

“Activate”, “activating”, and “activation” mean to increase an activity, response, condition, or other biological parameter. This may also include, for example, a 10% increase in the activity, response, or condition, as compared to the native or control level. Thus, the increase can be a 10, 20, 30, 40, 50, 60, 70, 80, 90, 100%, or any amount of reduction in between as compared to native or control levels.

“Administration” to a subject includes any route of introducing or delivering to a subject an agent. Administration can be carried out by any suitable route, including oral, topical, intravenous, subcutaneous, transcutaneous, transdermal, intramuscular, intra-joint, parenteral, intra-arteriole, intradermal, intraventricular, intracranial, intraperitoneal, intralesional, intranasal, rectal, vaginal, by inhalation, via an implanted reservoir, or via a transdermal patch, and the like. Administration includes self-administration and the administration by another.

The term “biocompatible” generally refers to a material and any metabolites or degradation products thereof that are generally non-toxic to the recipient and do not cause significant adverse effects to the subject.

The term “biological sample” as used herein means a sample of biological tissue or fluid. Such samples include, but are not limited to, tissue isolated from animals. Biological samples can also include sections of tissues such as biopsy and autopsy samples, frozen sections taken for histologic purposes, saliva, nasal swab, blood, plasma, serum, sputum, stool, tears, mucus, hair, and skin. Biological samples also include explants and primary and/or transformed cell cultures derived from patient tissues. A biological sample can be provided by removing a sample of cells from an animal, but can also be accomplished by using previously isolated cells (e.g., isolated by another person, at another time, and/or for another purpose), or by performing the methods as disclosed herein in vivo. Archival tissues, such as those having treatment or outcome history can also be used.

The term “biosensor” is defined as an analytical tool comprised of biological components that are used to detect the presence of target(s) and to generate a signal.

As used herein, the term “comprising” is intended to mean that the compositions and methods include the recited elements, but not excluding others. “Consisting essentially of” when used to define compositions and methods, shall mean excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions of this invention. Embodiments defined by each of these transition terms are within the scope of this invention.

A “composition” is intended to include a combination of active agent and another compound or composition, inert (for example, a detectable agent or label) or active, such as an adjuvant.

“Complementary” or “substantially complementary” refers to the hybridization or base pairing or the formation of a duplex between nucleotides or nucleic acids, such as, for instance, between the two strands of a double stranded DNA molecule or between an oligonucleotide primer and a primer binding site on a single stranded nucleic acid. Complementary nucleotides are, generally, A and T/U, or C and G. Two single-stranded RNA or DNA molecules are said to be substantially complementary when the nucleotides of one strand, optimally aligned and compared and with appropriate nucleotide insertions or deletions, pair with at least about 80% of the nucleotides of the other strand, usually at least about 90% to 95%, and more preferably from about 98 to 100%. Alternatively, substantial complementarity exists when an RNA or DNA strand will hybridize under selective hybridization conditions to its complement. Typically, selective hybridization will occur when there is at least about 65% complementary over a stretch of at least 14 to 25 nucleotides, at least about 75%, or at least about 90% complementary. See Kanehisa (1984) Nucl. Acids Res. 12:203

A “control” is an alternative subject or sample used in an experiment for comparison purposes. A control can be “positive” or “negative.”

“Decrease” can refer to any change that results in a lower level of gene expression, protein expression, amount of a symptom, disease, composition, condition, or activity. A substance is also understood to decrease the level of the gene, the protein, the composition, or the amount of the condition when the level of the gene, the protein, the composition, or the amount of the condition is less/lower relative to the output of the level of the gene, the protein, the composition, or the amount of the condition without the substance. A decrease can be any individual, median, or average decrease in a condition, symptom, activity, composition in a statistically significant amount. Thus, the decrease can be a 1, 2, 3, 4, 5, 6, 7, 8, 9, 10, 15, 20, 25, 30, 35, 40, 45, 50, 55, 60, 65, 70, 75, 80, 85, 90, 95, or 100% decrease so long as the decrease is statistically significant.

“Increase” can refer to any change that results in a higher level of gene expression, protein expression, amount of a symptom, disease, composition, condition, or activity. A substance is also understood to increase the level of the gene, the protein, the composition, or the amount of the condition when the level of the gene, the protein, the composition, or the amount of the condition is more/higher relative to the output of the level of the gene, the protein, the composition, or the amount of the condition without the substance. Also, for example, an increase can be a change in the symptoms of a disorder such that the symptoms are less than previously observed. An increase can be any individual, median, or average increase in a condition, symptom, activity, composition in a statistically significant amount. Thus, the increase can be a 1, 2, 3, 4, 5, 6, 7, 8, 9, 10, 15, 20, 25, 30, 35, 40, 45, 50, 55, 60, 65, 70, 75, 80, 85, 90, 95, or 100% increase so long as the increase is statistically significant.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom, Thus, a gene encodes a protein if transcription and translation of mRNA.

The term “nucleic acid” as used herein means a polymer composed of nucleotides, e.g. deoxyribonucleotides or ribonucleotides.

The terms “ribonucleic acid” and “RNA” as used herein mean a polymer composed of ribonucleotides.

The terms “deoxyribonucleic acid” and “DNA” as used herein mean a polymer composed of deoxyribonucleotides.

The term “oligonucleotide” denotes single- or double-stranded nucleotide multimers. Suitable oligonucleotides may be prepared by the phosphoramidite method described by Beaucage and Carruthers, Tetrahedron Lett., 22: 1859-1862 (1981), or by the triester method according to Matteucci, et al., J. Am. Chem. Soc., 103:3185 (1981), both incorporated herein by reference, or by other chemical methods using either a commercial automated oligonucleotide synthesizer or VLSIPSTM technology. When oligonucleotides are referred to as “double-stranded,” it is understood by those of skill in the art that a pair of oligonucleotides exist in a hydrogen-bonded, helical array typically associated with, for example, DNA. In addition to the 100% complementary form of double-stranded oligonucleotides, the term “double-stranded,” as used herein is also meant to refer to those forms which include such structural features as bulges and loops, described more fully in such biochemistry texts as Stryer, Biochemistry, Third Ed., (1988), incorporated herein by reference for all purposes.

The term “polynucleotide” refers to a single or double stranded polymer composed of nucleotide monomers.

The term “promoter” or “regulatory element” refers to a region or sequence determinants located upstream or downstream from the start of transcription and which are involved in recognition and binding of RNA polymerase and other proteins to initiate transcription. Promoters need not be of bacterial origin, for example, promoters derived from viruses or from other organisms can be used in the compositions, systems, or methods described herein.

The term “recombinant” refers to a human manipulated nucleic acid (e.g. polynucleotide) or a copy or complement of a human manipulated nucleic acid (e.g. polynucleotide), or if in reference to a protein (i.e, a “recombinant protein”), a protein encoded by a recombinant nucleic acid (e.g. polynucleotide). In some embodiments, a recombinant expression cassette comprising a promoter operably linked to a second nucleic acid (e.g. polynucleotide) may include a promoter that is heterologous to the second nucleic acid (e.g. polynucleotide) as the result of human manipulation (e.g., by methods described in Sambrook et al., Molecular Cloning-A Laboratory Manual, Cold Spring Harbor Laboratory, Cold Spring Harbor, N.Y., (1989) or Current Protocols in Molecular Biology Volumes 1-3, John Wiley & Sons, Inc. (1994-1998)). In another example, a recombinant expression cassette may comprise nucleic acids (e.g. polynucleotides) combined in such a way that the nucleic acids (e.g. polynucleotides) are extremely unlikely to be found in nature. For instance, human manipulated restriction sites or plasmid vector sequences may flank or separate the promoter from the second nucleic acid (e.g. polynucleotide). One of skill will recognize that nucleic acids (e.g. polynucleotides) can be manipulated in many ways and are not limited to the examples above.

The term “expression cassette” or “vector” refers to a nucleic acid construct, which when introduced into a host cell, results in transcription and/or translation of a RNA or polypeptide, respectively. In some embodiments, an expression cassette comprising a promoter operably linked to a second nucleic acid (e.g. polynucleotide) may include a promoter that is heterologous to the second nucleic acid (e.g. polynucleotide) as the result of human manipulation (e.g., by methods described in Sambrook et al., Molecular Cloning-A Laboratory Manual, Cold Spring Harbor Laboratory, Cold Spring Harbor, N.Y., (1989) or Current Protocols in Molecular Biology Volumes 1-3, John Wiley & Sons, Inc. (1994-1998)).

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%,94%, 95%, 96%, 97%, 98%, 99% or higher identity over a specified region when compared and aligned for maximum correspondence over a comparison window or designated region) as measured using a BLAST or BLAST 2.0 sequence comparison algorithms with default parameters described below, or by manual alignment and visual inspection (see, e.g., NCBI web site or the like). Such sequences are then said to be “substantially identical.” This definition also refers to, or may be applied to, the compliment of a test sequence. The definition also includes sequences that have deletions and/or additions, as well as those that have substitutions. As described below, the preferred algorithms can account for gaps and the like. Preferably, identity exists over a region that is at least about 10 amino acids or 20 nucleotides in length, or more preferably over a region that is 10-50 amino acids or 20-50 nucleotides in length. As used herein, percent (%) nucleotide sequence identity is defined as the percentage of amino acids in a candidate sequence that are identical to the nucleotides in a reference sequence, after aligning the sequences and introducing gaps, if necessary, to achieve the maximum percent sequence identity. Alignment for purposes of determining percent sequence identity can be achieved in various ways that are within the skill in the art, for instance, using publicly available computer software such as BLAST, BLAST-2, ALIGN, ALIGN-2 or Megalign (DNASTAR) software. Appropriate parameters for measuring alignment, including any algorithms needed to achieve maximal alignment over the full-length of the sequences being compared can be determined by known methods.

For sequence comparisons, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters.

One example of an algorithm that is suitable for determining percent sequence identity and sequence similarity are the BLAST and BLAST 2.0 algorithms, which are described in Altschul et al. (1977) Nuc. Acids Res. 25:3389-3402, and Altschul et al. (1990) J. Mol. Biol. 215:403-410, respectively. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al. (1990) J. Mol. Biol. 215:403-410). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) or 10, M=5, N=-4 and a comparison of both strands. For amino acid sequences, the BLASTP program uses as defaults a wordlength of 3, and expectation (E) of 10, and the BLOSUM62 scoring matrix (see Henikoff and Henikoff (1989) Proc. Natl. Acad. Sci. USA 89:10915) alignments (B) of 50, expectation (E) of 10, M=5, N=-4, and a comparison of both strands.

The BLAST algorithm also performs a statistical analysis of the similarity between two sequences (see, e.g., Karlin and Altschul (1993) Proc. Natl. Acad. Sci. USA 90:5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01.

Nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are near each other, and, in the case of a secretory leader, contiguous and in reading phase. However, operably linked nucleic acids (e.g. enhancers and coding sequences) do not have to be contiguous. Linking is accomplished by ligation at convenient restriction sites. If such sites do not exist, the synthetic oligonucleotide adaptors or linkers are used in accordance with conventional practice. In some embodiments, a promoter is operably linked with a coding sequence when it is capable of affecting (e.g. modulating relative to the absence of the promoter) the expression of a protein from that coding sequence (i.e., the coding sequence is under the transcriptional control of the promoter).

The term “subject” refers to, for example, a human in need of treatment for any purpose, and more preferably a human in need of treatment to treat a disease or disorder. The term “subject” can also refer to non-human animals, such as dogs, cats, horses, cows, pigs, sheep and non-human primates, among others.

The term “purification” as used herein refers to purification from a biological sample, i.e., blood, plasma, tissues, exosomes, or cells. As used herein the term “isolated” or “purified” when used in the context of, e.g., a nucleic acid, refers to a nucleic acid of interest that is at least 60% free, at least 75% free, at least 90% free, at least 95% free, at least 98% free, and even at least 99% free from other components with which the nucleic acid is associated with prior to purification.

### Biosensors Using a DNA Origami Hinge

The current methods used for detecting a virus are mainly RT-PCR-based assays. Such methods are time-consuming and labor intensive. The methods require an RNA isolation step that can take 45-60 minutes. Further, the purification of the RNA can affect the accuracy of the RT-PCR testing results. Therefore, a new system and method for accurate and fast detection of an infection or a disorder is needed. The biosensors disclosed herein allow for rapid and inexpensive detection of target nucleic acid sequences. Importantly, the biosensors generate a greater signal per copy upon detection of a target. Further, in some embodiments, no RNA purification step is required.

In some aspects, disclosed herein is a DNA-based biosensor constructed via the DNA origami molecular self-assembly process. In some embodiments, the biosensor is based on a hinge-like design consisting of two or more arms that are initially held in a closed configuration by a latching interaction. In some embodiments, the hinge-like design is shown, for example, in FIG. 2A, FIG. 2B, or FIG. 2C.

Accordingly, in some aspects, disclosed herein is a biosensor comprising:
a DNA origami hinge that comprises one or more scaffold strands, one or more staple strands, and one or more overhang staple strands; and
a latch strand that is capable of hybridizing to the one or more overhang staple strands and a target nucleic acid, and wherein the latch strand comprises a toehold domain.

DNA origami structures incorporate DNA as a building material to make nanoscale shapes. In general, the DNA origami process involves the folding of one or more long “scaffold” strands into a particular shape using a plurality of rationally designed “staple” strands. The sequences of the staple strands are designed such that they hybridize to particular portions of the scaffold strands and, in doing so, force the scaffold strands into a particular shape. Methods useful in the making of DNA origami structures can be found, for example, in Rothemund, P. W., Nature 440:297-302 (2006); Douglas et al., Nature 459:414-418 (2009); Dietz et al., Science 325:725-730 (2009); and U.S. Pat. App. Pub. Nos. 2007/0117109, 2008/0287668, 2010/0069621 and 2010/0216978, each of which are incorporated by reference in their entireties.

In some embodiments, the one or more scaffold strands comprise a polynucleotide sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 1.

In some embodiments, the one or more staple strands comprise a polynucleotide sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NOs: 68-299. In some embodiments, the one or more staple strands comprise a polynucleotide sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NOs: 68-124. In some embodiments, the one or more staple strands comprise a polynucleotide sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NOs: 134-184.

In some embodiments, the one or more overhang staple strands comprise a polynucleotide sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NOs: 187-201, 203-218, or 221-229.

In some embodiments, the DNA origami hinge disclosed herein comprises a first arm and a second arm. In some embodiments, the first arm comprises one or more staple strands selected from the group consisting of SEQ ID NOs: 134-184. In some embodiments, the second arm comprises one or more staple strands selected from the group consisting of SEQ ID NOs: 68-124.

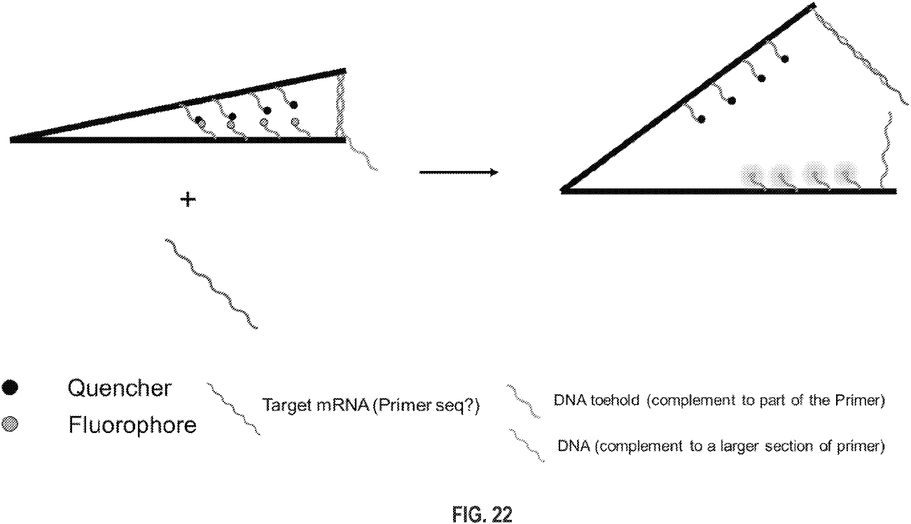

It should be understood and herein contemplated that the DNA origami hinge disclosed herein confers a greater stability in comparison to prior DNA origami designs, such as DNA nanorobot (or DNA robot). A DNA nanorobot has a single layer structure, causing the nanorobot to have a poor stability in low magnesium environments and in low amounts of serum, which both can occur with in vitro and in vivo applications. Unlike the prior designs, in some embodiments, the DNA origami hinge disclosed herein comprises one or more bundles that are multi-layers of helices (for example, FIG. 22, right panel). This structure shows improved stability even in low magnesium environments, serum, nasopharyngeal fluid, or saliva. Accordingly, in some embodiments, the DNA origami hinge comprises two arms, wherein each of the two arms comprises a multi-layer structure.

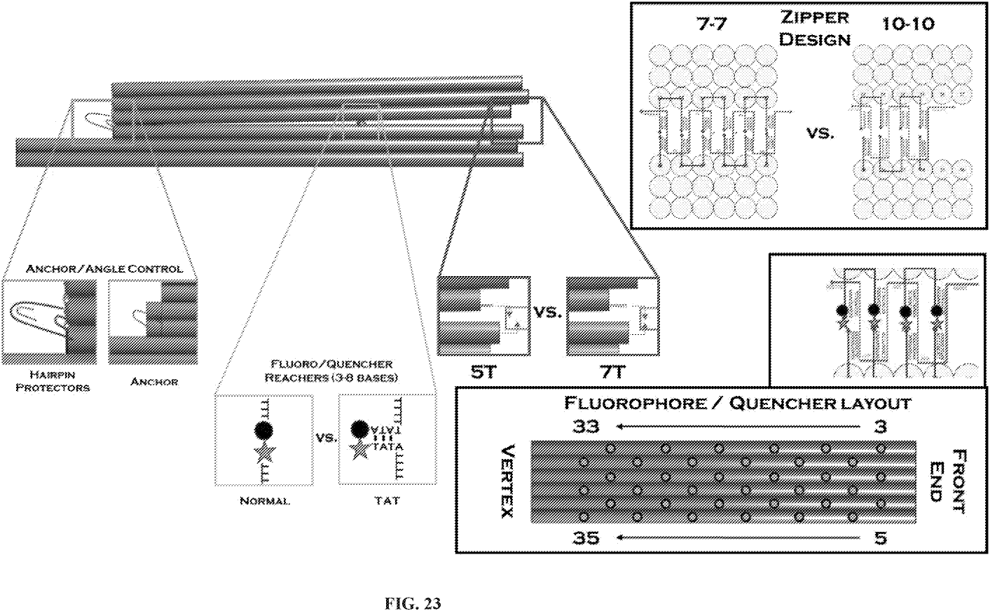

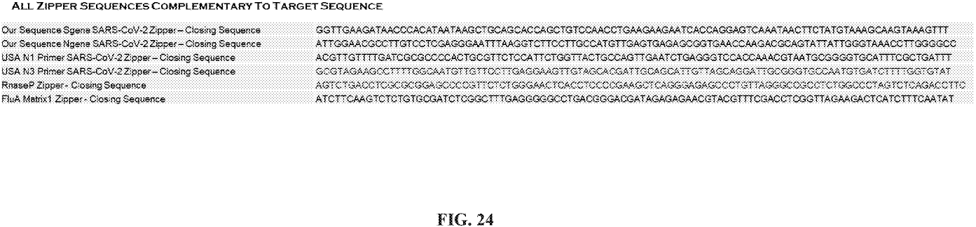

In some embodiments, the first and the second arms are connected by the scaffold strands at the vertex of the hinge. In some embodiments, the first and the second arms are further connected by one or more hinge connectors. “Hinge connector” used herein refers to staple strands that hybridize with the scaffold DNA loops at the vertex of the hinge. The length and composition of the hinge connector sequences help the “angle control” of the hinge (e.g., control an angle of an open hinge) and may or may not be used depending on the hinge version and desired angle distribution (e.g., an angle of at least about 5 degrees, at least about 10 degrees, at least about 20 degrees, at least about 30 degrees, at least about 40 degrees, at least about 50 degrees, at least about 60 degrees, at least about 70 degrees, at least about 80 degrees, at least about 90 degrees, at least about 100 degrees, at least about 120 degrees, at least about 140 degrees, at least about 160 degrees, or at least about 180 degrees). In some embodiments, the hinge-like design is shown, for example, in FIGS. 8A-8C and FIG. 23.

In some embodiments, the hinge connector comprises a polynucleotide sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NOs: 125-130.

In general, an inner surface is any surface area of the DNA origami hinge that is precluded from interacting a particle (e.g., a particle bigger than about 1 nm, 2 nm, 3 nm, 4 nm, 5 nm, 6 nm, 7 nm, 8 nm, 9 nm, 10 nm, 15 nm, 20 nm, 15 nm, 30 nm, 35 nm, 40 nm, 45 nm, 50 nm, 55 nm, 60 nm, 70 nm, 80 nm, 90 nm, 100 nm, 150 nm, 200 nm, 250 nm, 300 nm, 350 nm, 400 nm, 450 nm, 500 nm, 600 nm, 700 nm, 800 nm, 900 nm, 1000 nm, 5 µm, or 10 µm) outside the DNA origami hinge, while an outer surface is any surface area of the DNA origami hinge that is not precluded from interacting with a particle (e.g., a particle bigger than about 1 nm, 2 nm, 3 nm, 4 nm, 5 nm, 6 nm, 7 nm, 8 nm, 9 nm, 10 nm, 15 nm, 20 nm, 15 nm, 30 nm, 35 nm, 40 nm, 45 nm, 50 nm, 55 nm, 60 nm, 70 nm, 80 nm, 90 nm, 100 nm, 150 nm, 200 nm, 250 nm, 300 nm, 350 nm, 400 nm, 450 nm, 500 nm, 600 nm, 700 nm, 800 nm, 900 nm, 1000 nm, 5 µm, or 10 µm) outside the DNA origami hinge.

The latch strand (also referred to as the “zipper” or “zipper strand”) used herein refers to a single stranded nucleic acid sequence that serves to close the first arm and the second arm of the DNA origami hinge, sense a target sequence (e.g., a nucleic acid sequence), and respond to the target sequence via a complementary base pair binding to cause the DNA origami hinge to change from a closed to an open configuration. It should be understood herein that the latch strand is not a part of the DNA origami hinge. Therefore, in some examples, the design disclosed herein overcomes the challenges of reusing the DNA origami hinges by, for example, reloading the latch strands onto the DNA origami hinges.

In some examples, the overhang staple strand described herein comprises one or more fastening sequences located near the 5′-end and/or 3′-end of the overhang staple strand. It is also contemplated herein that the latch strand keeps the DNA origami hinge in a closed configuration by base pairing with one or more fastening sequences of one or more overhang staple strands, wherein the one or more fastening sequences protrude from DNA helices from a first arm and DNA helices from a second arm of the DNA origami hinge. The 5′-end portion and/or 3′-end portion of the latch strand includes free unbound sequences known as the “toehold domains”. The toehold domains facilitate a toehold-mediated strand displacement by the target sequence to release the latch strand from the first and second arms of the DNA origami hinge. This results in the DNA origami hinge to change from a closed to open configuration.

In some examples, the fastening sequences of the first hinge arm hybridize to the fastening sequences of the second hinge arm to facilitate hinge closing.

In some embodiments, the latch strand comprises at least about 20 nucleotides, 22, nucleotides, 24 nucleotides, 26 nucleotides, 28 nucleotides, 30 nucleotides, 32 nucleotides, 34 nucleotides, 36 nucleotides, 38 nucleotides, 40 nucleotides, 42 nucleotides, 44 nucleotides, 46 nucleotides, 48 nucleotides, 50 nucleotides, 52 nucleotides, 54 nucleotides, 56 nucleotides, 58 nucleotides, 60 nucleotides, 62 nucleotides, 64 nucleotides, 66 nucleotides, 68 nucleotides, 70 nucleotides, 72 nucleotides, 74 nucleotides, 76 nucleotides, 78 nucleotides, 80 nucleotides, 82 nucleotides, 84 nucleotides, 86 nucleotides, 88 nucleotides, 90 nucleotides, 92 nucleotides, 94 nucleotides, 96 nucleotides, 98 nucleotides, 100 nucleotides, 110 nucleotides, 120 nucleotides, 130 nucleotides, 140 nucleotides, 150 nucleotides, 160 nucleotides, 170 nucleotides, 180 nucleotides, 190 nucleotides, 200 nucleotides, 250 nucleotides, 300 nucleotides, 350 nucleotides, 400 nucleotides, 450 nucleotides, or 500 nucleotides. In some embodiments, the latch strand is an RNA or a DNA. In some embodiments, the latch strand comprises about 100 nucleotides. In some embodiments, the latch strand comprises a polynucleotide sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 62, 64, 66, 303, 304, 305, 306, 307, or 308.

In some embodiments, the latch strand comprises at least 1 nucleotide, 2 nucleotides, 3 nucleotides, 4 nucleotides, 5 nucleotides, 6 nucleotides, 7 nucleotides, 8 nucleotides, 9 nucleotides, or 10 nucleotides complementary to each of the one or more overhang staple strands. In some embodiments, the toehold domain does not hybridize the fastening sequence. In some embodiments, the latch strand has a higher binding affinity to the target nucleic acid than to the one or more fastening sequences. In some embodiments, the latch strand has at least 85% (at least 90%, at least 95%, at least 96%, at least 97%, at least 98%, or at least 99%) reverse complementarity to the target sequence or a fragment thereof. In some embodiments, the latch strand has 100% reverse complementarity to the target sequence or a fragment thereof.

In some embodiments, the latch strand comprises one or more toehold domains. In some embodiments, the toehold domain comprises at least about 2 nucleotides, 3 nucleotides, 4 nucleotides, 5 nucleotides, 6 nucleotides, 7 nucleotides, 8 nucleotides, 9 nucleotides, 10 nucleotides, 11 nucleotides, 12 nucleotides, 13 nucleotides, 14 nucleotides, 15 nucleotides, 16 nucleotides, 17 nucleotides, 18 nucleotides, 19 nucleotides, 20 nucleotides, 25 nucleotides, 30 nucleotides, 35 nucleotides, 40 nucleotides, 45 nucleotides, 50 nucleotides, 55 nucleotides, 60 nucleotides, 65 nucleotides, 70 nucleotides, 75 nucleotides, 80 nucleotides, 85 nucleotides, 90 nucleotides, 95 nucleotides, or 100 nucleotides. In general, the toehold domain used herein can be a polynucleotide sequence of any length and is complementary to a target sequence (e.g., a viral nucleic acid or a tumor-derived/tumor-specific nucleic acid). In some embodiments, the toehold domain comprises a polynucleotide sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 63, 65, 67, 193, or 318.

In some embodiments, the toehold domain has at least 85% (at least 90%, at least 95%, at least 96%, at least 97%, at least 98%, or at least 99%) reverse complementarity to the target sequence or a fragment thereof. In some embodiments, the toehold domain has 100% reverse complementarity to the target sequence or a fragment thereof.

Overhang staple strands comprise fastening sequences (herein also termed “overhang sequences” or “OH”) that protrude from the front edge of each of the first and/or the second hinge arms that hybridize to the latch/zipper strand to facilitate hinge closing. In some examples, the fastening sequences locate at the 5′-end and the 3′-end of the overhang staple strand. In some embodiments, the overhang staple strand comprises a polynucleotide sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NOs: 205-211. In some embodiments, the overhang staple strand comprises a polynucleotide sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NOs: 187-201, 203-218, or 221-229.

In some embodiments, the fastening sequence comprises a polynucleotide sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NOs: 2-13. In some embodiments, the fastening sequence comprises a polynucleotide sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NOs: 14-21. In some embodiments, the fastening sequence comprises a polynucleotide sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NOs: 22-33. In some embodiments, the fastening sequence comprises a polynucleotide sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NOs: 34-41. In some embodiments, the fastening sequence comprises a polynucleotide sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NOs: 42-53. In some embodiments, the fastening sequence comprises a polynucleotide sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NOs: 54-61. In some embodiments, the fastening sequence comprises a polynucleotide sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NOs: 310-317.

In some embodiments, the fastening sequence comprises one or more thymine bases (including, for example, at least one thymine base, at least two thymine bases, at least three thymine bases, at least four thymine bases, at least five thymine bases, at least six thymine bases, at least seven thymine bases, at least eight thymine bases, at least nine thymine bases, or at least ten thymine bases) at the 5′-end and/or 3′-end of the fastening sequence. In some embodiments, the fastening sequence comprises five thymine bases at its 5′-end and/or 3′-end. In some embodiments, the fastening sequence comprises seven thymine bases at its 5′-end and/or 3′-end. In some embodiments, the design of the thymine bases is shown, for example, in FIG. 23, which shows that poly thymine bases are before the fastening sequence to allow the fastening sequence on one arm reach the fastening sequence on the other arm.

In some embodiments, the target nucleic acid displaces the one or more fastening sequences when hybridizing to the latch strand. In some embodiments, the DNA origami hinge is in an open configuration when the latch strand is not hybridized to the fastening sequences.

In some embodiments, the target nucleic acid is a single stranded nucleic acid. In some embodiments, the target nucleic acid is a tumor-specific nucleic acid. In some embodiments, the target nucleic acid is a nucleic acid derived from a pathogen (for example, a virus, a bacterium, a fungus, or a parasite). In some embodiments, the target nucleic acid is a viral RNA or a viral DNA. In some embodiments, target nucleic acid is an RNA or a DNA of a coronavirus.

Coronaviruses constitute the subfamily Orthocoronavirinae, in the family Coronaviridae, order Nidovirales, and realm Riboviria. They are enveloped viruses with a positive-sense single-stranded RNA genome and a nucleocapsid of helical symmetry. The genome size of coronaviruses ranges from approximately 27 to 34 kilobases. The structure of coronavirus generally consists of the following: spike protein (S), hemagglutinin-esterase dimer (HE), a membrane glycoprotein (M), an envelope protein (E) a nucleocapsid protein (N) and RNA. The coronavirus family comprises genera including, for example, alphacoronavius (e.g., Human coronavirus 229E, Human coronavirus NL63, Miniopterus bat coronavirus 1, Miniopterus bat coronavirus HKU8, Porcine epidemic diarrhea virus, Rhinolophus bat coronavirus HKU2, Scotophilus bat coronavirus 512), betacoronavirus (e.g., SARS-CoV-2, Betacoronavirus 1, Human coronavirus HKU1, Murine coronavirus, Pipistrellus bat coronavirus HKU5, Rousettus bat coronavirus HKU9, Severe acute respiratory syndrome-related coronavirus, Tylonycteris bat coronavirus HKU4, Middle East respiratory syndrome-related coronavirus (MERS), Human coronavirus OC43, Hedgehog coronavirus 1 (EriCoV)), gammacoronavirus (e.g., Beluga whale coronavirus SW1, Infectious bronchitis virus), and deltacoronavirus (e.g., Bulbul coronavirus HKU11, Porcine coronavirus HKU15). In some embodiments, the target nucleic acid is a SARS-COV-2 RNA or a SARS-COV-2 DNA (e.g., an RNA/ DNA encoding a spike protein (S), hemagglutinin-esterase dimer (HE), a membrane glycoprotein (M), an envelope protein (E), or a nucleocapsid protein (N) of a SARS-COV-2) or a fragment thereof. The target nucleic acid can be a viral RNA or viral DNA of any SARS-CoV-2 variant, including, for example, an Alpha variant (B.1.1.7, Q.1-Q.8), a Beta variant (B.1.351, B.1.351.2, B.1.351.3), a Delta variant (B.1.617.2 and AY.1 sublineages), a Gamma variant (P.1, P.1.1, P.1.2), an Epsilon variant (B.1.427 and B.1.429), an Eta variant (B.1.525), an Iota variant (B.1.526), a Kappa variant (B.1.617.1), B.1.617.3, a Mu variant (B.1.621, B.1.621.1), or a Zeta variant (P.2).

In some embodiments, the target nucleic acid is a nucleic acid of a virus, wherein the virus comprises Herpes Simplex virus- 1, Herpes Simplex virus-2, Varicella-Zoster virus, Epstein-Barr virus, Cytomegalovirus, Human Herpes virus-6, Variola virus, Vesicular stomatitis virus, Hepatitis A virus, Hepatitis B virus, Hepatitis C virus, Hepatitis D virus, Hepatitis E virus, Rhinovirus, Influenza virus A, Influenza virus B, Measles virus, Polyomavirus, Human Papillomavirus, Respiratory syncytial virus, Adenovirus, Coxsackie virus, Dengue virus, Mumps virus, Poliovirus, Rabies virus, Rous sarcoma virus, Reovirus, Yellow fever virus, Zika virus, Ebola virus, Marburg virus, Lassa fever virus, Eastern Equine Encephalitis virus, Japanese Encephalitis virus, St. Louis Encephalitis virus, Murray Valley fever virus, West Nile virus, Rift Valley fever virus, Rotavirus A, Rotavirus B, Rotavirus C, Sindbis virus, Simian Immunodeficiency virus, Human T-cell Leukemia virus type-1, Hantavirus, Rubella virus, Simian Immunodeficiency virus, Human Immunodeficiency virus type-1, or Human Immunodeficiency virus type-2. In some embodiments, the target nucleic acid is an RNA or a DNA or an influenza virus (including, for example, influenza A virus or influenza B virus).

In some embodiments, the target nucleic acid comprises a SARS-COV-2 S gene or a fragment thereof, wherein the target nucleic acid comprises a polynucleotide sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 300.

In some embodiments, the target nucleic acid comprises a SARS-COV-2 N gene or a fragment thereof, wherein the target nucleic acid comprises a polynucleotide sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 301.

In some embodiments, the target sequences are the sequences near the USA N1 and USA N3 primer-targeting regions in the SARS-CoV-2 genome. These regions are recommended by the FDA and CDC for detection of SARS-CoV-2. In some embodiments, the target sequences are human RNAse P gene or influenza A matrix protein encoding gene. Thus, the “USA N1 primer SARS-CoV-2 Zipper-closing sequence” and “USA N3 primer SARS-CoV-2 Zipper-closing sequence” refer to the sequences of Zipper strands that target the corresponding the US N1 and US N3 primer-targeting sites in the SARS-CoV-2 genome. These regions are more resistant to mutations than SARS-CoV-2 S gene. In some embodiments, the target nucleic acid comprises a polynucleotide sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 309. Accordingly, in some embodiments, the latch strand comprises a polynucleotide sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NO: 62, 64, 66, 303, 304, 305, 306, 307, or 308.

In some embodiments, the DNA origami hinge further comprises a moiety bound to one or more staple strands. In some examples, the moiety is located on the inner surface when the DNA origami hinge is in a close configuration. The moiety can be bound to the one or more staple strands using any method known in the art. For example, the moiety can be covalently bonded to the one or more staple strands or by hybridizing to the free unbound sequence of the one or more staple strands. The moiety can also be indirectly attached to the one or more staple strands through, for example, another nucleic acid sequence or a linker.

Any type of moiety can be bound to the staple strands of the DNA origami hinge. In some examples, the moiety used herein can produce colorimetric, fluorescence, or radiation readouts. In some embodiments, the moiety comprises a fluorophore and/or a quencher. In some embodiments, the moiety comprises BHQ, FAM, BHQ2, BHQ3, AlexaFluor 488, AlexaFluor 555, AlexaFluor 647, Cy3, Cy5, quantum dots in the equivalent fluorophore wavelengths, Iowa Black RQ, Iowa Black FQ, gold nanoparticles, biotinylated oligonucleotide/Horse Radish Peroxidase (HRP)-streptavidin and/or glucose oxidase-GOx. The fluorophore and the quencher used herein can be any of those known in the art, including, for example, FAM fluorescent molecules and Black Hole quenchers (BHQ).

Other detection methods can include electrochemical and surface plasmon resonance-based detection schemes (see for example, “An electrochemical biosensor exploiting binding-induced changes in electron transfer of electrode-attached DNA origami to detect hundred nanometer-scale targets.” Nanoscale, 2020, 12, 13907; see also “Binding to Nanopatterned Antigens is Dominated by the Spatial Tolerance of Antibodies.” Nat Nanotechnol. 2019 February; 14(2): 184-190.). The sensor can be immobilized on a surface, usually gold-coated (or some conductive coating). The presence of the sensor, made from charged DNA material, and/or binding of the target influences the surface electrical and/or optical properties, which provides a measurable readout. Alternatively, the DNA origami can be modified with a reporter molecule that interacts with the surface to change a readout signal. Because the biosensor herein is based on a large conformational change, these readout methods can provide a strong signal change.

Lining up the fluorophores with their respective quencher thereof is challenging. The DNA origami hinge design disclosed herein overcomes these challenges. Accordingly, in some examples, a first arm of the DNA origami hinge comprises one or more quenchers, and wherein a second arm of the DNA origami hinge comprises one or more fluorophores. In some embodiments, the staple strands bounded to the quenchers comprises a polynucleotide sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NOs: 230-264. In some embodiments, the staple strands bounded to the fluorophores comprises a polynucleotide sequence at least 60% (for example, at least 60%, at least 65%, at least 70%, at least 75%, at least 80%, at least 85%, at least 90%, at least 95%, at least 96%, at least 97%, at least 98%, or at least 99%) identical to SEQ ID NOs: 265-299.

In some embodiments, the staple strand bounded to the quencher/fluorophore comprises a short nucleotide sequence (also herein referred as “reacher” sequence) at the end of staple strand and directly/non-directly linked to the quencher/fluorophore, wherein the reacher sequence linked to the quencher is complementary to the reacher sequence linked the paired fluorophore. In some embodiments, the reacher sequence is TATA. In some embodiments, the reacher sequence is ATAT. In some embodiments, the design of the reacher sequence is shown, for example, in FIG. 23. The “reacher” helps the fluorophore/quencher pairs reach each other when using the zipper.

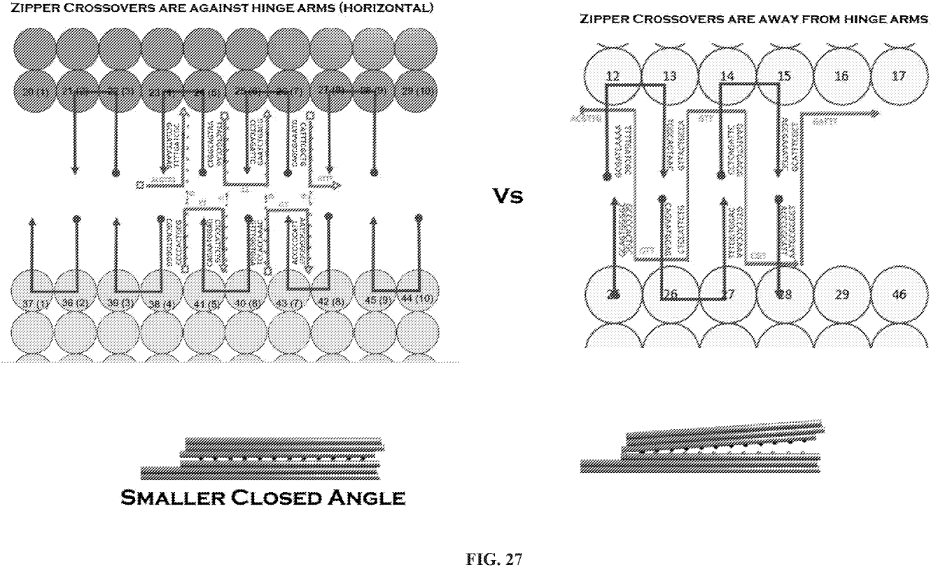

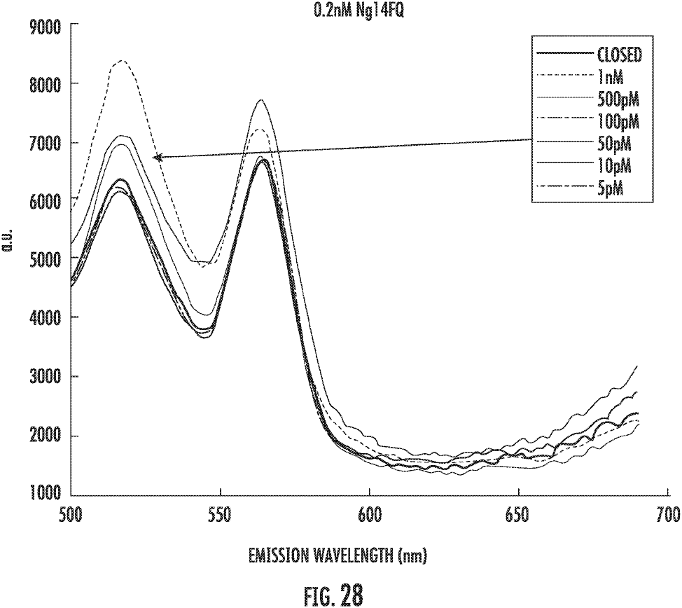

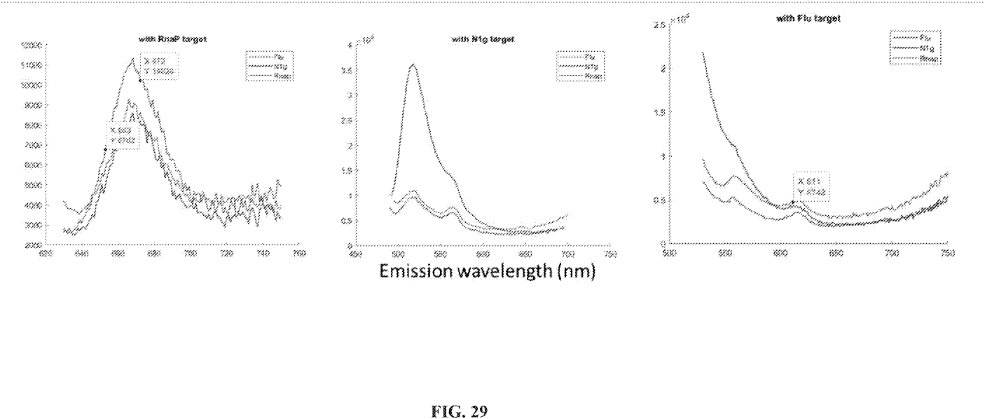

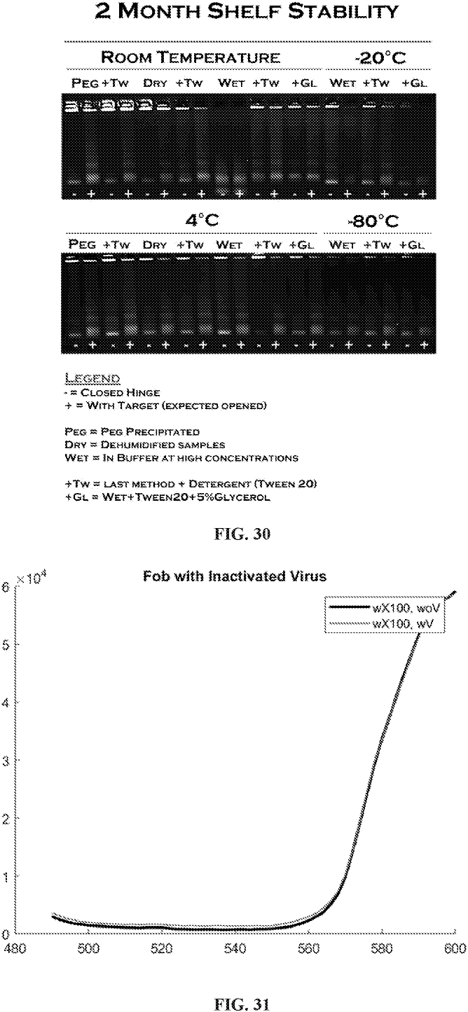

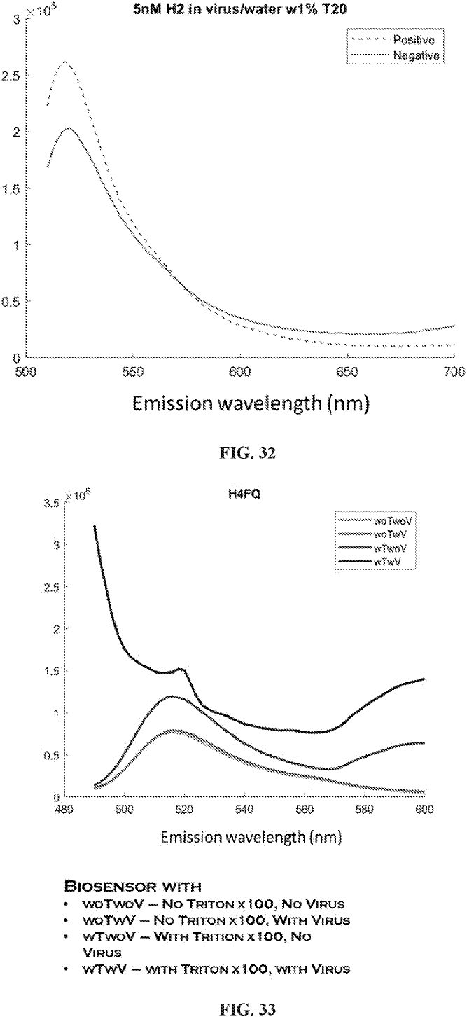

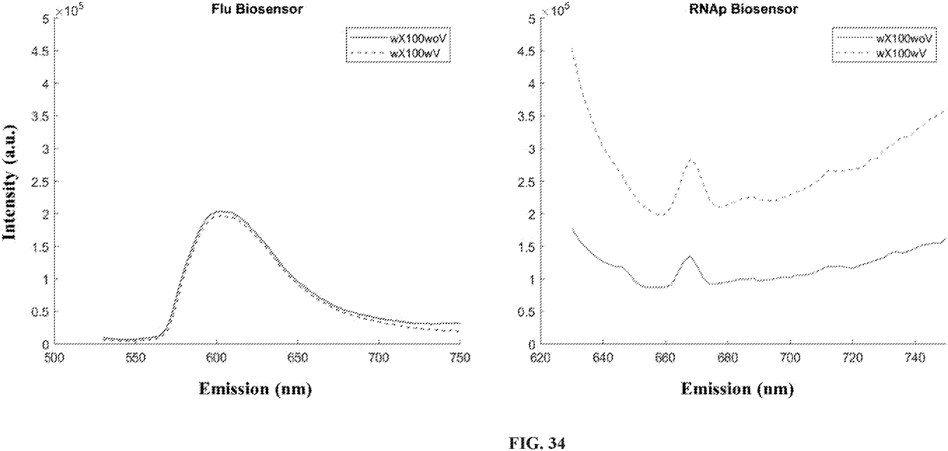

The quenching efficiency can be improved by changing the direction of the overhangs (left side) to the zipper. In some embodiments, the latch strand/zipper helices are directionally parallel to the structure’s helices (for example, as shown in FIG. 27, left side). In some embodiments, the latch strand/zipper helices are perpendicular to the structure’s helices (for example, as shown in FIG. 27, right side).

In some embodiments, the first arm of the DNA origami hinge comprises at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 quenchers. In some embodiments, the second arm of the DNA origami hinge comprises at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 fluorophores. In some embodiments, the first arm of the DNA origami hinge comprises at least 2 quenchers and the second arm of the DNA origami hinge comprises at least 2 fluorophores. In some embodiments, the first arm of the DNA origami hinge comprises at least 4 quenchers and the second arm of the DNA origami hinge comprises at least 4 fluorophores. In some embodiments, the first arm of the DNA origami hinge comprises at least 30 quenchers and the second arm of the DNA origami hinge comprises at least 30 fluorophores. In some embodiments, the first arm of the DNA origami hinge comprises at least 45 quenchers and the second arm of the DNA origami hinge comprises at least 45 fluorophores.

The limit of detection of the DNA origami hinge described herein can be less than 1×10^{- 8} molar concentration (M), less than 1 × 10⁻⁹ M, less than 1 × 10-10 M, less than 1 × 10⁻¹⁰ M, less than 1 × 10⁻¹¹ M, less than 1 × 10⁻¹² M, less than 1 × 10⁻¹³ M, less than 1 × 10-¹⁴ M, less than 1 × 10⁻¹⁵ M, less than 1 × 10⁻¹⁶ M, less than 1 × 10⁻¹⁷ M, or less than 1 × 10-¹⁸ M. The limit of detection can decrease as the number of fluorophores/quenchers increases. In some embodiments, the limit of detection of an DNA origami hinge having 4 pairs of fluorophores and quenchers is about 5 × 10⁻ 11 M. In some embodiments, the limit of detection of an DNA origami hinge having 45 pairs of fluorophores and quenchers is from about 5 × 10⁻¹⁴ M to about 2× 10⁻¹³ M.

In some embodiments, an increase in fluorescence emission is detected when the DNA origami hinge is in the open configuration as compared to the fluorescence emission detected when the DNA origami hinge is in the closed configuration.

In some aspects, disclosed herein is a biosensor comprising:
a DNA origami hinge that comprises one or more scaffold strands, one or more staple strands, and one or more overhang staple strands; and
a latch strand that is capable of hybridizing to the one or more overhang staple strands and a target nucleic acid, and wherein the latch strand comprises a toehold domain,
wherein the DNA origami hinger comprises a first arm and a second arm, wherein the first arm comprises one or more quenchers, and wherein the second arm comprises one or more fluorophores.

### Methods

In some aspects, disclosed herein is a method of detecting a nucleic acid in a subject, comprising
a) obtaining a biological sample from the subject; and
b) detecting the nucleic acid in the biological sample using the biosensor disclosed herein.

As discussed above, the biosensors disclosed herein are for accurate and fast detection of an infection or a disorder. The biosensors are highly stable in low magnesium environments or in biological samples. The biosensors also show an improvement in nuclease resistance. Accordingly, in some embodiments, the method disclosed herein does not require a step of purification of nucleic acid. In some embodiments, the method disclosed herein further comprises a step of purifying a nucleic acid from the biological sample.

In some embodiments, the biological sample is a saliva sample or a nasal swab sample. In some embodiments, the biological sample is a nasopharyngeal fluid sample.

In some embodiments, the nucleic acid is a nucleic acid of a pathogen (for example, a virus, a bacterium, a fungus, or a parasite) or a disease-specific nucleic acid (e.g., a tumor-specific nucleic acid).

In some embodiments, the nucleic acid is a nucleic acid of a virus, wherein the virus comprises Herpes Simplex virus- 1, Herpes Simplex virus-2, Varicella-Zoster virus, Epstein-Barr virus, Cytomegalovirus, Human Herpes virus-6, Variola virus, Vesicular stomatitis virus, Hepatitis A virus, Hepatitis B virus, Hepatitis C virus, Hepatitis D virus, Hepatitis E virus, Rhinovirus, Coronavirus, Influenza virus A, Influenza virus B, Measles virus, Polyomavirus, Human Papillomavirus, Respiratory syncytial virus, Adenovirus, Coxsackie virus, Dengue virus, Mumps virus, Poliovirus, Rabies virus, Rous sarcoma virus, Reovirus, Yellow fever virus, Zika virus, Ebola virus, Marburg virus, Lassa fever virus, Eastern Equine Encephalitis virus, Japanese Encephalitis virus, St. Louis Encephalitis virus, Murray Valley fever virus, West Nile virus, Rift Valley fever virus, Rotavirus A, Rotavirus B, Rotavirus C, Sindbis virus, Simian Immunodeficiency virus, Human T-cell Leukemia virus type-1, Hantavirus, Rubella virus, Simian Immunodeficiency virus, Human Immunodeficiency virus type-1, or Human Immunodeficiency virus type-2.

In some embodiments, the virus is an RNA virus. In some embodiments, the RNA virus is a coronavirus. In some embodiments, the coronavirus comprises SARS, SARS-COV-2, or MERS.

In some embodiments, the nucleic acid is a SARS-COV-2 RNA or a SARS-COV-2 DNA (e.g., an RNA/ DNA encoding a spike protein (S), hemagglutinin-esterase dimer (HE), a membrane glycoprotein (M), an envelope protein (E), or a nucleocapsid protein (N) of a SARS-COV-2) or a fragment thereof.

Accordingly, in some aspects, disclosed herein is a method of detecting SARS-COV-2 in a subject, comprising
a) obtaining a biological sample from the subject; and
b) detecting a nucleic acid of SARS-COV-2 using the biosensor disclosed herein.

In some embodiments, the RNA virus comprises an influenza virus (e.g., influenza A virus or influenza B virus), HIV, hepatitis C virus, Ebola virus, rabies virus, or Dengue virus. According, in some embodiments, disclosed herein is a method of detecting a viral nucleic acid of influenza, HIV, hepatitis C virus, Ebola virus, rabies virus, or Dengue virus.

In some embodiments, the nucleic acid is a nucleic acid of an influenza virus (e.g., an RNA encoding a PB1 protein, a PB2 protein, a PA protein, a neuraminidase protein (NA), a hemagglutinin protein (HA), a nucleocapsid protein (NP), nonstructural protein (NS), matrix protein M1 and M2 of an influenza virus) or a fragment thereof.

Accordingly, in some aspects, disclosed herein is a method of detecting an influenza virus in a subject, comprising
a) obtaining a biological sample from the subject; and
b) detecting a nucleic acid of influenza virus using the biosensor disclosed herein.

In some embodiment, the influenza virus is influenza A virus or influenza B virus.

In some embodiments, the nucleic acid is a nucleic acid of bacteria, wherein the bacteria comprises Mycobaterium tuberculosis, Mycobaterium bovis, Mycobaterium bovis strain BCG, BCG substrains, Mycobaterium avium, Mycobaterium intracellular, Mycobaterium africanum, Mycobaterium kaususii, Mycobaterium marinum, Mycobaterium ulcerans, Mycobaterium avium subspecies paratuberculosis, Nocardia asteroides, other Nocardia species, Legionella pneumophila, other Legionella species, Acetinobacter baumanii, Salmonella typhi, Salmonella enterica, other Salmonella species, Shigella boydii, Shigella dysenteriae, Shigella sonnei, Shigella flexneri, other Shigella species, Yersinia pestis, Pasteurella haemolytica, Pasteurella multocida, other Pasteurella species, Actinobacillus pleuropneumoniae, Listeria monocytogenes, Listeria ivanovii, Brucella abortus, other Brucella species, Cowdria ruminantium, Borrelia burgdorferi, Bordetella avium, Bordetella pertussis, Bordetella bronchiseptica, Bordetella trematum, Bordetella hinzii, Bordetella pteri, Bordetella parapertussis, Bordetella ansorpii, other Bordetella species, Burkholderia mallei, Burkholderia psuedomallei, Burkholderia cepacian, Chlamydia pneumoniae, Chlamydia trachomatis, Chlamydia psittaci, Coxiella burnetii, Rickettsial species, Ehrlichia species, Staphylococcus aureus, Staphylococcus epidermidis, Streptococcus pneumoniae, Streptococcus pyogenes, Streptococcus agalactiae, Escherichia coli, Vibrio cholerae, Campylobacter species, Neiserria meningitidis, Neiserria gonorrhea, Pseudomonas aeruginosa, other Pseudomonas species, Haemophilus influenzae, Haemophilus ducreyi, other Hemophilus species, Clostridium tetani, Clostridium difficile, other Clostridium species, Yersinia enterolitica, and other Yersinia species, or Mycoplasma species.

In some embodiments, the method herein is for detection of a tumor-specific nucleic acid, including, for example, tumor-specific nucleic acids or oncogenes (derived from point mutations, amplification, or fusion) that encode tumor antigens, such as a glioma-associated antigen, carcinoembryonic antigen (CEA), EGFRvIII, EGFR, FAP, B7H3, Kit, CA LX, CS-1, BCMA, β-human chorionic gonadotropin, alphafetoprotein (AFP), cyclin Bl, lectin-reactive AFP, Fos-related antigen 1, ADRB3, thyroglobulin, AKAP-4, OY-TESI, CLL-1, fucosyl GM1, GloboH, MN-CA IX, EVT6-AML, TGS5, human telomerase reverse transcriptase, plysialic acid, intestinal carboxyl esterase, lewisY, sLe, LY6K, mut hsp70-2, M-CSF, RhoC, TRP-2, CYPIBI, BORIS, prostate-specific antigen (PSA), LAGE-la, NCAM, Ras mutant, gp100, prostein, OR51E2, PANX3, PSCA, HMWMAA, HAVCR1, VEGFR2, telomerase, legumain, sperm protein 17, SSEA-4, tyrosinase, TARP, prostate-carcinoma tumor antigen- 1 (PCTA-1), ML-IAP, MAD-CT-1, MAD-CT-2, MelanA/MART 1, XAGE1, ELF2M, NA17, neutrophil elastase, sarcoma translocation breakpoints, NY-BR-1, ephnnB2, androgen receptor, insulin growth factor (IGF)-I, IGF1, IGF-I receptor, GD2, o-acetyl-GD2, GD3, GM3, GPRC5D, GPR20, CXORF61, folate receptor (FRa), folate receptor beta, TAG72, TN Ag, Tie 2, TEM1, TEM7R, CLDN6, UPK2, mesothelin, BAGE proteins, CA9, CALR, CCR5, CD19, CD20 (MS4A1), CD22, CD24, CD27, CD30, CD33, CD38, CD40, CD44, CD52, CD56, CD79, Cd123, CD97, CD171, CD179a, CDK4, CEACAM3, CEACAM5, CLEC12A, DEPDC1, ERBB2 (HER2/neu), ERBB3, ERBB4, EPCAM, EPHA2, EPHA3, FCRL5, FOLR1, GAGE proteins, GPNMB, GPR112, IL3RA, LGR5, EBV-derived LMP2, LICAM, MAGE proteins, MAGE-A1, MLANA, MSLN, MUC1, MUC2, MUC3, MUC4, MUC5, MUC16, MUM1, ANKRD30A, NY-ESO1 (CTAGlB), OX40, PAP, PLAC1, PRLR, PMEL, PRAME, PSMA (FOLH1), RAGE proteins, RGS5, ROR1, ROS1, RU1, RU2, SART1, SART3, SLAMF7, SLC39A6 (LIV1), STEAP1, STEAP2, TMPRSS2, Thompson-nouvelle antigen, TNFRSF17, TYR, UPK3A, VTCN1, gp72, the ras oncogene product, HPV E6, HPV E7, beta-catenin, telomerase, melanoma gangliosides, ABL1, ABL2, AF15Q14, AFlQ, AF3p21, AF5q31, AKT, AKT2, ALK, ALO17, AML1, API, APC, ARHGEF, ARHH, ARNT, ASPSCR1, ATIC, ATM, AXL, BCL10, BCL11A, BCL11B, BCL2, BCL3, BCL5, BCL6, BCL7A, BCL9, BCR, BCR-ABL, BHD, BIRC3, BIRC5, BIRC7, BLM, BMPR1A, BRCA1, BRCA2, BRD4, BTG1, CBFA2T1, CBFA2T3, CBFB, CBL, CCND1, c-fos, CDH1, c-jun, CDK4, c-kit, CDKN2A- p14^{ARF}, CDKN2A - p16 ^{INK4A}, CDX2, CEBPA, CEP1, CHEK2, CHIC2, CHN1, CLTC, c-met, c-myc, COL1A1, COPEB, COX6C, CREBBP, c-ret, CTNNB1, CYLD, D10S170, DDB2, DDIT3, DDX10, DEK, EGFR, EIF4A2, ELKS, ELL, EP300, EPS15,, ERCC2, ERCC3, ERCC4, ERCC5, ERG, ETV1, ETV4, ETV6, EVI1, EWSR1, EXT1, EXT2, FACL6, FANCA, FANCC, FANCD2, FANCE, FANCF, FANCG, FEV, FGFR1, FGFR10P, FGFR2, FGFR3, FH, FIPlL1, FLI1, FLT3, FLT4, FMS, FNBP1, FOXO1A, FOXO3A, FPS, FSTL3, FUS, GAS7, GATA1, GIP, GMPS, GNAS, GOLGA5, GPC3, GPHN, GRAF, HEI10, HER3, HIP1, HIST1H4I, HLF, HMGA2, HOXA 11, HOXA13, HOXA9, HOXC13, HOXD11, HOXD13, HRAS, HRPT2, HSPCA, HSPCB, hTERT, IGHα, IGKα, IGLα, IL-11Ra, IL-13Ra, IL21R, IRF4, IRTA1, JAK2, KIT, KRAS, KRAS2, LAF4, LASP1, LCK, LCP1, LCX, LHFP, LMO1, LMO2, LPP, LYL1, MADH4, MALT1, MAML2, MAP2K4, MDM2, MECT1, MEN1, MET, MHC2TA, MLF1, MLHI1, MLL, MLLT1, MLLT10, MLLT2, MLLT3, MLLT4, MLLT6, MLLT7, MLM, MN1, MSF, MSH2, MSH6, MSN, MTSI1, MUTYH, MYC, MYCL1, MYCN, MYH11, MYH9, MYST4, NACA, NBS1, NCOA2, NCOA4, NFl, NF2, NOTCH1, NPM1, NR4A3, NRAS, NSDI, NTRK1, NTRK3, NUMA1, NUP214, NUP98, NUT, OLIG2, p53, mutant p53, p27, p57, p16, p21, p73, PAX3, PAX5, PAX7, PAX8, PBX1, PCM1, PDGFB, PDGFRA, PDGFRB, PICALM, PIM1, PML, PMS1, PMS2, PMX1, PNUTL1, POU2AF1, PPARG, PRAD-1, PRCC, PRKAR1A, PRO1073, PSIP2, PTCH, PTEN, PTPN11, RAB5EP, RAD51L1, RAF, RAP1GDS1, RARA, RAS, Rb, RB1, RECQL4, REL, RET, RPL22, RUNX1, RUNXBP2, SBDS, SDHB, SDHC, SDHD, SEPT6, SET, SFPQ, SH3GL1, SIS, SMAD2, SMAD3, SMAD4, SMARCB1, SMO, SRC, SS18, SS18L1, SSH3BP1, SSX1, SSX2, SSX4, Stathmin, STK11, STL, SUFU, TAF15, TAL1, TAL2, TCF1, TCF12, TCF3, TCL1A, TEC, TCF12, TFE3, TFEB, TFG, TFPT, TFRC, TIF1, TLX1, TLX3, TNFRSF6, TOP1, TP53, TPM3, TPM4, TPR, TRAα, TRBα, TRDα, TRIM33, TRIP11, TRK, TSC1, TSC2, TSHR, VHL, WAS, WHSC1L1 8, WRN, WT1, XPA, XPC, ZNF145, ZNF198, ZNF278, ZNF384, or ZNFN1A1.

It should be understood and herein contemplated that the methods described can detect more than one targets simultaneously, comprising adding multiple types of the biosensors described herein into a biological sample, wherein each type of the biosensors targets a nucleic acid that is different from the targeting nucleic acids of the other types of the biosensors. The methods can be used for detecting more than one type of pathogen and/or for diagnosing an infectious disease.

Accordingly, in some aspects, disclosed herein is a method of simultaneously detecting more than one type of pathogen in a subject, comprising
a) obtaining a biological sample from the subject; and
b) detecting more than one type of pathogen using more than one type of the biosensor disclosed herein, wherein each type of the biosensor comprises a latch strand that is capable of hybridizing to a target nucleic acid of one type of the pathogen.

### EXAMPLES

The following examples are set forth below to illustrate the compositions, methods, and results according to the disclosed subject matter. These examples are not intended to be inclusive of all aspects of the subject matter disclosed herein, but rather to illustrate representative methods and results. These examples are not intended to exclude equivalents and variations of the present invention which are apparent to one skilled in the art.

### Example 1 DNA Origami Hinges for COVID-19 Detection

As of mid-April 2020, the CDC’s preferred method of testing patients suspected to be infected by the COVID-19 virus is the real time reverse transcriptase polymerase chain reaction (RT-PCR) assay, designed to qualitatively detect the presence of the COVID-19 single-stranded RNA (ssRNA) genome from upper respiratory samples. This method, while accurate and indicative of current infection, is time consuming, labor intensive and requires significant materials. The current workflow for RT-PCR requires steps of sample collection, RNA extraction, RNA isolation, cDNA synthesis, qPCR, and readout. All these steps together can take hours to days. A more rapid method is the serological test for antibodies (15 to 20 minutes). However, the serological test fails to provide an accurate COVID-19 diagnosis from patients presenting an early to middle phase of infection or in cases involving immunodeficient individuals. Thus, developing an accurate COVID-19 positive assay confirming infection in a timely manner can be lifesaving.

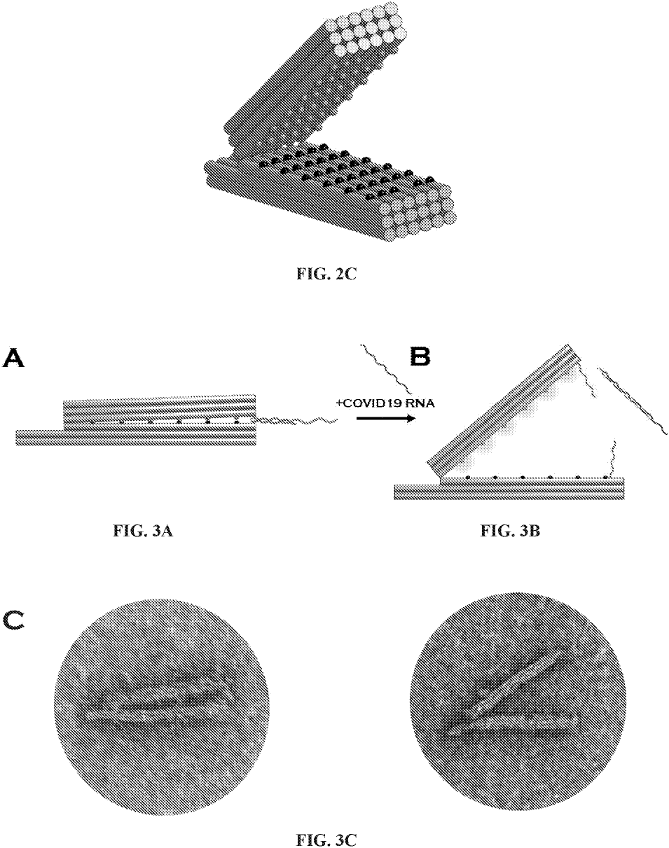

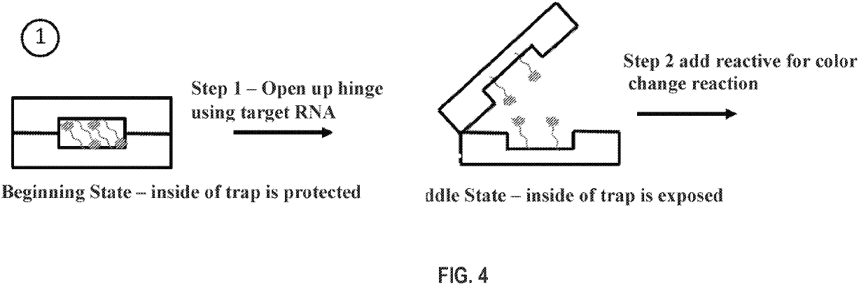

DNA origami is a nanotechnology that allows for detailed design and construction of 2D and 3D nanoscale objects and can combine different molecular functional items on a single nanostructure with unprecedented precision. A nanoscale hinge was constructed (FIGS. 2A-2C) and other devices that actuate (i.e. open or close) (FIGS. 3A-3C) upon binding of specific complementary oligonucleotides with conformational changes confirmed by fluorescent reporter, to allow bio-sensing applications, including specific ssRNA of the COVID-19 genome.

A rapid COVID-19 diagnostic assay employs the DNA origami “Hinge” nanostructure (FIGS. 3A-3C) to bind to an RNA sequence contained in the COVID-19 genome. This binding event causes a conformational change, “opening” the hinge and allowing the attached FAM fluorescent molecules to separate from the Black Hole quenchers (BHQ), and fluoresce (FIGS. 3A and 3B). To accomplish this, the top arm of the hinge is functionalized with FAM (FIG. 2C) and a ssRNA strand complementary to COVID-19 (FIGS. 3A-3B) including an unbound ‘toehold’ binding target sequence (FIG. 3A) and the bottom arm with BHQs and a fastening strand (FIG. 3A). Sequence specificity of DNA base-pairing is used to align the FAM and BHQ molecules. The ‘closed’ or ‘latched’ Hinge conformation (FIG. 3A) allows the BHQ and FAM to be within a few nanometers of each other, and thus allow only very low fluorescence emission occurs upon 488 nm excitation. COVID-19 ssRNA binding to complementary ssRNA strand ‘unlatches’ the Hinge arms thereby increasing the distance between the FAM and BHQ leading to a rapid and large increase in fluorescence emission (FIG. 3B). A major advantage of DNA origami over other nanotechnology platforms is the ability to precisely place a large number of aligned fluorescent molecules and quencher molecules along the arms, spaced at fixed distance intervals. Currently, the Hinge design allows for attachment of over 30 fluorophore and quencher pairs, which can yield a bright and easily detectable signal. Even more fluorophore-quencher pairs can be added, up to ~50, though cost and sensitivity dictates the design. Qualitative readouts of experiments include a fluorometer, plate reader, and thermocycler PCR machines with conformation change validation using agarose gel electrophoresis and TEM imaging.

The methods herein show positive correlation with RT-PCR testing procedures. In addition, the methods confirm the existence of low concentrations of COVID-19 RNA, which allows the full test to occur in less than 30 minutes. The sensitivity is improved by further increasing the number of FAM/BHQ pairs or Hinge device concentrations to increase the fluorescent readout. Alternatively, geometric shape design parameters can also be changed in a rapid manner.

DNA origami is a versatile tool that is emerging for use in biomedical applications, such as drug delivery, bio-sensing, and imaging. Similar to the Hinge device disclosed herein, many PCR systems use a combination of FAM fluorophores and Black Hole quenchers for an experimental readout, although they are generally limited to a single FAM fluorophore per (complementary) cDNA strand, which is copied from cDNA of the original RNA target (COVID-19). Alternatively, many fluorophores (up to 30 or more) can be attached on a single nanostructure enabling a bright signal without amplification of the COVID-19 cDNA, which saves time without altering the original readout. Recently, it was shown that fluorophores spaced within 2-5 nm display increased photons (over 6x) compared to a singular fluorophore, resulting in greater sensitivity. The DNA origami diagnostic test can be performed in about an hour with 40 minutes of that time using the same Viral RNA extraction kit that is used for current RT-PCR-based COVID-19 testing. The Hinge actuation occurs very rapidly, in seconds to minutes. Furthermore, the origami nanostructures disclosed herein have shown resistance to nuclease degradation, and the detection can be performed in cell lysate, which reduces the experimental procedural time to under 30 minutes. Additionally, the process requires only a simple addition of the DNA nanostructures to the RNA solution, allowing for the process to be performed with limited expertise and equipment.

The DNA origami hinge is roughly the same size as a virus (~100 nm). It can produce bright, glowing signal for each virus copy detected. The key design parts include the triggers for conformation changes and how the those occur.

DNA origami bio-sensing applications are not limited to the current COVID-19 pandemic, as changing the target sequence requires just a few oligonucleotide (out of 150+ in the total design) alterations to the DNA origami. This allows for adapting the technology to detect and diagnose many other DNA/RNA based viruses with only minor changes, and additionally can supplement some PCR functionalities. Furthermore, it was shown that DNA origami can be easily and quickly fabricated in large amounts (mg to g’s), with scaled-up manufacturing being cheap and attainable (estimates have origami produced for as low as ~$0.18 per mg). Thus, benefits of the technology include: 1) Faster than RT-PCR, can be performed in under 30 minutes; 2) Combines many fluorophores per nanostructure for an increased signal; 3) Uses similar readouts as existing technology and adaptable to other DNA/RNA based viruses; and 4) Fast, scalable fabrication.

### Example 2. DNA Origami Hinge Designs

Disclosed herein is a DNA-based nanoscale Biosensor constructed via the DNA origami molecular self-assembly process. The Biosensor is based on a hinge-like design consisting of two or more arms that are initially held in a closed configuration by a latching interaction. The latching interaction consists of nucleic acid base pairing between strands on distinct components, which are disrupted in the presence of a target nucleic acid sequence to convert the sensor into an open configuration. The latching interaction contains a section of the sequence that remains single stranded (ss) even in the closed configuration, referred to as a “toehold.” The target nucleic sequence causes the sensor to open by initially binding to the ssDNA or ssRNA toehold sequence and then competitively unbinds the latching sequence. This opening of the sensor reconfigures and/or exposes the functional molecules used for fluorescence, colorimetric, precipitation, or color changing readouts. Employing DNA origami Hinge-like devices as a platform for specific nucleic acid detection allows for a myriad of functionalities for this readout as a positive test for multiple disease states, such as RNA or DNA viral infections including SARS-CoV-2 (COVID-19). The device design includes the addition of fluorescent and quencher molecules on opposing arms of the ‘closed’ Hinge Biosensor. In the closed configuration, the quenchers are located near the fluorescent molecules, and when the sensor opens, the molecules separate leading to a strong increase in fluorescence. The DNA origami Hinge Biosensor can have many pairs of fluorophore/quenchers arranged in parallel so they “turn on” in unison to elicit an amplified signal (device contains about 45 pairs). This allows for the elimination of the time-consuming PCR or quantitative real time RT-PCR reactions that are common in the detection of viruses (including COVID-19) and other target nucleic acids. Additionally, Förster resonance energy transfer (FRET) can be used as a different signal output option.

A second version of the DNA origami Hinge Biosensor is designed with an internal cavity that contains catalytic molecules, which are occluded, or inaccessible, in the closed configuration (FIG. 2B). Hence, the molecules only react with the solution upon opening of the sensor by the target nucleic material. Additionally, the target can release the catalytic molecules or other functional readout material (e.g. horseradish peroxide with glucose oxidase for a colorimetric readout, iodine for reacting with a starch in a color changing reaction, polymer allowing for specific targeted precipitation, and gold nanoparticles) that can be attached inside the cavity include. The gold nanoparticles can be functionalized (e.g. with DNA) to aggregate when free in solution, thereby changing the solution color. The iodine and gold nanoparticles can be seen with the naked eye, allowing for on-site point-of-care diagnosis of a viral infection during a global pandemic.

A third version of the DNA origami Hinge Biosensor allows for the exposure of internal DNA sequences for two purposes. First, this is used as an amplification mechanism where strands that are exposed upon opening can trigger other devices so that one detection event can lead to a cascade of many devices fluorescing. This allows for detection of lower levels of target, even a single copy. Second, devices are designed where exposure of DNA sequences allows for higher order assembly of filaments or networks of the nanostructures. These networks can be detected by multiple possible methods. First, if they are large enough, they can precipitate and cause a cloudy solution. Second, staining with a DNA intercalator, or polymerizing with colored streptavidin/biotin, renders a visible change in color in the presence of large assemblies. Third, the assembly of networks can change the viscosity of the solution. Specific nucleic acid targets that the DNA origami Hinge Biosensor can detect include SARS-Cov-2 (also known as COVID-19), MERS, SARS and other viruses such as influenza. Furthermore, adding design features to any of the hinges, like cross-linking proximal thymidines by irradiating the nanostructures increases stability at high temperature, renders an increase in melting temperature and detection of double-stranded nucleotide targets. This increases the number of targets, including improved sensitivity in liquid biopsy detecting free circulating DNA, PCR-based experiments, or even loop-mediated isothermal amplification (LAMP).

The DNA origami designs provided herein comprises significant improvements over the prior designs. The challenges during processes of these improvements include, for example, increasing the number of overhang staple strand locations, lining up the fluorophore with the quenchers, hinge angle of the DNA origami “default” to be open configuration but still able to close hinge, latching mechanism that is strong enough to keep the DNA origami in a closed configuration but is still able to release and convert the DNA origami to an open configuration, the reuse mechanism, requirement of specific equipment for fluorescence readout, and optimal salt conditions.

The challenges are overcome by the improvements in design, including, for example, flattening the hinge, while still keeping stiff enough; changing the back section of design to change free body diagram; using “zipper” strand latch, which is about 100 bases complement to target nucleic acid; designing the zipper strand as not a part of DNA origami hinge structure such that DNA origami hinges can be “reloaded” with zipper strand; using trap design to protect from solution color changing molecules; and low enough to reduce secondary binding of Target/Zipper (internally), but still bind to structure. Patient samples come in various versions of transport media and contain a variety of protein, such as DNases and difference in cation concentration, that can have adverse effects on structure morphology and zipper binding, designing a structure with the optimal resilience in its stability is key. Using a square lattice and limiting the cross-overs in the design, in a similar fashion to previous designs, allows for a higher order of continuously bound segment sections before a holiday junction occurs. This improves the overall stability of the origami structure. In order to have the optimal quenching of the fluorophores, the attachment sites on each hinge arm must line up with their corresponding attachment site on the adjacent arm. Careful and deliberate structure design is necessary to ensure that the arms with line up and the fluorophores will be close enough to quench. Alternatively, to eliminate the need for equipment to read out, the Hinge Trap is designed to contain a protected molecule, that only changes color when in the open state, keeping the color-changing reaction apart when in the closed configuration. This technology is a rapid RNA-diagnostic based solution for quick and cheap testing (under 30 minutes) (estimates have DNA origami cost to be about $0.18 per mg in large scale production).

### Example 3. Latching Mechanisms

Definition of “Latch ”. The “latch” (also referred to as the “zipper” or “zipper strand”) is defined as the single stranded nucleic acid sequence that serves to: 1) close the “top arm” and “bottom arm” of the Hinge DNA origami nucleic acid biosensor 2) “sense” the intended nucleic acid target sequence of the DNA/RNA viral nucleic acid, or tumor specific DNA/RNA nucleic acid material and 3) respond to the intended nucleic acid target sequence via complementary base pair binding to cause the Hinge DNA origami nucleic acid biosensor to change from a “closed” to “open” configuration to allow for emission of a fluorescent, colorimetric, or precipitation signal readout.

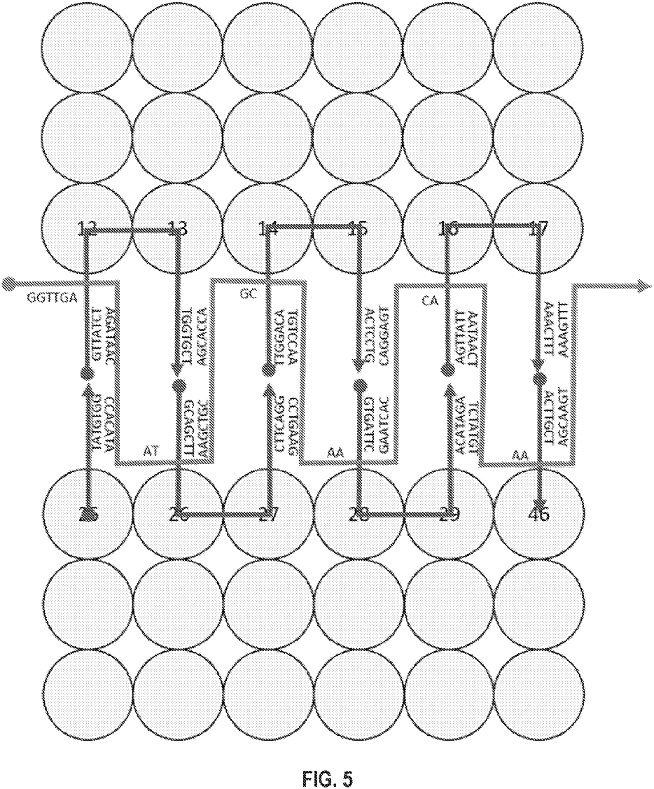

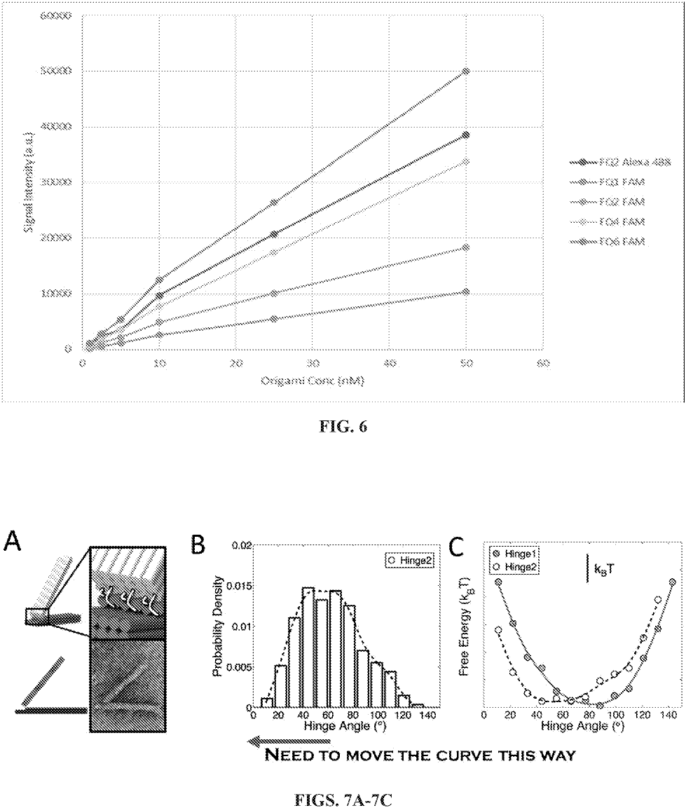

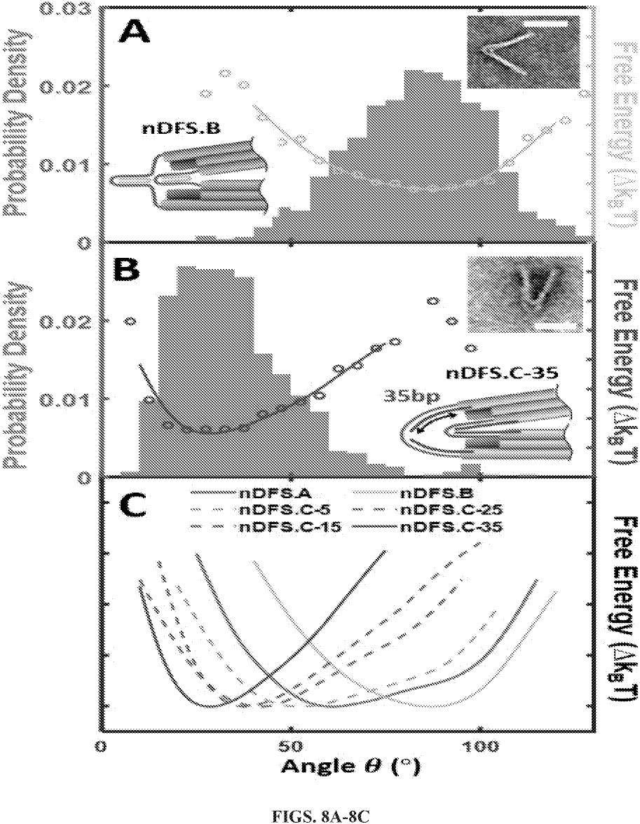

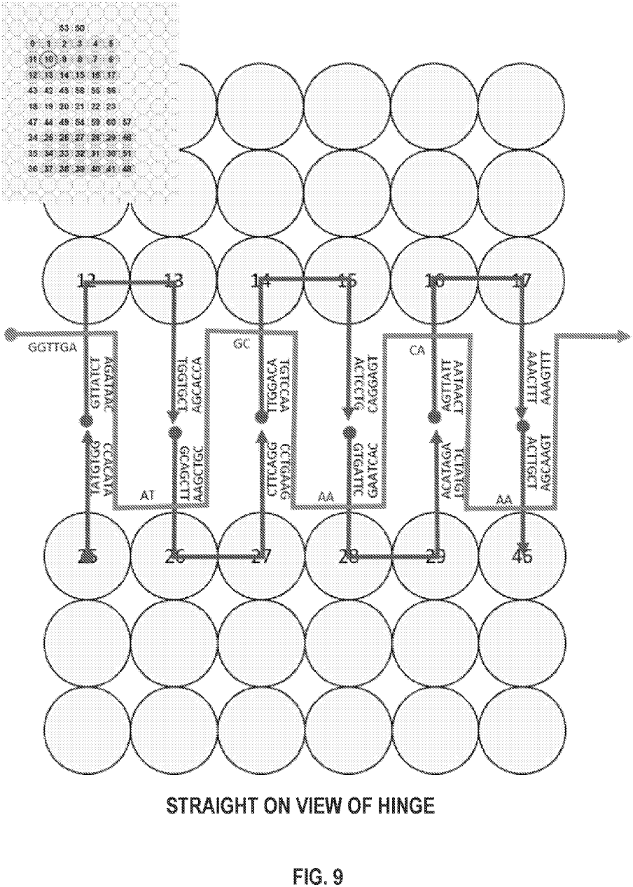

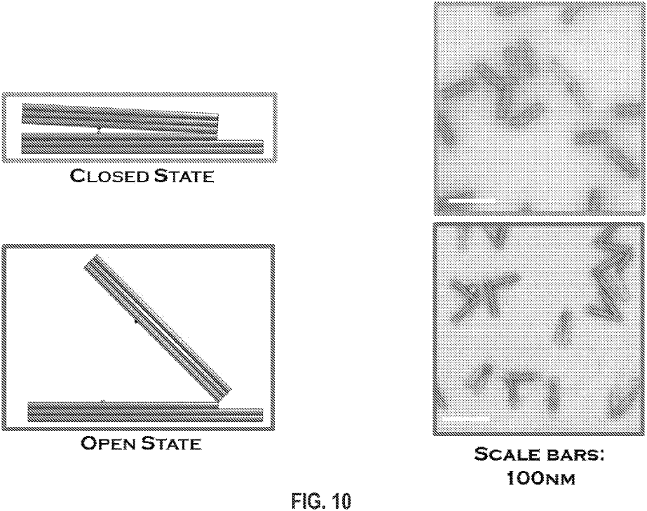

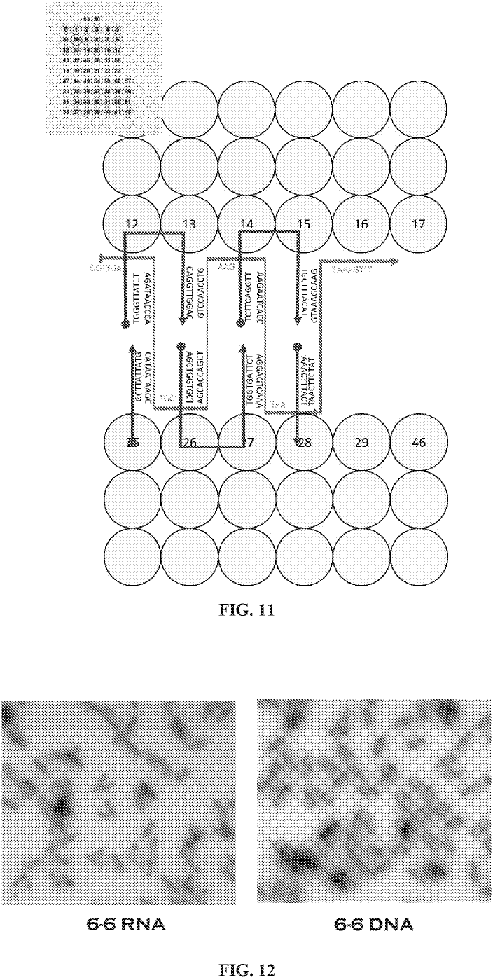

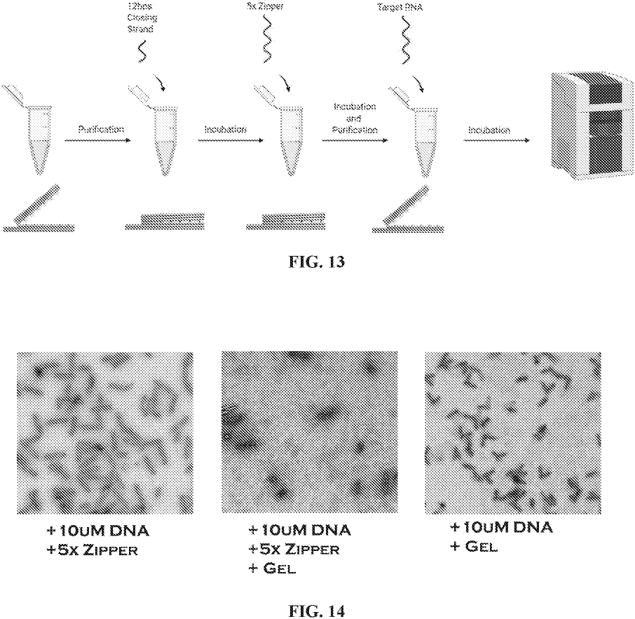

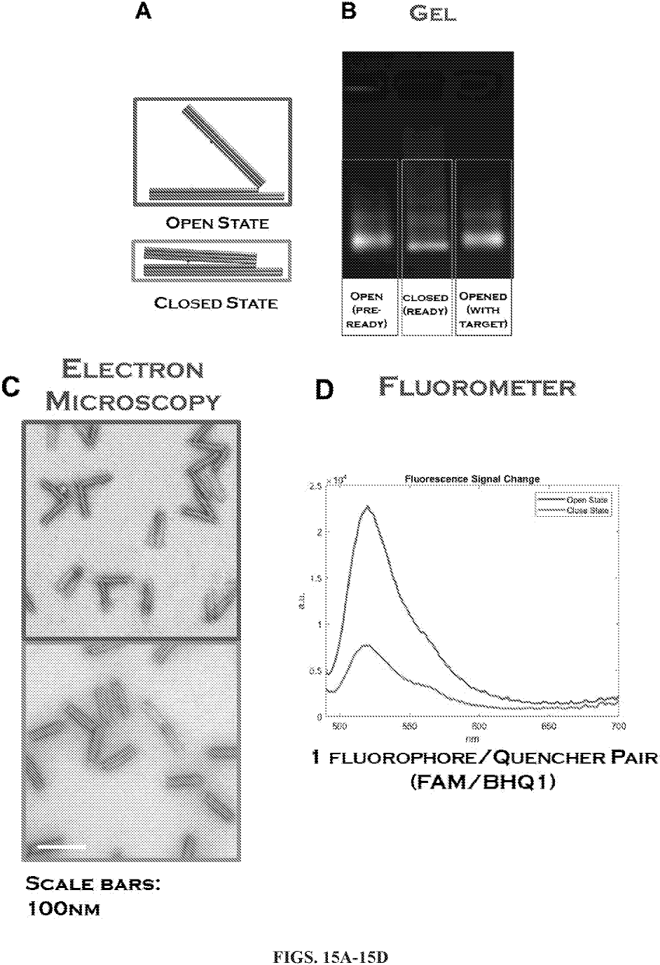

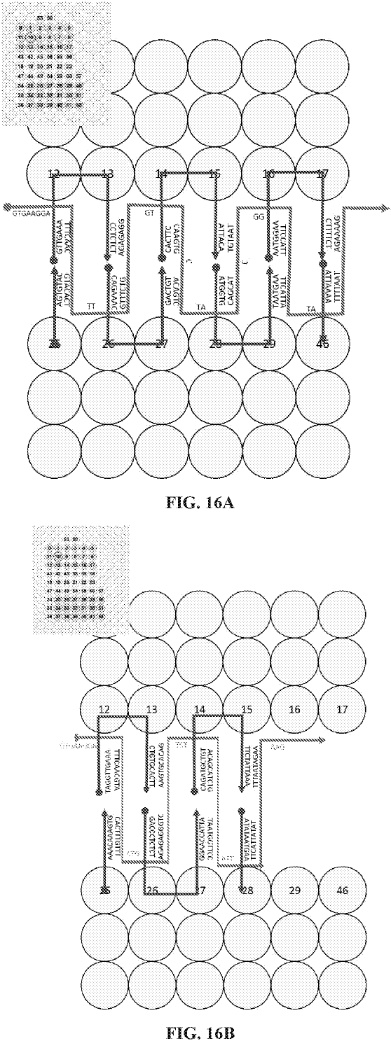

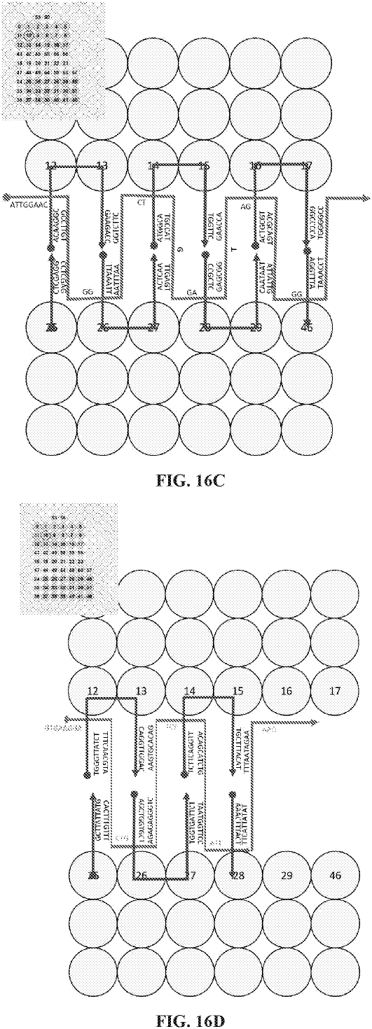

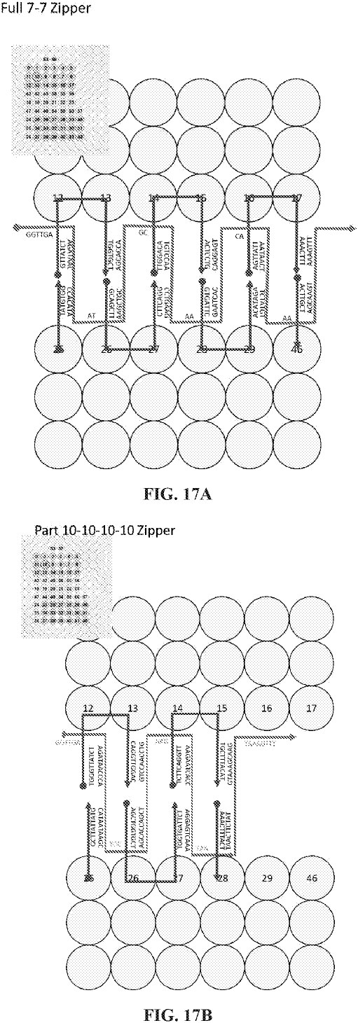

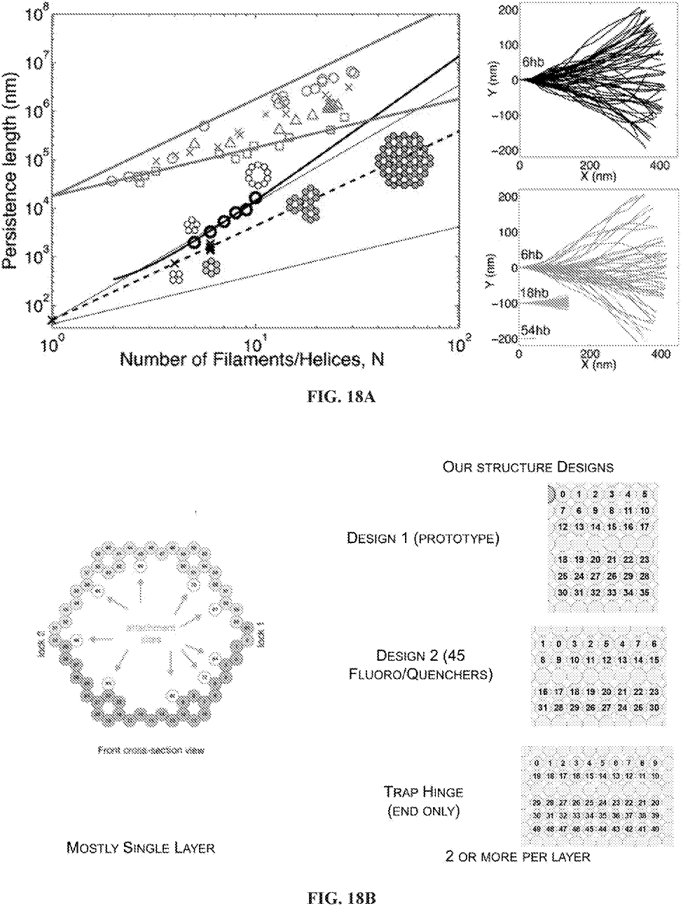

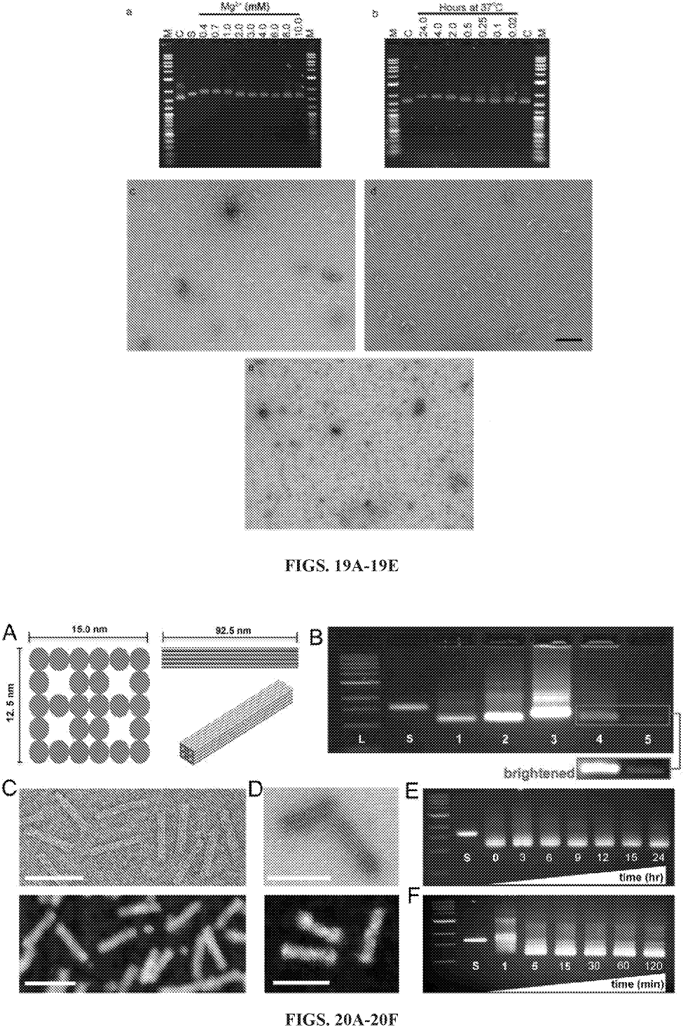

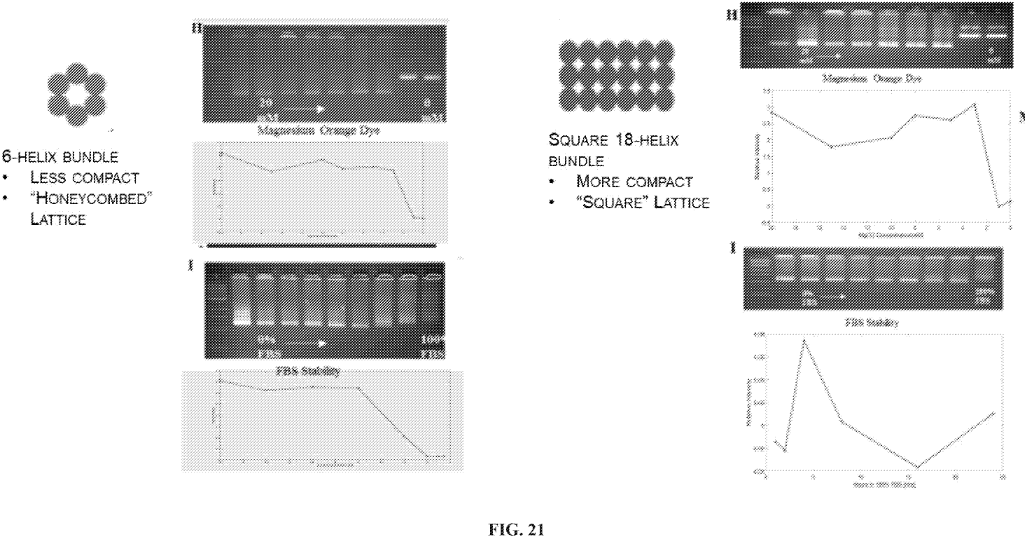

Composition and Mechanism of “Latch”. The “latch” consists of a single stranded ~100 base DNA or RNA oligonucleotide sequence designed with a potion with reverse complementarity (can be 100% identity) to the pre-defined target DNA/RNA viral or tumor nucleic acid sequence. The ‘latch’ oligonucleotide ‘closes’ the DNA origami Hinge Biosensor by base pairing with distinct regions of ‘overhang’ staple sequences that protrude from DNA helices from the ‘top arm’ and DNA helices from the ‘bottom arm’ of the Hinge DNA origami biosensor (se FIGS. 5, 17, 18). The 5′ and/or 3′ ends of the ‘latch’ strand (can be internal sequences) include free unbound sequences (6-20 bases in length) known as the ‘toehold’ sequence(s). The ‘toehold’ sequence(s) can facilitate toehold mediated strand displacement by the target viral or tumor DNA/RNA to release the ‘latch’ from the top and bottom arms of the Hinge DNA origami biosensor. This event allows the Hinge DNA origami biosensor to change from a ‘closed’ to ‘open’ configuration allowing for a fluorescence, colorimetric, or precipitation signal readout. In the case of SARS-CoV-2 viral detection, the ‘latch’ sequences are designed with a portion with 100% reverse complementarity to the specific regions of the ‘S’ and ‘N’ genes of the SARS-CoV-2 genome.

### Example 4 Rapid COVID-19 Diagnostic Testing Using DNA Origami Nanostructures

### 1. Purpose and Scope

Provided herein is a fluorescence-based DNA Origami COVID-19 diagnostic biosensor used in a clinical setting. Product iterations are designed to allow for a simple color change upon sensing SARS-CoV-2 target nucleic acid to facilitate rapid diagnostic testing at field point-of-care centers. Key steps toward product validation and de-risking include: 1) Effective DNA origami (DO) COVID-19 biosensor detection of viral nucleic acid in clinical samples; 2) Determining DO COVID-19 biosensor sensitivity in clinical samples (defining a lower limit of detection of viral nucleic acid material); 3) Evaluating long-term storage conditions and demonstrating capability to scale product production.

Demonstrate effective SARS-CoV-2 detection by DNA origami (DO) COVID-19 diagnostic biosensor in Clinical Samples: The DO COVID-19 biosensor design is optimized and evaluated/validated using i) long RNA target primers, ii) Research Use Only (RUO) SARS-CoV-2 viral material, and iii) COVID-19 clinical samples vs. healthy control samples. Outcomes include: 1) detection of 100 fM concentration of long SARS-CoV-2 RNA primer; 2) test negative against all non-target viral RNA and positive against SARS-CoV-2 RNA; 3) test negative for 100 out of 100 healthy patient samples, and test positive for 95 out of 100 COVID-19 patient samples.

Determine DO COVID-19 Diagnostic Biosensor Sensitivity in Clinical Samples: This study focuses on determining the biosensor limits of detection and detection variability in COVID-19 clinical samples. Outcomes include: 1) detection of 100 fM concentration of SARS-CoV-2 viral material; 2) low experimental variability between both replicate measurements and independently prepared batches of DO COVID-19 biosensors.

Quantify long-term storage stability and increased production of DO COVID-19 diagnostic biosensor: Stability of the DO COVID-19 diagnostic biosensor is tested under various storage conditions at various time points up to 6 months. Outcomes include: 1) optimal storage conditions are identified to preserve DO COVID-19 biosensor structural integrity, stability, and SARS-CoV-2 detection capabilities 2) scaling production process is established.

### 2. Tools and Materials.

The equipment necessary to produce and interpret diagnostic results, prior to testing detection, is provided by the clinical laboratory. This equipment includes:
Refrigeration equipment for sample storage
Pipettes and liquid handling instrumentation
Heating block tdeactivate samples (65° C. for 30 minutes)
Samples be frozen at -20° C. until transferred to testing laboratory.

Once testing laboratory receives samples, testing device is mixed with samples, heated to 37° C. for various time points, and read on a fluorometer instrument, real time PCR instrument, or a fluorescence based plate reader in the testing laboratory. All handling of de-identified patient samples are conducted in a BSL-2 bio-safety cabinet and in accordance with IBC protocol 202R00000058.

### 3. Procedure.

No deviations from these procedures are allowed without approval of the study sponsor (and the IRB/Ethics Committee if appropriate). Standard Precautions should be followed when working with clinical specimens and live microorganisms, including working in a biosafety cabinet. The tasks listed in this procedure must be performed by individuals who do not have knowledge of the test results.

### 3.1 Specimen Enrollment′ De-Identification

1. Use the following selection criteria to determine if a specimen is appropriate for enrollment: Inclusion Criteria:
Specimen is from a unique subject (i.e. a specimen from the subject has not been previously enrolled) and there is leftover specimen available.
Subject recruitment criteria:
   Any age
   Any gender
   Any medical condition

2. If a specimen qualifies for the study, record the name of the subject from whom the specimen was collected (or other appropriate subject identifier) on Appendix A: De-Identification Key. Also record the date and time the specimen was collected (as listed on the primary specimen container).

3. Assign the subject a number

4. Record the date of testing and results.

NOTE: Do not enroll specimens unless they appear to meet the inclusion criteria sufficient specimen volume is left over following diagnostic analysis as prescribed by the physician.

NOTE: The De-Identification Key should be the only link between the subject’s identification and the SCN assigned to the subject’s specimen. It should be maintained in accordance with IRB/EC-approved procedures and handled in compliance with local regulations.

### 3. 2. Sample Collection

Only leftover clinical samples that arrive to the clinical microbiology laboratory for COVID testing are being considered for this study. The samples are collected by qualified nurses and doctors following standard procedures. Only leftover samples are used for the study. Importantly, all leftover samples are heat inactivated on a heat block (65° C. for 30 minutes), and stored at -20° C. until sample transfer to testing laboratory.

### 3.3. Data Entry

Initial specimen information is entered. Enter other information including specimen type, collection date and testing, and results. Record the diagnostic call (Negative/Positive) as well as genotyping data if available.

### 3.4. Assay Overview and Methodology

Assay Overview. This experimental assay includes a ‘Hinge’ DNA origami nanostructure biosensor designed to detect low concentrations of SARS-CoV-2 RNA in solution. This biosensor can have 2-30 fluorescent molecules incorporated (paired with fluorescent quenching molecules), which only fluoresce when the target sequence RNA (SARS-CoV-2) is present.

The ‘Hinge’ DNA origami nanostructure is latched ‘closed’ by a ~100 base oligonucleotide that is the reverse complementary sequence to specific regions of the SARS-CoV-2 RNA genome. When the ‘Hinge’ DNA origami biosensor is mixed with a sample that contains SARS-CoV-2 RNA target sequences, the ‘Hinge’ DNA origami biosensor changes from a ‘closed’ to an ‘open’ configuration thus exposing fluorophore molecules that were quenched. Fluorescence can then be read on a real-time PCR instrument, a fluorometer, or a plate reader capable of fluorescence detection.

### Assay Methodology.

1.) Heat inactivated (65° C. for 30 minutes) deidentified clinical samples are obtained from the clinical laboratory by the testing laboratory (NBL) and kept frozen at -20° C. until use.

2.) Clinical samples are thawed in a BSL-2 biosafety cabinet and mixed with closed, open, or closed ‘off-target’ ‘Hinge’ DNA origami COVID-19 biosensors.

3.) Samples are heated at 37° C. for various time points (15-60 minutes).

4.) Sample fluorescence are measured in 3 different instruments including: 1) fluorometer 2) real time PCR instrument and 3) fluorescence-based plate reader.

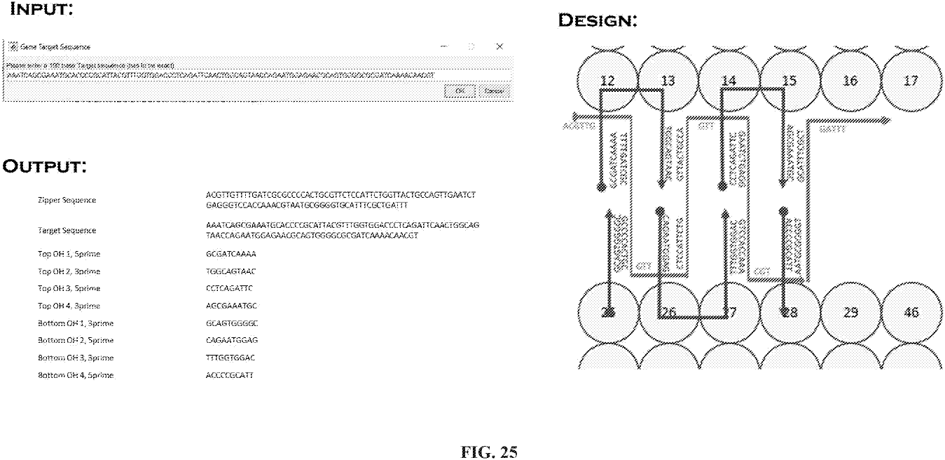

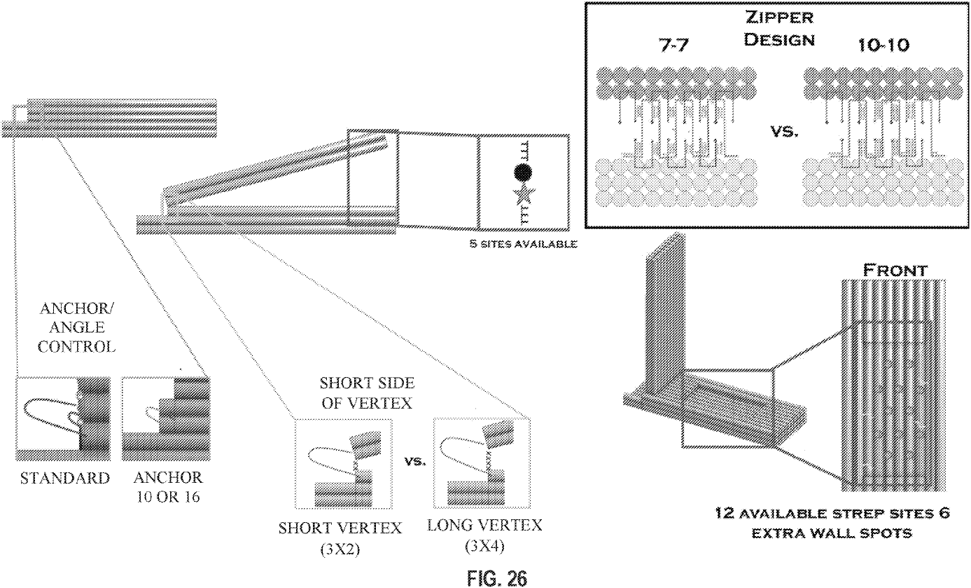

Custom Matlab Code Example. Written to design overhang sequences (for standard Hinge DNA origami biosensor 10-10 design). Example of input and output is shown in FIG. 25.

clc, clear all, close all
prompt = ’Please enter a 100 base Target sequence (has to be exact) ’;
dlgtitle = ’Gene Target Sequence’;
dims = [1 150];
define_input = {“};
tar_seq_cell = inputdig(prompt, digtitle, dims, define_input);
targ_seq = tar_seq_cel l { 1,1 };
zip_seq = seqrcomplement(targ_seq);
%% breaking up the zipper into the 10-10 configuration
Toeholdl = zip_seq(1:6);
TopOH_comp_1 = zip_seq(7:16);
BotOH_comp_1 = zip_seq(17:26);
Spacer_zip_1 = zip_seq(27:29);
BotOH_comp_2 = zip_seq(30:39);
TopOH_comp_2 = zip_seq(40:49);
Spacer_zip_2 = zip_seq(50:52);
TopOH_comp_3 = zip_seq(53:62);
BotOH_comp_3 = zip_seq(63:72);
Spacer_zip_3 = zip_seq(73:75);
BotOH_comp_4 = zip_seq(76:85);
TopOH_comp_4 = zip_seq(86:95);
Toehold2 = zip_seq(96:100);
%% Flipping the sequences for the Overhang side
TopOH_1 = seqrcomplement(TopOH_comp_1);
BotOH_1 = seqrcomplement(BotOH_comp_1);
BotOH_2 = seqrcomplement(BotOH_comp_2);
TopOH_2 = seqrcomplement(TopOH_comp_2);
TopOH_3 = seqrcomplement(TopOH_comp_3);
BotOH_3 = seqrcomplement(BotOH_comp_3);
BotOH_4 = seqrcomplement(BotOH_comp_4);
TopOH 4 = seqrcomplement(TopOH_com_4);
Sequence_data = {’Zipper Sequence’, zip_seq;’Target Sequence’, targ_seq; “Top OH 1,
5prime’,TopOH_1;’Top OH 2, 3prime’, TopOH_2;’Top OH 3, 5prime’, TopOH_3;’Top OH 4,
3prime’, TopOH_4;’Bottom OH 1, 3prime’,BotOH_1;’Bottom OH 2, 5prime’,
BotOH_2;’Bottom OH 3, 3prime’, BotOH_3;’Bottom OH 4, 5prime’, BotOH_4};
%% Sending Ordering Sequences to Excel
xlswrite(’Sequence info.xls’,Sequence_data)

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will appreciate that numerous changes and modifications can be made to the preferred embodiments of the invention and that such changes and modifications can be made without departing from the spirit of the invention. It is, therefore, intended that the appended claims cover all such equivalent variations as fall within the true spirit and scope of the invention.

### SEQUENCES

SEQ ID NO: 1 (sequence of scaffold strand, M13mp18 bacteriophage genome (7249 bases in length plus an 815 base insert sequence, total length = 8064 bases)

TGATAGACGGTTTTTCGCCCTTTGACGTTGGAGTCCACGTTCTTT
AATAGTGGACTCTTGTTCCAAACTGGAACAACACTCAACCCTATCTCGGG
CTATTCTTTTGATTTATAAGGGATTTTGCCGATTTCGGAACCACCATCAA
ACAGGATTTTCGCCTGCTGGGGCAAACCAGCGTGGACCGCTTGCTGCAAC
TCTCTCAGGGCCAGGCGGTGAAGGGCAATCAGCTGTTGCCCGTCTCACTG
GTGAAAAGAAAAACCACCCTGGCGCCCAATACGCAAACCGCCTCTCCCCG
CGCGTTGGCCGATTCATTAATGCAGCTGGCACGACAGGTTTCCCGACTGG
AAAGCGGGCAGTGAGCGCAACGCAATTAATGTGAGTTAGCTCACTCATTA
GGCACCCCAGGCTTTACACTTTATGCTTCCGGCTCGTATGTTGTGTGGAA
TTGTGAGCGGATAACAATTTCACACAGGAAACAGCTATGACCATGATTAC
GAATTCGAGCTCGGTACCCGGGGATCCTCAACTGTGAGGAGGCTCACGGA
CGCGAAGAACAGGCACGCGTGCTGGCAGAAACCCCCGGTATGACCGTGAA
AACGGCCCGCCGCATTCTGGCCGCAGCACCACAGAGTGCACAGGCGCGCA
GTGACACTGCGCTGGATCGTCTGATGCAGGGGGCACCGGCACCGCTGGCT
GCAGGTAACCCGGCATCTGATGCCGTTAACGATTTGCTGAACACACCAGT
GTAAGGGATGTTTATGACGAGCAAAGAAACCTTTACCCATTACCAGCCGC
AGGGCAACAGTGACCCGGCTCATACCGCAACCGCGCCCGGCGGATTGAGT
GCGAAAGCGCCTGCAATGACCCCGCTGATGCTGGACACCTCCAGCCGTAA
GCTGGTTGCGTGGGATGGCACCACCGACGGTGCTGCCGTTGGCATTCTTG
CGGTTGCTGCTGACCAGACCAGCACCACGCTGACGTTCTACAAGTCCGGC
ACGTTCCGTTATGAGGATGTGCTCTGGCCGGAGGCTGCCAGCGACGAGAC
GAAAAAACGGACCGCGTTTGCCGGAACGGCAATCAGCATCGTTTAACTTT
ACCCTTCATCACTAAAGGCCGCCTGTGCGGCTTTTTTTACGGGATTTTTT
TATGTCGATGTACACAACCGCCCAACTGCTGGCGGCAAATGAGCAGAAAT
TTAAGTTTGATCCGCTGTTTCTGCGTCTCTTTTTCCGTGAGAGCTATCCC
TTCACCACGGAGAAAGTCTATCTCTCACAAATTCCGGGACTGGTAAACAT
GGCGCTGTACGTTTCGCCGATTGTTTCCGGTGAGGTTATCCGTTCCCGTG
GCGGCTCCACCTCTGAAAGCTTGGCACTGGCCGTCGTTTTACAACGTCGT
GACTGGGAAAACCCTGGCGTTACCCAACTTAATCGCCTTGCAGCACATCC
CCCTTTCGCCAGCTGGCGTAATAGCGAAGAGGCCCGCACCGATCGCCCTT
CCCAACAGTTGCGCAGCCTGAATGGCGAATGGCGCTTTGCCTGGTTTCCG
GCACCAGAAGCGGTGCCGGAAAGCTGGCTGGAGTGCGATCTTCCTGAGGC
CGATACTGTCGTCGTCCCCTCAAACTGGCAGATGCACGGTTACGATGCGC
CCATCTACACCAACGTGACCTATCCCATTACGGTCAATCCGCCGTTTGTT
CCCACGGAGAATCCGACGGGTTGTTACTCGCTCACATTTAATGTTGATGA
AAGCTGGCTACAGGAAGGCCAGACGCGAATTATTTTTGATGGCGTTCCTA
TTGGTTAAAAAATGAGCTGATTTAACAAAAATTTAATGCGAATTTTAACA
AAATATTAACGTTTACAATTTAAATATTTGCTTATACAATCTTCCTGTTT
TTGGGGCTTTTCTGATTATCAACCGGGGTACATATGATTGACATGCTAGT
TTTACGATTACCGTTCATCGATTCTCTTGTTTGCTCCAGACTCTCAGGCA
ATGACCTGATAGCCTTTGTAGATCTCTCAAAAATAGCTACCCTCTCCGGC
ATTAATTTATCAGCTAGAACGGTTGAATATCATATTGATGGTGATTTGAC
TGTCTCCGGCCTTTCTCACCCTTTTGAATCTTTACCTACACATTACTCAG
GCATTGCATTTAAAATATATGAGGGTTCTAAAAATTTTTATCCTTGCGTT
GAAATAAAGGCTTCTCCCGCAAAAGTATTACAGGGTCATAATGTTTTTGG
TACAACCGATTTAGCTTTATGCTCTGAGGCTTTATTGCTTAATTTTGCTA
ATTCTTTGCCTTGCCTGTATGATTTATTGGATGTTAATGCTACTACTATT
AGTAGAATTGATGCCACCTTTTCAGCTCGCGCCCCAAATGAAAATATAGC
TAAACAGGTTATTGACCATTTGCGAAATGTATCTAATGGTCAAACTAAAT
CTACTCGTTCGCAGAATTGGGAATCAACTGTTATATGGAATGAAACTTCC
AGACACCGTACTTTAGTTGCATATTTAAAACATGTTGAGCTACAGCATTA
TATTCAGCAATTAAGCTCTAAGCCATCCGCAAAAATGACCTCTTATCAAA
AGGAGCAATTAAAGGTACTCTCTAATCCTGACCTGTTGGAGTTTGCTTCC
GGTCTGGTTCGCTTTGAAGCTCGAATTAAAACGCGATATTTGAAGTCTTT
CGGGCTTCCTCTTAATCTTTTTGATGCAATCCGCTTTGCTTCTGACTATA
ATAGTCAGGGTAAAGACCTGATTTTTGATTTATGGTCATTCTCGTTTTCT
GAACTGTTTAAAGCATTTGAGGGGGATTCAATGAATATTTATGACGATTC
CGCAGTATTGGACGCTATCCAGTCTAAACATTTTACTATTACCCCCTCTG
GCAAAACTTCTTTTGCAAAAGCCTCTCGCTATTTTGGTTTTTATCGTCGT
CTGGTAAACGAGGGTTATGATAGTGTTGCTCTTACTATGCCTCGTAATTC
CTTTTGGCGTTATGTATCTGCATTAGTTGAATGTGGTATTCCTAAATCTC
AACTGATGAATCTTTCTACCTGTAATAATGTTGTTCCGTTAGTTCGTTTT
ATTAACGTAGATTTTTCTTCCCAACGTCCTGACTGGTATAATGAGCCAGT
TCTTAAAATCGCATAAGGTAATTCACAATGATTAAAGTTGAAATTAAACC
ATCTCAAGCCCAATTTACTACTCGTTCTGGTGTTTCTCGTCAGGGCAAGC
CTTATTCACTGAATGAGCAGCTTTGTTACGTTGATTTGGGTAATGAATAT
CCGGTTCTTGTCAAGATTACTCTTGATGAAGGTCAGCCAGCCTATGCGCC
TGGTCTGTACACCGTTCATCTGTCCTCTTTCAAAGTTGGTCAGTTCGGTT
CCCTTATGATTGACCGTCTGCGCCTCGTTCCGGCTAAGTAACATGGAGCA
GGTCGCGGATTTCGACACAATTTATCAGGCGATGATACAAATCTCCGTTG
TACTTTGTTTCGCGCTTGGTATAATCGCTGGGGGTCAAAGATGAGTGTTT
TAGTGTATTCTTTTGCCTCTTTCGTTTTAGGTTGGTGCCTTCGTAGTGGC
ATTACGTATTTTACCCGTTTAATGGAAACTTCCTCATGAAAAAGTCTTTA
GTCCTCAAAGCCTCTGTAGCCGTTGCTACCCTCGTTCCGATGCTGTCTTT
CGCTGCTGAGGGTGACGATCCCGCAAAAGCGGCCTTTAACTCCCTGCAAG
CCTCAGCGACCGAATATATCGGTTATGCGTGGGCGATGGTTGTTGTCATT
GTCGGCGCAACTATCGGTATCAAGCTGTTTAAGAAATTCACCTCGAAAGC
AAGCTGATAAACCGATACAATTAAAGGCTCCTTTTGGAGCCTTTTTTTTG
GAGATTTTCAACGTGAAAAAATTATTATTCGCAATTCCTTTAGTTGTTCC
TTTCTATTCTCACTCCGCTGAAACTGTTGAAAGTTGTTTAGCAAAATCCC
ATACAGAAAATTCATTTACTAACGTCTGGAAAGACGACAAAACTTTAGAT
CGTTACGCTAACTATGAGGGCTGTCTGTGGAATGCTACAGGCGTTGTAGT
TTGTACTGGTGACGAAACTCAGTGTTACGGTACATGGGTTCCTATTGGGC
TTGCTATCCCTGAAAATGAGGGTGGTGGCTCTGAGGGTGGCGGTTCTGAG
GGTGGCGGTTCTGAGGGTGGCGGTACTAAACCTCCTGAGTACGGTGATAC
ACCTATTCCGGGCTATACTTATATCAACCCTCTCGACGGCACTTATCCGC
CTGGTACTGAGCAAAACCCCGCTAATCCTAATCCTTCTCTTGAGGAGTCT
CAGCCTCTTAATACTTTCATGTTTCAGAATAATAGGTTCCGAAATAGGCA
GGGGGCATTAACTGTTTATACGGGCACTGTTACTCAAGGCACTGACCCCG
TTAAAACTTATTACCAGTACACTCCTGTATCATCAAAAGCCATGTATGAC
GCTTACTGGAACGGTAAATTCAGAGACTGCGCTTTCCATTCTGGCTTTAA
TGAGGATTTATTTGTTTGTGAATATCAAGGCCAATCGTCTGACCTGCCTC
AACCTCCTGTCAATGCTGGCGGCGGCTCTGGTGGTGGTTCTGGTGGCGGC
TCTGAGGGTGGTGGCTCTGAGGGTGGCGGTTCTGAGGGTGGCGGCTCTGA
GGGAGGCGGTTCCGGTGGTGGCTCTGGTTCCGGTGATTTTGATTATGAAA
AGATGGCAAACGCTAATAAGGGGGCTATGACCGAAAATGCCGATGAAAAC
GCGCTACAGTCTGACGCTAAAGGCAAACTTGATTCTGTCGCTACTGATTA
CGGTGCTGCTATCGATGGTTTCATTGGTGACGTTTCCGGCCTTGCTAATG
GTAATGGTGCTACTGGTGATTTTGCTGGCTCTAATTCCCAAATGGCTCAA
GTCGGTGACGGTGATAATTCACCTTTAATGAATAATTTCCGTCAATATTT
ACCTTCCCTCCCTCAATCGGTTGAATGTCGCCCTTTTGTCTTTGGCGCTG
GTAAACCATATGAATTTTCTATTGATTGTGACAAAATAAACTTATTCCGT
GGTGTCTTTGCGTTTCTTTTATATGTTGCCACCTTTATGTATGTATTTTC
TACGTTTGCTAACATACTGCGTAATAAGGAGTCTTAATCATGCCAGTTCT
TTTGGGTATTCCGTTATTATTGCGTTTCCTCGGTTTCCTTCTGGTAACTT
TGTTCGGCTATCTGCTTACTTTTCTTAAAAAGGGCTTCGGTAAGATAGCT
ATTGCTATTTCATTGTTTCTTGCTCTTATTATTGGGCTTAACTCAATTCT
TGTGGGTTATCTCTCTGATATTAGCGCTCAATTACCCTCTGACTTTGTTC
AGGGTGTTCAGTTAATTCTCCCGTCTAATGCGCTTCCCTGTTTTTATGTT
ATTCTCTCTGTAAAGGCTGCTATTTTCATTTTTGACGTTAAACAAAAAAT
CGTTTCTTATTTGGATTGGGATAAATAATATGGCTGTTTATTTTGTAACT
GGCAAATTAGGCTCTGGAAAGACGCTCGTTAGCGTTGGTAAGATTCAGGA
TAAAATTGTAGCTGGGTGCAAAATAGCAACTAATCTTGATTTAAGGCTTC
AAAACCTCCCGCAAGTCGGGAGGTTCGCTAAAACGCCTCGCGTTCTTAGA
ATACCGGATAAGCCTTCTATATCTGATTTGCTTGCTATTGGGCGCGGTAA
TGATTCCTACGATGAAAATAAAAACGGCTTGCTTGTTCTCGATGAGTGCG
GTACTTGGTTTAATACCCGTTCTTGGAATGATAAGGAAAGACAGCCGATT
ATTGATTGGTTTCTACATGCTCGTAAATTAGGATGGGATATTATTTTTCT
TGTTCAGGACTTATCTATTGTTGATAAACAGGCGCGTTCTGCATTAGCTG
AACATGTTGTTTATTGTCGTCGTCTGGACAGAATTACTTTACCTTTTGTC
GGTACTTTATATTCTCTTATTACTGGCTCGAAAATGCCTCTGCCTAAATT
ACATGTTGGCGTTGTTAAATATGGCGATTCTCAATTAAGCCCTACTGTTG
AGCGTTGGCTTTATACTGGTAAGAATTTGTATAACGCATATGATACTAAA
CAGGCTTTTTCTAGTAATTATGATTCCGGTGTTTATTCTTATTTAACGCC
TTATTTATCACACGGTCGGTATTTCAAACCATTAAATTTAGGTCAGAAGA
TGAAATTAACTAAAATATATTTGAAAAAGTTTTCTCGCGTTCTTTGTCTT
GCGATTGGATTTGCATCAGCATTTACATATAGTTATATAACCCAACCTAA
GCCGGAGGTTAAAAAGGTAGTCTCTCAGACCTATGATTTTGATAAATTCA
CTATTGACTCTTCTCAGCGTCTTAATCTAAGCTATCGCTATGTTTTCAAG
GATTCTAAGGGAAAATTAATTAATAGCGACGATTTACAGAAGCAAGGTTA
TTCACTCACATATATTGATTTATGTACTGTTTCCATTAAAAAAGGTAATT
CAAATGAAATTGTTAAATGTAATTAATTTTGTTTTCTTGATGTTTGTTTC
ATCATCTTCTTTTGCTCAGGTAATTGAAATGAATAATTCGCCTCTGCGCG
ATTTTGTAACTTGGTATTCAAAGCAATCAGGCGAATCCGTTATTGTTTCT
CCCGATGTAAAAGGTACTGTTACTGTATATTCATCTGACGTTAAACCTGA
AAATCTACGCAATTTCTTTATTTCTGTTTTACGTGCAAATAATTTTGATA
TGGTAGGTTCTAACCCTTCCATTATTCAGAAGTATAATCCAAACAATCAG
GATTATATTGATGAATTGCCATCATCTGATAATCAGGAATATGATGATAA
TTCCGCTCCTTCTGGTGGTTTCTTTGTTCCGCAAAATGATAATGTTACTC
AAACTTTTAAAATTAATAACGTTCGGGCAAAGGATTTAATACGAGTTGTC
GAATTGTTTGTAAAGTCTAATACTTCTAAATCCTCAAATGTATTATCTAT
TGACGGCTCTAATCTATTAGTTGTTAGTGCTCCTAAAGATATTTTAGATA
ACCTTCCTCAATTCCTTTCAACTGTTGATTTGCCAACTGACCAGATATTG
ATTGAGGGTTTGATATTTGAGGTTCAGCAAGGTGATGCTTTAGATTTTTC
ATTTGCTGCTGGCTCTCAGCGTGGCACTGTTGCAGGCGGTGTTAATACTG
ACCGCCTCACCTCTGTTTTATCTTCTGCTGGTGGTTCGTTCGGTATTTTT
AATGGCGATGTTTTAGGGCTATCAGTTCGCGCATTAAAGACTAATAGCCA
TTCAAAAATATTGTCTGTGCCACGTATTCTTACGCTTTCAGGTCAGAAGG
GTTCTATCTCTGTTGGCCAGAATGTCCCTTTTATTACTGGTCGTGTGACT
GGTGAATCTGCCAATGTAAATAATCCATTTCAGACGATTGAGCGTCAAAA
TGTAGGTATTTCCATGAGCGTTTTTCCTGTTGCAATGGCTGGCGGTAATA
TTGTTCTGGATATTACCAGCAAGGCCGATAGTTTGAGTTCTTCTACTCAG
GCAAGTGATGTTATTACTAATCAAAGAAGTATTGCTACAACGGTTAATTT
GCGTGATGGACAGACTCTTTTACTCGGTGGCCTCACTGATTATAAAAACA
CTTCTCAGGATTCTGGCGTACCGTTCCTGTCTAAAATCCCTTTAATCGGC
CTCCTGTTTAGCTCCCGCTCTGATTCTAACGAGGAAAGCACGTTATACGT
GCTCGTCAAAGCAACCATAGTACGCGCCCTGTAGCGGCGCATTAAGCGCG
GCGGGTGTGGTGGTTACGCGCAGCGTGACCGCTACACTTGCCAGCGCCCT
AGCGCCCGCTCCTTTCGCTTTCTTCCCTTCCTTTCTCGCCACGTTCGCCG
GCTTTCCCCGTCAAGCTCTAAATCGGGGGCTCCCTTTAGGGTTCCGATTT
AGTGCTTTACGGCACCTCGACCCCAAAAAACTTGATTTGGGTGATGGTTC
ACGTAGTGGGCCATCGCCC

SEQ ID NO: 2 (fastening sequence of overhang stapl
e strand for 7-7 zipper, top) GTTATCT

SEQ ID NO: 3 (fastening sequence of overhang stapl
e strand for 7-7 zipper, top) TGGTGCT

SEQ ID NO: 4 (fastening sequence of overhang stapl
e strand for 7-7 zipper, top) TTGGACA

SEQ ID NO: 5 (fastening sequence of overhang stapl
e strand for 7-7 zipper, top) ACTCCTG

SEQ ID NO: 6 (fastening sequence of overhang stapl
e strand for 7-7 zipper, top) AGTTATT

SEQ ID NO: 7 (fastening sequence of overhang stapl
e strand for 7-7 zipper, top) AAACTTT

SEQ ID NO: 8 (fastening sequence of overhang stapl
e strand for 7-7 zipper, bottom) TATGTGG

SEQ ID NO: 9 (fastening sequence of overhang stapl
e strand for 7-7 zipper, bottom) GCAGCTT

SEQ ID NO: 10 (fastening sequence of overhang stap
le strand for 7-7 zipper, bottom) CTTCAGG

SEQ ID NO: 11 (fastening sequence of overhang stap
le strand for 7-7 zipper, bottom) GTGATTC

SEQ ID NO: 12 (fastening sequence of overhang stap
le strand for 7-7 zipper, bottom) ACATAGA

SEQ ID NO: 13 (fastening sequence of overhang stap
le strand for 7-7 zipper, bottom) ACTTGCT

SEQ ID NO: 14 (fastening sequence of overhang stap
le strand for 10-10 zipper, top) TGGGTTATCT

SEQ ID NO: 15 (fastening sequence of overhang stap
le strand for 10-10 zipper, top) CAGGTTGGAC

SEQ ID NO: 16 (fastening sequence of overhang stap
le strand for 10-10 zipper, top) TCTTCAGGTT

SEQ ID NO: 17 (fastening sequence of overhang stap
le strand for 10-10 zipper, top) TGCTTTACAT

SEQ ID NO: 18 (fastening sequence of overhang stap
le strand for 10-10 zipper, bottom) GCTTATTAT
G

SEQ ID NO: 19 (fastening sequence of overhang stap
le strand for 10-10 zipper, bottom) AGCTGGTGCT

SEQ ID NO: 20 (fastening sequence of overhang stap
le strand for 10-10 zipper, bottom) TGGTGATTC
T

SEQ ID NO: 21 (fastening sequence of overhang stap
le strand for 10-10 zipper, bottom) AAACTTTAC
T

SEQ ID NO: 22 (fastening sequence of overhang stap
le strand for 7-7 zipper, top) GTTGAAA

SEQ ID NO: 23 (fastening sequence of overhang stap
le strand for 7-7 zipper, top) CCTCTCT

SEQ ID NO: 24 (fastening sequence of overhang stap
le strand for 7-7 zipper, top) CACTTC

SEQ ID NO: 25 (fastening sequence of overhang stap
le strand for 7-7 zipper, top) ATTACA

SEQ ID NO: 26 (fastening sequence of overhang stap
le strand for 7-7 zipper, top) AATGGAA

SEQ ID NO: 27 (fastening sequence of overhang stap
le strand for 7-7 zipper, top) CTTTTCT

SEQ ID NO: 28 (fastening sequence of overhang stap
le strand for 7-7 zipper, bottom) AGTGTAC

SEQ ID NO: 29 (fastening sequence of overhang stap
le strand for 7-7 zipper, bottom) CAGAAAC

SEQ ID NO: 30 (fastening sequence of overhang stap
le strand for 7-7 zipper, bottom) GACTGT

SEQ ID NO: 31 (fastening sequence of overhang stap
le strand for 7-7 zipper, bottom) ATGGTG

SEQ ID NO: 32 (fastening sequence of overhang stap
le strand for 7-7 zipper, bottom) TAATGAA

SEQ ID NO: 33 (fastening sequence of overhang stap
le strand for 7-7 zipper, bottom) ATTAAAA

SEQ ID NO: 34 (fastening sequence of overhang stap
le strand for 10-10 zipper, top) TAGGTTGAAA

SEQ ID NO: 35 (fastening sequence of overhang stap
le strand for 10-10 zipper, top) CTGTGCACTT

SEQ ID NO: 36 (fastening sequence of overhang stap
le strand for 10-10 zipper, top) CAGATGCTGT

SEQ ID NO: 37 (fastening sequence of overhang stap
le strand for 10-10 zipper, top) TTCTATTAAA

SEQ ID NO: 38 (fastening sequence of overhang stap
le strand for 10-10 zipper, bottom) AAACAAAGT
G

SEQ ID NO: 39 (fastening sequence of overhang stap
le strand for 10-10 zipper, bottom) GACCCTCTC
T

SEQ ID NO: 40 (fastening sequence of overhang stap
le strand for 10-10 zipper, bottom) GGAACCATT
A

SEQ ID NO: 41 (fastening sequence of overhang stap
le strand for 10-10 zipper, bottom) ATATAATGA
A

SEQ ID NO: 42 (fastening sequence of overhang stap
le strand for 7-7 zipper, top) ACAAGGC

SEQ ID NO: 43 (fastening sequence of overhang stap
le strand for 7-7 zipper, top) GAAGACC

SEQ ID NO: 44 (fastening sequence of overhang stap
le strand for 7-7 zipper, top) ATGGCA

SEQ ID NO: 45 (fastening sequence of overhang stap
le strand for 7-7 zipper, top) TGGTTC

SEQ ID NO: 46 (fastening sequence of overhang stap
le strand for 7-7 zipper, top) ACTGCGT

SEQ ID NO: 47 (fastening sequence of overhang stap
le strand for 7-7 zipper, top) GGCCCCA

SEQ ID NO: 48 (fastening sequence of overhang stap
le strand for 7-7 zipper, bottom) CTCGAGG

SEQ ID NO: 49 (fastening sequence of overhang stap
le strand for 7-7 zipper, bottom) TTAAATT

SEQ ID NO: 50 (fastening sequence of overhang stap
le strand for 7-7 zipper, bottom) ACTCAA

SEQ ID NO: 51 (fastening sequence of overhang stap
le strand for 7-7 zipper, bottom) CCGCTC

SEQ ID NO: 52 (fastening sequence of overhang stap
le strand for 7-7 zipper, bottom) CAATAAT

SEQ ID NO: 53 (fastening sequence of overhang stap
le strand for 7-7 zipper, bottom) AGGTTTA

SEQ ID NO: 54 (fastening sequence of overhang stap
le strand for 10-10 zipper, top) TGGGTTATCT

SEQ ID NO: 55 (fastening sequence of overhang stap
le strand for 10-10 zipper, top) CAGGTTGGAC

SEQ ID NO: 56 (fastening sequence of overhang stap
le strand for 10-10 zipper, top) TCTTCAGGTT

SEQ ID NO: 57 (fastening sequence of overhang stap
le strand for 10-10 zipper, top) TGCTTTACAT

SEQ ID NO: 58 (fastening sequence of overhang stap
le strand for 10-10 zipper, bottom) GCTTATTAT
G

SEQ ID NO: 59 (fastening sequence of overhang stap
le strand for 10-10 zipper, bottom) AGCTGGTGC
T

SEQ ID NO: 60 (fastening sequence of overhang stap
le strand for 10-10 zipper, bottom) TGGTGATTC
T

SEQ ID NO: 61 (fastening sequence of overhang stap
le strand for 10-10 zipper, bottom) AAACTTTAC
T

SEQ ID NO: 62 (original latch sequence for S-gene)
GTGAAGGATTTCAACGTACACTTTGTTTCTGAGAGAGGGTCAAG
TGCACAGTCTACAGCATCTGTAATGGTTCCATTTTCATTATATTTTAATA
GAAAAG

SEQ ID NO: 63 (toehold sequence of original latch
sequence for S-gene) GGTTGA

SEQ ID NO: 64 (new latch sequence for S-gene)
GTGAAGGATTTCAACGTACACTTTGTTTCTGAGAGAGGGTCAAGTGCAC
AGTCTA CAGCATCTGTAATGGTTCCATTTTCATTATATTTTAATAGAAA
AG

SEQ ID NO: 65 (toehold sequence of new latch seque
nce for S-gene) GTGAAGGA

SEQ ID NO: 66 (latch sequence for N-gene) ATT
GGAACGCCTTGTCCTCGAGGGAATTTAAGGTCTTCCTTGCCATGTTGAGT
GAGAGCGGTGAACCAAGACGCAGTATTATTGGGTAAACCTTGGGGCC

SEQ ID NO: 67 (toehold sequence of latch sequence
for N-gene) ATTGGAAC

SEQ ID NO: 68 TTCTGAAACATGAAAGGAGGGAGGAATCAGT
AGCAAGAAAAAACAGAGTTTGCGGA

SEQ ID NO: 69 ATGAATTTATTTTGCTAAACAACTGGTCGCT
GAGGCTTGCAGGCCGCTTTTGCGGG

SEQ ID NO: 70 ATCGTCACCCTCAGCACATAACCGTAAGAAT
AAAATAAGAATAGATTGGCAAATC

SEQ ID NO: 71 CGATAGTTCGGCTACAGAGAAAACTTTTTCA
AAGATTAAGTCATTTGA

SEQ ID NO: 72 CCTTATTAAAGCCGTTCGAACCTCCCAGTAA
TACGGATTCTCAAGAGT

SEQ ID NO: 73 CGGAAATTATTTCGGAAGCGTCATACATGGC
TTAATGCGCGTCACACG

SEQ ID NO: 74 TTTCAGCGGTCGTCTTAGAGAATACCGGAAC
CTAACATCACACCGAGT

SEQ ID NO: 75 TTGATATAGTACCGTACATCTTTTGAATTAA
CACCTCAAATTAGAAGT

SEQ ID NO: 76 CGGGTATTACATAAAGTAATAAGAGCGACAG
AATCGTCTGCGAGCACG

SEQ ID NO: 77 TTACCAGCATTAGGATAACAAATAAATCCTC
ACGTTAGAAAAATACCT

SEQ ID NO: 78 CCAGTACAGCGGATAAGCAGGTCAGACGATT
GGCCGATTAGCCAGCCA

SEQ ID NO: 79 TTTGTCACTCCAAAAAGCTAACGAACCAGTA
TCAAACATCAATCATAG

SEQ ID NO: 80 AAAGTTTTGAGTGAGACCCAATCCAACACCG
GGAAACAGTCCTTGAAA

SEQ ID NO: 81 AAGACTTTGAATTTCAAATTCTTGCGTCTTT
AATTCGACAAATCTAA

SEQ ID NO: 82 CCTAATTTAAACGAAACAATCATAAGGGAAC
ACGGTGTATCGCGCAG

SEQ ID NO: 83 AGCCTTTAAAAGACACGAGATAACACTGTAG
CGCTCATGGTCAGAGC

SEQ ID NO: 84 CGGGTAAAATTGTATCAGTAGGGAATTTTAT
GAACGTTAAGTGCCAC

SEQ ID NO: 85 AGAGAAGGGCCAAAGAGCGTCAGCCACAAGA
CCTGCAACTTAATTTT

SEQ ID NO: 86 TAAAGGAACCCTCATAGACGGGACATAATCA
ACTATCGGATCCTGAG

SEQ ID NO: 87 ATAAGTCCACTCATCTTGTTACTTAGCCGGA
GACCTTCAGCCTGATT

SEQ ID NO: 88 ACTAAAACTGAACAAGTTTTCGAGCCGACTT
AAGGAGCGGAACGAAC

SEQ ID NO: 89 CCAACCTAACGAGCATACAACGCCCAAGATT
ATTATCATGTGAGGCG

SEQ ID NO: 90 TTCGAGGTTTCATGAGGATGCAAATCCAATC
TTTATCAAAAGAAAAC

SEQ ID NO: 91 AGTAACAGCTTGAGCTAGCACCAATAGCCGA
ACAAAGTTACCAGAAG

SEQ ID NO: 92 TGAAAATCAACTACAAAGTCAGAGATAGCCC
CTATTACCAAGGGATT

SEQ ID NO: 93 GAAACGCTGTCTTTCCCAGCTACCTTAATTG
TCAATTACCTCCGGCT

SEQ ID NO: 94 AGTACCAGAATCAATATTTCGGTCGGTAATT
GCAAATGAAAACTCGT

SEQ ID NO: 95 TAATCGGCATACGTAATAGGTTGGGTTATAT
ATTTTTAACCTGAGCA

SEQ ID NO: 96 AACCGATTTATTAAGAAAGCGCAGTCTCTGA
TGCTTTGAAAATGGAT

SEQ ID NO: 97 CCTGCCTATTCATTATCACCAATTACCGAAG
AAAATACCGAATTATC

SEQ ID NO: 98 GTAGCAAGCGCCGACAAAAGCCTAAAATAAA
TTGAGGATTATCAAA

SEQ ID NO: 99 GCCCACGGCGAAAGATTCATCTTCTGACCTC
TTAGAATACATAAAT

SEQ ID NO: 100 ACAGACAGTTGCGAATCCAGTTACGTTTAG
TAACAATTACGCTGAG

SEQ ID NO: 101 CCCTCATTCAGGAGGCACCCTCAGAGCCGC
CAGTGAGGCCTTGCCT

SEQ ID NO: 102 GAACAAGCCGCAGTAAGCTATCTGAAACCA
TTCACCACGCTACAG

SEQ ID NO: 103 TGTCGAAATCCGCGACATAAAGTAGTTTATCAC
ATCGTAG

SEQ ID NO: 104 CGCCACCCTCAGAACCGAACCGCCACCCTCAGG
TTAGTAA

SEQ ID NO: 105 AACAACATGTTCAGCTAATGCAGACGCGAAAAT
AAATTG

SEQ ID NO: 106 CCGGAATAGGTGTATCACCGTACTTTCAGGGA

SEQ ID NO: 107 ATACCAAGACGCGCCTCCGACAAATTCTAAGA

SEQ ID NO: 108 AGCCACCAACATAAAAGGTCAGTTAGAGCCGT

SEQ ID NO: 109 TTTGATGACGGAAACGAAGGTGAATTAAGACT

SEQ ID NO: 110 ACGCGAGGAAGAAAAGTGGCATGATTATCACC

SEQ ID NO: 111 AAACGCAATAATAACGGAATACCCTACCGCGC

SEQ ID NO: 112 CCCTTTTTCGTTTTAGTTTATTTTACAATAG

SEQ ID NO: 113 ATCCGGTAAGGTAAAGTAATTCTGTCCAGAC

SEQ ID NO: 114 ATTTAGGCCAGACGGTGAGGCAAAAGAATAC

SEQ ID NO: 115 CCAATAGGAACCCATAGTATAGCAGAGCCGC

SEQ ID NO: 116 AAATACATAAACCAACCTTAAATAACATGTA

SEQ ID NO: 117 GAATCATAAAAGAACTAAGCAGTTACCATT

SEQ ID NO: 118 CGCCAGCACGCCAGACCTTGCTG

SEQ ID NO: 119 AAAATCACCAGCATTTGGGAAT

SEQ ID NO: 120 CCGCCACCCTCTCAGAGCCGCC

SEQ ID NO: 121 TGATAAATAAGTGGTTTGAAAT

SEQ ID NO: 122 AAAATAGCAGCTTTTGTTTAAC

SEQ ID NO: 123 ATCATCGCCTGCAAAGTACAAC

SEQ ID NO: 124 ATAGAAGGCTTCCAATAGCAAG

SEQ ID NO: 125 ACATTCTGGCCA

SEQ ID NO: 126 TCAGATGATGGC

SEQ ID NO: 127 CATATTCCTGAT

SEQ ID NO: 128 TCTTGACAAGAA

SEQ ID NO: 129 GGATATTCATTA

SEQ ID NO: 130 CAGTAATAAAAG

SEQ ID NO: 131 GTCACCGATGCCCGTATGTACTGGTAATAAG

SEQ ID NO: 132 AAGAGAATCTGCTCCATTGACCCCCAGCGATT

SEQ ID NO: 133 AGCAAGGCTACAGGAGTAAACAGTTAATGCCC

SEQ ID NO: 134 AAAAAGATGTCAGAAGATTAAATTAACCAG
GGTCTGAGGATCCCCGGGTACCGA

SEQ ID NO: 135 ATAAAGAAAAATTATTGTGCCGGATTTGTT
AATTCTTTTCACCAGGGA

SEQ ID NO: 136 TAGAAAGATGCCAGAGCGCATCGTGTGAGC
GACTTTCCAGGATGGTGG

SEQ ID NO: 137 AGAGATAGATCGCCATAAACAGGAGGCCTC
TTCCAGCTGGACGGATAA

SEQ ID NO: 138 GGCTATTATGGCACAGGCCGGAGACTAGGG
CGGTAAAGCACTAAATCG

SEQ ID NO: 139 AGAATACGGTCTTTAAATTGTAAATGCGCA
ACCGATTAAGTTGGGTAA

SEQ ID NO: 140 GAATAAGGCTTGCCCTGCAGCACCGTCGGT
GGTCAATCCGCCGGGCGC

SEQ ID NO: 141 ATTTCAACTTTAATCACCGGACTTATCAAA
CTCCAGCACGATGGTCAT

SEQ ID NO: 142 TCTGGAAGTTTCATTCTTTCATTTTTTTAA
ATACCGCCTGGCGTATTG

SEQ ID NO: 143 AATGCTTTCCGGAAGCTTTCAACGACTAAT
AGGACTCCAACGTCAAAG

SEQ ID NO: 144 ATCGTCATGAGTACCTACCCTGTATCATAC
AGTTTGGAACAAGAGTCC

SEQ ID NO: 145 CAGGACGTTGGGAAGAGTCCGTTTAAAATC
CCACTCTGTCGGAAGCA

SEQ ID NO: 146 CACCACACCCGCCGCGTGGCGAGACGTTCT
AGAATGCCGGACCCCGG

SEQ ID NO: 147 CTGGCAAGTGTAGCGGGCGGGCGCAGTCAA
ATCTACAAAGTAATCGT

SEQ ID NO: 148 TACTTCTGGAATATACTTTGTGAGAGATAG
AGGCCAGTGCCAAGCTT

SEQ ID NO: 149 AACCAGAAAACAGTTGTCTGGCCACAGTAT
CAAATTGTTCACGGTCA

SEQ ID NO: 150 TTCATCAAAGAAACAAAACGTACACGGCTG
GAATCAGCGGGGTCATT

SEQ ID NO: 151 CATATCAATTGCGTAGGGATAGCGCAGCAA
CCGGGTCACTGTTGCCC

SEQ ID NO: 152 TAAAAACCCAACTAATTGCCGCCAGCACAT
CTTACACTGGTGTGTTC

SEQ ID NO: 153 AAAAGGAAACCCTCGGCACTCCAGGAACGC
CGGCGGTTTGCCCTGAG

SEQ ID NO: 154 CCATAAACGTTTTAACTCATATAGGGGCGC
GATCAGGGCGATGGCCC

SEQ ID NO: 155 CAAATCAACGTAACAACCAGCTTAGCGCCA
TGCACGGGACGAAAGGG

SEQ ID NO: 156 ACATCGGGTATAATCCGGTGCGAGATTGTA
TCATATGTAGAGGGTA

SEQ ID NO: 157 CCTAAAACAACCCTTCATTCAACAAGGAAG
GCCCCGATTTAGAGCT

SEQ ID NO: 158 CTATTATATAAGAGGGTGTAGGTGTCAATA
ACCCAAATCAAGTTTT

SEQ ID NO: 159 GCGATTTTAAGAACTCGGCCAGAGCAGTTG
GGCCGTTTTATCCGCT

SEQ ID NO: 160 AGAAGTTTTTCATCAGACATAAATTTCGTC
TCATCAGATGCCGGGT

SEQ ID NO: 161 ACTATCATATTACGAGAACAGCGGGTAGAACGT
AAAGGTT

SEQ ID NO: 162 AAATATCGTCAAAAATAACCAATAGCCAGCTTT
CGTAATC

SEQ ID NO: 163 CGCCAGGGAGAGAATCGATGAACGGGCTATCAG
GTCATTG

SEQ ID NO: 164 CCCAATTCTGCGAACGCGCAAATGAAAGATTTC
TTGAGA

SEQ ID NO: 165 GGATTAGAAAATATTCATTAAATAACCGTGCAT
ACGAGC

SEQ ID NO: 166 GTCCACGCGAATCGGCCAACGCGCCCTGTGTGG
GCCTCA

SEQ ID NO: 167 ATAAAGCTAAATCGGTTGTATCCCTTTAATTGC
G

SEQ ID NO: 168 CGTGCCTGTTCTTCGCGTAATGGGTCAGCGTG

SEQ ID NO: 169 GGTGCTGCGGCCAGAAGTTAACGGCGTCGCTG

SEQ ID NO: 170 CATGTTTTAAATATGCTCAATTCTCAAGGATA

SEQ ID NO: 171 CGGGCAACAGCTGATAACCATCACCTGTTTA

SEQ ID NO: 172 AGCAAATCTGCGGCGGGCGGTTGGTTTGAGG

SEQ ID NO: 173 TTGCGCTCAAAGCCTGGGGTGCCCGCAGTGT

SEQ ID NO: 174 GTCAGATAATAATGGATTCAGGCCGTTAATA

SEQ ID NO: 175 GGACGACGTTCCTGTGCATTAATTGGTTTG

SEQ ID NO: 176 TCTTTGCTCGTCATAGTTTCTGTAAATTTC

SEQ ID NO: 177 CTTAATTGCTGAATACCAATAAAATAC

SEQ ID NO: 178 TTTTTCCTGTTTTCGGGAAACCTGTCG

SEQ ID NO: 179 AGGCGGCCTGATTGCCTGCCGGTG

SEQ ID NO: 180 TCTCACGGGCCTCCTCACAGTACG

SEQ ID NO: 181 ACGAGTAGTAAATTGCTGGTCA

SEQ ID NO: 182 CCGTAATGGGATGGGAACAAAC

SEQ ID NO: 183 TTAAACGATGCTTTAGTGATG

SEQ ID NO: 184 GCAAAATTAAGCAATAAA

SEQ ID NO: 185 TGAGTGTTTCGGCAAAACCAAAAACCGTCG
GACGTCCAATTAGTAAAA

SEQ ID NO: 186 AGGAGCACGTGAGTGAATAACCTTATCGTC
GC

SEQ ID NO: 187 CGGAAATTATTTCGGAAGCGTCATACATGG
CTTAATGCGCGTCACACGACCAGTAATAAA

SEQ ID NO: 188 CCTGCCTATTCATTATCACCAATTACCGAA
GAAAATACCGAATTATCATCATATTCCTG

SEQ ID NO: 189 CCTTATTAAAGCCGTTCGAACCTCCCAGTA
ATACGGATTCTCAAGAGTAATCTTGACAAG

SEQ ID NO: 190 ATTCTGGCCAACAGAGATAGATCGCCATAA
ACAGGAGGCCTCTTCCAGCTGGACGGATAA

SEQ ID NO: 191 AGATGATGGCAATTCATCAAAGAAACAAAA
CGTACACGGCTGGAATCAGCGGGGTCATT

SEQ ID NO: 192 ATATTCATTACCCAAATCAACGTAACAACC
AGCTTAGCGCCATGCACGGGACGAAAGGG

SEQ ID NO: 193 CGAAAG TGCAT GGGGAA

SEQ ID NO: 194 TTCCCC ATGCA CTTTCG

SEQ ID NO: 195 AAAAGAGTCTGTCCATCACGCAAATTAACA
GTGTAC

SEQ ID NO: 196 CAGAAACCGTTGTAGCAATACTTCTTTGAA
AGGAATTGAGGAAGGTTATGACTGT

SEQ ID NO: 197 ATGGTGCTAAAATATCTTTAGGAGCACGTG
AGTGAATAACCTTGCTTCTAATGAA

SEQ ID NO: 198 ATTAAAATGTAAATCGTCGCTATTAATTAA
TTTTCCAAATTTAAGCGTTAAAATATATTCTTCAACAG

SEQ ID NO: 199 GTTGAAAGTCATTTTTGCGGATGTCCTTTT
GATAAGAGCCTCTCT

SEQ ID NO: 200 CACTTCCTGGATAGCGTCCAATTAGTAAAA
TGTTTAGAATTACA

SEQ ID NO: 201 AATGGAACTAACGGAACAACATTCGTTAAT
AAAACGAACTTTTCTGTGAAGGATTTCAACGTACACTTTGTTTCTGAGAG
AGGGTCAAGTGCACAGTCTACAGCATCTGTAATGGTTCCATTTTCATTAT
ATTTTAATAGAAAAG

SEQ ID NO: 202 CTTTTCTATTAAAATATAATGAAAATGGAA
CCATTACAGATGCTGTAGACTGTGCACTTGACCCTCTCTCAGAAACAAAG
TGTACGTTGAAATCCTTCAC

SEQ ID NO: 203 GTGAAGGATTTCAACGTACACTTTGTTTCT
GAGAGAGGGTCAAGTGCACAGTCT

SEQ ID NO: 204 ACAGCATCTGTAATGGTTCCATTTTCATTA
TATTTTAATAGAAAAGTCCTAGGT

SEQ ID NO: 205 AAAAGAGTCTGTCCATCACGCAAATTAACA
AACAAAGTG

SEQ ID NO: 206 GACCCTCTCTCGTTGTAGCAATACTTCTTT
GAAAGGAATTGAGGAAGGTTATGGAACCATTA

SEQ ID NO: 207 ATATAATGAACTAAAATATCTTTAGGAGCA
CGTGAGTGAATAACCTTGCTTC

SEQ ID NO: 208 TGTAAATCGTCGCTATTAATTAATTTTCCA
AATTTAAGCGTTAAAATATATTCTTCAACAG

SEQ ID NO: 209 TAGGTTGAAAGTCATTTTTGCGGATGTCCT
TTTGATAAGAGCTGTGCACTT

SEQ ID NO: 210 CAGATGCTGTCTGGATAGCGTCCAATTAGT
AAAATGTTTAGATTCTATTAAA

SEQ ID NO: 211 CTAACGGAACAACATTCGTTAATAAAACGA
A

SEQ ID NO: 212 AAAAGAGTCTGTCCATCACGCAAATTAACC
TCGAGG

SEQ ID NO: 213 TTAAATTCGTTGTAGCAATACTTCTTTGAA
AGGAATTGAGGAAGGTTATACTCAA

SEQ ID NO: 214 CCGCTCCTAAAATATCTTTAGGAGCACGTG
AGTGAATAACCTTGCTTCCAATAAT

SEQ ID NO: 215 AGGTTTATGTAAATCGTCGCTATTAATTAA
TTTTCCAAATTTAAGCGTTAAAATATATTCTTCAACAG

SEQ ID NO: 216 ACAAGGCGTCATTTTTGCGGATGTCCTTTT
GATAAGAGGAAGACC

SEQ ID NO: 217 ATGGCACTGGATAGCGTCCAATTAGTAAAA
TGTTTAGATGGTTC

SEQ ID NO: 218 ACTGCGTCTAACGGAACAACATTCGTTAAT
AAAACGAAGGCCCCA

SEQ ID NO: 219ATTGGAACGCCTTGTCCTCGAGGGAATTTAAGGTCT
TCCTTGCCATGTTGAGTGAGAGCGGTGAACCAAGACGCAGTATTATTGGG
TAAACCTTGGGGCC

SEQ ID NO: 220GGCCCCAAGGTTTACCCAATAATACTGCGTCTTGGT
TCACCGCTCTCACTCAACATGGCAAGGAAGACCTTAAATTCCCTCGAGGA
CAAGGCGTTCCAAT

SEQ ID NO: 221 ATTGGAACGCCTTGTCCTCGAGGGAATTTA
AGGTCTTCCTTGCCATGTTGAGTG

SEQ ID NO: 222 AGAGCGGTGAACCAAGACGCAGTATTATTG
GGTAAACCTTGGGGCCGACGTTGT

SEQ ID NO: 223 AAAAGAGTCTGTCCATCACGCAAATTAACG
CTTATTATG

SEQ ID NO: 224 AGCTGGTGCTCGTTGTAGCAATACTTCTTT
GAAAGGAATTGAGGAAGGTTATTGGTGATTCT

SEQ ID NO: 225 AAACTTTACTCTAAAATATCTTTAGGAGCA
CGTGAGTGAATAACCTTGCTTC

SEQ ID NO: 226 TGTAAATCGTCGCTATTAATTAATTTTCCA
AATTTAAGCGTTAAAATATATTCTTCAACAG

SEQ ID NO: 227 TGGGTTATCTGTCATTTTTGCGGATGTCCT
TTTGATAAGAGCAGGTTGGAC

SEQ ID NO: 228 TCTTCAGGTTCTGGATAGCGTCCAATTAGT
AAAATGTTTAGATGCTTTACAT

SEQ ID NO: 229 CTAACGGAACAACATTCGTTAATAAAACGA
A

SEQ ID NO: 230 TAAAGTGTACTGCCCGGTAACAACCATTAT
GTTAATTGCTCCTTTT

SEQ ID NO: 231 TTCTCCGTAGGTCACGTTGGTGTAGCCGCA
CACAACATTCGTTAAT

SEQ ID NO: 232 TTCCGAAAGTTCCAGGCAAGGCAAAGAATT
ATGCGGATGGCTTAGAG

SEQ ID NO: 233 TACCTGCAGCCTGTGCGTAAAAAAAGATGG
GGGGGTAAACTGCGGA

SEQ ID NO: 234 CACTGCGCGCCAGCGGGTTCCGGCAAACGC
GAAAATCTAATTACAGG

SEQ ID NO: 235 CACAATTCTGCCAGCTAGCCAGCTAGCCTT
TAAAACTCCAACAGGTCA

SEQ ID NO: 236 ACTATTAAGGCGAAAAGCGGGAGATTCATC
AACATTGAATTTTGCAAA

SEQ ID NO: 237 CACACAACATCTGCCATGTACATCGTTGAG
ATATACCAGT

SEQ ID NO: 238 CCCCAGCAAGAACGTGTAGTAGCATTAACA
TTAATGCTGTAGCTCAA

SEQ ID NO: 239 ACCACATTAAAATAGCGAGAGGCTCCCCCT
CA

SEQ ID NO: 240 GCAGCCTCGGCTCATTTTAGGAAT

SEQ ID NO: 241 AGAGTTGCAACCGTCTAGCTGAAAAGGTGG
CAAACTAAAGTCAAAGCG

SEQ ID NO: 242 GGCGAAAAGCAAGCGAAAATTTTAATTCGC
CAGAAAACACGACGA

SEQ ID NO: 243 TGCTCATTGCAGATACTACCTTAT

SEQ ID NO: 244 AGCTGTTTGGGGAGAATCAAAAATTAGAAC
CTTCGAGCTTACGGTG

SEQ ID NO: 245 GGAAGATCTTTACCAGGAGAATGA

SEQ ID NO: 246 TACCGGGGAACATCCCCTCATAACGGAACG
TGTTGTGAATATAACGCC

SEQ ID NO: 247 GCTATATCATATAACAAGACTTC

SEQ ID NO: 248 ACTACGTGTGCCCTTCGCAATGCCCATTTT
TTCAGGTCTTAGAGCAAC

SEQ ID NO: 249 GCTCGAATTCCGGCACACGCAGAGCATAGT
AATGGTTTA

SEQ ID NO: 250 GGCGCCAGGGTGGTTTATCAGCTTGAGTAA
TAAGCCCGAAGTTGATT

SEQ ID NO: 251 TGCGGCTGGTCCGTGAAAAAAGAGCGCTTC
TGTGCACGTATACCCTGA

SEQ ID NO: 252 GTGCTGGTGGCTTGAGAAACAGAA

SEQ ID NO: 253 TTTTGTTATGAGAAAGACAATATTATTGCA
TC

SEQ ID NO: 254 TTGGGGTCCCTGAGAGCAAAAGGGAAATTC
GCCAAAGCGGGAACCTAC

SEQ ID NO: 255 GCAAACATTTTCCCACAAAGCGCGTGGTGA
AGATTTTCAACACCAGA

SEQ ID NO: 256 GAGGTGCCACCATTAGATACATTTAGTAGA
TTTAGTTTG

SEQ ID NO: 257 GGTTGCGAAAACGACCTTTCTCCCATTCGC
CAAGGGTTATTTGAAT

SEQ ID NO: 258 ACGTTGTGTATGAGCCGCAAGAATGCCAAC
GGACGAGAAGGTTTAAC

SEQ ID NO: 259 GCTATTTTAAGGGAGCGAAGAAAGCGAAAG
GATCACGCTGAAAGCGTA

SEQ ID NO: 260 GAACCCTATGAGAGATCACCATCTATTTAA
TGCGCGAATTGGATTA

SEQ ID NO: 261 AAAACTAGTGCAAGGTGTTGGGATCCCGGA
AAGTAACAGCATTCAGT

SEQ ID NO: 262 GGATGTGCCATGTCAATAAGCAAAAATATG
ATTGACCTGCGCGTAAC

SEQ ID NO: 263 GCAGGCGCGGAGCCGCTTTACCAGAGGGCG
ATCTGATTGTCTGATAGC

SEQ ID NO: 264 TCAGAGGTTTTCGCACTGCCATCCCACGCA
AAGCTGCTTACCTTTT

SEQ ID NO: 265 CTTCTTTGATTAGTAAGCCTCCCAGAGCCA
CTTTAGTAC

SEQ ID NO: 266 TATTAATTAATTTTCCAAATTTAAGCGTTA
AAATATATTCTTCAACAG

SEQ ID NO: 267 AAAAGAGTCTGTCCATCACGCAAATAGCAA
TA

SEQ ID NO: 268 AACAGTTGAAAGGAATTGAGGAAGATATCT
TT

SEQ ID NO: 269 CAATATATTAACAACTAAACGATTCTTTAC
AGTCCAGACAGCCACCA

SEQ ID NO: 270 GAGTAGAACAATATCTACAGGGAATTATTT
ATATAGAAAGCAACCATC

SEQ ID NO: 271 CAATAGATTTTAATGAATCATAATAGTTAA
TCAGCATCGGAACGAGG

SEQ ID NO: 272 ACATAGCGATAGCTTATATATTTTTACTAG
AAATGACAAGAACAAC

SEQ ID NO: 273 AAGTGTTTTTATAATCACCAGAACGGAACC
AGTAGCAAGCAACGATCT

SEQ ID NO: 274 CCCTCAATGAACTCAAAAATCACCCACCAC
C

SEQ ID NO: 275 ATTACCTTTAATACATCAGCCATAGCGCAT
TAGTTAGCGT

SEQ ID NO: 276 GTAATATCCTTGCTGATGAACACCCTAATT
TGAATAATTTCTTGATAC

SEQ ID NO: 277 ATTAGACTTTACATTTATCATATGAAGAAC
GCGAGGCTTTGAGGACTA

SEQ ID NO: 278 AAGAGTCAATAGTGAAGCAAGACACGTTAT
ACTTAAACAGTTTCACGT

SEQ ID NO: 279 TTAGACAGGAACGGTATTGACAGGCGTTTG
CACACTGAGGCATTCC

SEQ ID NO: 280 AGCATCACCAGAACAACTTATTAGAGGTTG
AGGTGCCGTCGAGAGGG

SEQ ID NO: 281 AAAATTAATTACAAACCCAGAGCCTGAACA
ACGCCTGTATTTCGTCA

SEQ ID NO: 282 TTGCAACAGCCAGCAGAGCGCTAATTACCA
ACAAAGGCTAGCTTGCT

SEQ ID NO: 283 ATTAAATCTGATGAAAAAAGCCAATAAATG
CTGAAGTTTCCATTAAA

SEQ ID NO: 284 GTCTGAGAGACTACCACTATATGCGCTCAA
CGGTTTATCCCAAAAGG

SEQ ID NO: 285 GGGAGCTAAACAGGAGGCCTTGAATCGGCA
TGAAAATTCAAGTTTAT

SEQ ID NO: 286 GCTGAGAGGAAAAACGCGTTTTCTATTCAC
ATAGCGGGGTTTTGCTC

SEQ ID NO: 287 AAAGAAGACTTTGCCCCCTGAATCTATCAG
ACACGGAATATATGGT

SEQ ID NO: 288 ACATTTTTAACACCGATTGAGTTTTTGCAC
CTTATCATCCAATCAA

SEQ ID NO: 289 AAAAGTTTTATTCATTAGAATCGCCCAACT
TTTGCCACTACGAAGGCA

SEQ ID NO: 290 GAAAGAGGACAGATGACGAACTGACATATT
TAGTAGAAATCCAAGAA

SEQ ID NO: 291 TATAACGTGCTTTCCTTTAAAGCCTGCCTT
TACAAAAGGGATATAAAA

SEQ ID NO: 292 GTCAGTATGACGCTCAATCAAGTTAGAATG
GAGGCTGAGACTCCTCA

SEQ ID NO: 293 AGGCGAATGAGTAACAGTTGCTATAAGCCC
AAGTGGCAACCGACATTC

SEQ ID NO: 294 TATTTACAGAAGATACAATGAATTTTGAAG
GTACCGCATATCCCAT

SEQ ID NO: 295 ACAAAGAAATACCAAGTTACAAAACAGACC
A

SEQ ID NO: 296 GGCGCATAGGCTGGCTACGAGGCGAGAGGC
AAAAAATAACTCATCGA

SEQ ID NO: 297 GGCGCGTACTATGGTATTTACCGAGCACCG
TGAAGGTAAACGTAGA

SEQ ID NO: 298 CACCAGCATTGGCAGATCGATAGCTTCCAG
TAACCTATTA

SEQ ID NO: 299 GCTTTGAACCACCAGGCGGGAGGATAGCAA
TTGTTAGCAAATATTGA

SEQ ID NO: 300 (S-gene (New, 100 after)) CTTT
TCTATTAAAATATAATGAAAATGGAACCATTACAGATGCTGTAGACTGTG
CACTTGACCCTCTCTCAGAAACAAAGTGTACGTTGAAATCCTTCAC

SEQ ID NO: 301 (N-gene) ATCAGCGAAATGCACCCCGCA
TTACGTTTGGTGGACCCTCAGATTCAACTGGCAGTAACCAGAATGGAGAA
CGCAGTGGGGCGCGATCAAAACAACGTCG

SEQ ID NO: 302 (Sgene2 Zipper - Closure) GTGA
AGGATTTCAACGTACACTTTGTTTCTGAGAGAGGGTCAAGTGCACAGTCT
ACAGCATCTGTAATGGTTCCATTTTCATTATATTTTAATAGAAAAG

SEQ ID NO: 303, Sequence Sgene SARS-CoV-2 Zipper -
Closing Sequence GGTTGAAGATAACCCACATAATAAGCT
GCAGCACCAGCTGTCCAACCTGAAGAAGAATCACCAGGAGTCAAATAACT
TCTATGTAAAGCAAGTAAAGTTT

SEQ ID NO: 304, Sequence Ngene SARS-CoV-2 Zipper-c
losing sequence ATTGGAACGCCTTGTCCTCGAGGGAATTT
AAGGTCTTCCTTGCCATGTTGAGTGAGAGCGGTGAACCAAGACGCAGTAT
TATTGGGTAAACCTTGGGGCC

SEQ ID NO: 305, Sequence N1 Primer SARS-CoV-2 Zipp
er-closing sequence ACGTTGTTTTGATCGCGCCCCACTG
CGTTCTCCATTCTGGTTACTGCCAGTTGAATCTGAGGGTCCACCAAACGT
AATGCGGGGTGCATTTCGCTGATTT

SEQ ID NO: 306, Sequence N3 Primer SARS-CoV-2 Zipp
er-closing sequence GCGTAGAAGCCTTTTGGCAATGTTG
TTCCTTGAGGAAGTTGTAGCACGATTGCAGCATTGTTAGCAGGATTGCGG
GTGCCAATGTGATCTTTTGGTGTAT

SEQ ID NO: 307, RNAseP zipper-closing sequence
AGTCTGACCTCGCGCGGAGCCCCGTTCTCTGGGAACTCACCTCCCCGA
AGCTCAGGGAGAGCCCTGTTAGGGCCGCCTCTGGCCCTAGTCTCAGACCT
TC

SEQ ID NO: 308, FluA Matrix Zipper, closing sequen
ce ATCTTCAAGTCTCTGTGCGATCTCGGCTTTGAGGGGGCCTGA
CGGGACGATAGAGAGAACGTACGTTTCGACCTCGGTTAGAAGACTCATCT
TTCAATAT

SEQ ID NO: 309, target sequence of FIG. 25 AA
ATCAGCGAAATGCACCCCGCATTACGTTTGGTGGACCCTCAGATTCAACT
GGCAGTAACCAGAATGGAGAACGCAGTGGGGCGCGATCAAAACAACGT

SEQ ID NO: 310, TOP OH1, 5 prime of FIG. 25 G
CGATCAAAA

SEQ ID NO: 311, TOP OH2, 3 prime of FIG. 25 T
GGCAGTAAC

SEQ ID NO: 312, TOP OH3, 5 prime of FIG. 25 C
CTCAGATTC

SEQ ID NO: 313, TOP OH4, 3 prime of FIG. 25 A
GCGAAATGC

SEQ ID NO: 314, bottom OH1, 3 prime of FIG. 25
GCAGTGGGGC

SEQ ID NO: 315, bottom OH2, 5 prime of FIG. 25
CAGAATGGAG

SEQ ID NO: 316, bottom OH3, 3 prime of FIG. 25
TTTGGTGGAC

SEQ ID NO: 317, bottom OH4, 5 prime of FIG. 25
ACCCCGCATT

SEQ ID NO: 318, toehold sequence of FIG. 25 A
CGTTG

TABLE 1

Sequences

| | |
|---|---|
| SEQ ID NO | Name/Description |
| SEQ ID NO: 68 | Bottom Bar 1 |
| SEQ ID NO: 69 | Bottom Bar 2 |
| SEQ ID NO: 70 | Bottom Bar 3 |
| SEQ ID NO: 71 | Bottom Bar 4 |
| SEQ ID NO: 72 | Bottom Bar 5 |
| SEQ ID NO: 73 | Bottom Bar 6 |
| SEQ ID NO: 74 | Bottom Bar 7 |
| SEQ ID NO: 75 | Bottom Bar 8 |
| SEQ ID NO: 76 | Bottom Bar 9 |
| SEQ ID NO: 77 | Bottom Bar 10 |
| SEQ ID NO: 78 | Bottom Bar 11 |
| SEQ ID NO: 79 | Bottom Bar 12 |
| SEQ ID NO: 80 | Bottom Bar 13 |
| SEQ ID NO: 81 | Bottom Bar 14 |
| SEQ ID NO: 82 | Bottom Bar 15 |
| SEQ ID NO: 83 | Bottom Bar 16 |
| SEQ ID NO: 84 | Bottom Bar 17 |
| SEQ ID NO: 85 | Bottom Bar 18 |
| SEQ ID NO: 86 | Bottom Bar 19 |
| SEQ ID NO: 87 | Bottom Bar 20 |
| SEQ ID NO: 88 | Bottom Bar 21 |
| SEQ ID NO: 89 | Bottom Bar 22 |
| SEQ ID NO: 90 | Bottom Bar 23 |
| SEQ ID NO: 91 | Bottom Bar 24 |
| SEQ ID NO: 92 | Bottom Bar 25 |
| SEQ ID NO: 93 | Bottom Bar 26 |
| SEQ ID NO: 94 | Bottom Bar 27 |
| SEQ ID NO: 95 | Bottom Bar 28 |
| SEQ ID NO: 96 | Bottom Bar 29 |
| SEQ ID NO: 97 | Bottom Bar 30 |
| SEQ ID NO: 98 | Bottom Bar 31 |
| SEQ ID NO: 99 | Bottom Bar 32 |
| SEQ ID NO: 100 | Bottom Bar 33 |
| SEQ ID NO: 101 | Bottom Bar 34 |
| SEQ ID NO: 102 | Bottom Bar 35 |
| SEQ ID NO: 103 | Bottom Bar 36 |
| SEQ ID NO: 104 | Bottom Bar 37 |
| SEQ ID NO: 105 | Bottom Bar 38 |
| SEQ ID NO: 106 | Bottom Bar 39 |
| SEQ ID NO: 107 | Bottom Bar 40 |
| SEQ ID NO: 108 | Bottom Bar 41 |
| SEQ ID NO: 109 | Bottom Bar 42 |
| SEQ ID NO: 110 | Bottom Bar 43 |
| SEQ ID NO: 111 | Bottom Bar 44 |
| SEQ ID NO: 112 | Bottom Bar 45 |
| SEQ ID NO: 113 | Bottom Bar 46 |
| SEQ ID NO: 114 | Bottom Bar 47 |
| SEQ ID NO: 115 | Bottom Bar 48 |
| SEQ ID NO: 116 | Bottom Bar 49 |
| SEQ ID NO: 117 | Bottom Bar 50 |
| SEQ ID NO: 118 | Bottom Bar 51 |
| SEQ ID NO: 119 | Bottom Bar 52 |
| SEQ ID NO: 120 | Bottom Bar 53 |
| SEQ ID NO: 121 | Bottom Bar 54 |
| SEQ ID NO: 122 | Bottom Bar 55 |
| SEQ ID NO: 123 | Bottom Bar 56 |
| SEQ ID NO: 124 | Bottom Bar 57 |
| SEQ ID NO: 125 | Hinge connectors / Hairpin covers 1 |
| SEQ ID NO: 126 | Hinge connectors / Hairpin covers 2 |
| SEQ ID NO: 127 | Hinge connectors / Hairpin covers 3 |
| SEQ ID NO: 128 | Hinge connectors / Hairpin covers 4 |
| SEQ ID NO: 129 | Hinge connectors / Hairpin covers 5 |
| SEQ ID NO: 130 | Hinge connectors / Hairpin covers 6 |
| SEQ ID NO: 131 | No Surface Attachment OH 1 |
| SEQ ID NO: 132 | No Surface Attachment OH 2 |
| SEQ ID NO: 133 | No Surface Attachment OH 3 |
| SEQ ID NO: 134 | Top Bar 1 |
| SEQ ID NO: 135 | Top Bar 2 |
| SEQ ID NO: 136 | Top Bar 3 |
| SEQ ID NO: 137 | Top Bar 4 |
| SEQ ID NO: 138 | Top Bar 5 |
| SEQ ID NO: 139 | Top Bar 6 |
| SEQ ID NO: 140 | Top Bar 7 |
| SEQ ID NO: 141 | Top Bar 8 |
| SEQ ID NO: 142 | Top Bar 9 |
| SEQ ID NO: 143 | Top Bar 10 |
| SEQ ID NO: 144 | Top Bar 11 |
| SEQ ID NO: 145 | Top Bar 12 |
| SEQ ID NO: 146 | Top Bar 13 |
| SEQ ID NO: 147 | Top Bar 14 |
| SEQ ID NO: 148 | Top Bar 15 |
| SEQ ID NO: 149 | Top Bar 16 |
| SEQ ID NO: 150 | Top Bar 17 |
| SEQ ID NO: 151 | Top Bar 18 |
| SEQ ID NO: 152 | Top Bar 19 |
| SEQ ID NO: 153 | Top Bar 20 |
| SEQ ID NO: 154 | Top Bar 21 |
| SEQ ID NO: 155 | Top Bar 22 |
| SEQ ID NO: 156 | Top Bar 23 |
| SEQ ID NO: 157 | Top Bar 24 |
| SEQ ID NO: 158 | Top Bar 25 |
| SEQ ID NO: 159 | Top Bar 26 |
| SEQ ID NO: 160 | Top Bar 27 |
| SEQ ID NO: 161 | Top Bar 28 |
| SEQ ID NO: 162 | Top Bar 29 |
| SEQ ID NO: 163 | Top Bar 30 |
| SEQ ID NO: 164 | Top Bar 31 |
| SEQ ID NO: 165 | Top Bar 32 |
| SEQ ID NO: 166 | Top Bar 33 |
| SEQ ID NO: 167 | Top Bar 34 |
| SEQ ID NO: 168 | Top Bar 35 |
| SEQ ID NO: 169 | Top Bar 36 |
| SEQ ID NO: 170 | Top Bar 37 |
| SEQ ID NO: 171 | Top Bar 38 |
| SEQ ID NO: 172 | Top Bar 39 |
| SEQ ID NO: 173 | Top Bar 40 |
| SEQ ID NO: 174 | Top Bar 41 |
| SEQ ID NO: 175 | Top Bar 42 |
| SEQ ID NO: 176 | Top Bar 43 |
| SEQ ID NO: 177 | Top Bar 44 |
| SEQ ID NO: 178 | Top Bar 45 |
| SEQ ID NO: 179 | Top Bar 46 |
| SEQ ID NO: 180 | Top Bar 47 |
| SEQ ID NO: 181 | Top Bar 48 |
| SEQ ID NO: 182 | Top Bar 49 |
| SEQ ID NO: 183 | Top Bar 50 |
| SEQ ID NO: 184 | Top Bar 51 |
| SEQ ID NO: 185 | Top Arm Connector No OH |
| SEQ ID NO: 186 | Bottom Arm Connector No OH |
| SEQ ID NO: 187 | Anchored 12 Bott Mod 1 (Bott 6 A6) |
| SEQ ID NO: 188 | Anchored 12 Bott Mod 2 (Bott 30 C6) |
| SEQ ID NO: 189 | Anchored 12 Bott Mod 3 (Bott 5 A5) |
| SEQ ID NO: 190 | Anchored 12 Top Mod 1 (Top 4 A4) |
| SEQ ID NO: 191 | Anchored 12 Top Mod 2 (Top 17 B5) |
| SEQ ID NO: 192 | Anchored 12 Top Mod 3 (Top 22 B10) |
| SEQ ID NO: 193 | 6-6 Closing Mid Toehold 5′ |
| SEQ ID NO: 194 | 6-6 Closing Mid Toe Complement 5′ |
| SEQ ID NO: 195 | Sgene2 7-7 Bottom Zip 1 Hinge_PH |
| SEQ ID NO: 196 | Sgene2 7-7 Bottom Zip 2&3 Hinge_PH |
| SEQ ID NO: 197 | Sgene2 7-7 Bottom Zip 4&5 Hinge_PH |
| SEQ ID NO: 198 | Sgene2 7-7 Bottom Zip 6 Hinge. PH |
| SEQ ID NO: 199 | Sgene2 7-7 Top Zip 1&2 Hinge PH |
| SEQ ID NO: 200 | Sgene2 7-7 Top Zip 3&4 Hinge_PH |
| SEQ ID NO: 201 | Sgene2 7-7 Top Zip 586 Hinge_PH |
| SEQ ID NO: 202 | Sgene2 Target - Opener |
| SEQ ID NO: 203 | Sgene2 SplitZip - Closure1 |
| SEQ ID NO: 204 | Sgene2 SplitZip - Closure2 |
| SEQ ID NO: 205 | PH_10-10-10-10 SZip_Bot 1 Outside |
| SEQ ID NO: 206 | PH_10-10-10-10 SZip_Bot 2,3 Outside |
| SEQ ID NO: 207 | PH_10-10-10-10 SZip_Bot 4no5 Outside |
| SEQ ID NO: 208 | PH_10-10-10-10 SZip_Bot no6 Outside |
| SEQ ID NO: 209 | PH_10-10-10-10 SZip_Top 1,2 Outside |
| SEQ ID NO: 210 | PH_10-10-10-10 SZip_Top 1,2 Outside |
| SEQ ID NO: 211 | PH_10-10-10-10 SZip_Top 1,2 Outside |
| SEQ ID NO: 212 | Ngene 7-7 Bottom Zip 1 Hinge_PH |
| SEQ ID NO: 213 | Ngene 7-7 Bottom Zip 2&3 Hinge_PH |
| SEQ ID NO: 214 | Ngene 7-7 Bottom Zip 4&5 Hinge_PH |
| SEQ ID NO: 215 | Ngene 7-7 Bottom Zip 6 Hinge_PH |
| SEQ ID NO: 216 | Ngene 7-7 Top Zip 1&2 Hinge_PH |
| SEQ ID NO: 217 | Ngene 7-7 Top Zip 3&4 Hinge PH |
| SEQ ID NO: 218 | Ngene 7-7 Top Zip 5&6 Hinge_PH |
| SEQ ID NO: 219 | Ngene Zipper - Closure |
| SEQ ID NO: 220 | Ngene Target - Opener |
| SEQ ID NO: 221 | Ngene SplitZip - Closure1 |
| SEQ ID NO: 222 | Ngene SplitZip - Closure2 |
| SEQ ID NO: 223 | PH_10-10-10-10 NZip_Bot 1 Outside |
| SEQ ID NO: 224 | PH_10-10-10-10 NZip_Bot 2,3 Outside |
| SEQ ID NO: 225 | PH_10-10-10-10 NZip_Bot 4no5 Outside |
| SEQ ID NO: 226 | PH_10-10-10-10 NZip_Bot no6 Outside |
| SEQ ID NO: 227 | PH_10-10-10-10 NZip_Top 1,2 Outside |
| SEQ ID NO: 228 | PH_10-10-10-10 NZip_Top 1,2 Outside |
| SEQ ID NO: 229 | PH_10-10-10-10 SZip_Top 1,2 Outside |
| SEQ ID NO: 230 | BHQ Arm (Top Bar) Replacement 1 |
| SEQ ID NO: 231 | BHQ Arm (Top Bar) Replacement 2 |
| SEQ ID NO: 232 | BHQ Arm (Top Bar) Replacement 3 |
| SEQ ID NO: 233 | BHQ Arm (Top Bar) Replacement 4 |
| SEQ ID NO: 234 | BHQ Arm (Top Bar) Replacement 5 |
| SEQ ID NO: 235 | BHQ Arm (Top Bar) Replacement 6 |
| SEQ ID NO: 236 | BHQ Arm (Top Bar) Replacement 7 |
| SEQ ID NO: 237 | BHQ Arm (Top Bar) Replacement 8 |
| SEQ ID NO: 238 | BHQ Arm (Top Bar) Replacement 9 |
| SEQ ID NO: 239 | BHQ Arm (Top Bar) Replacement 10 |
| SEQ ID NO: 240 | BHQ Arm (Top Bar) Replacement 11 |
| SEQ ID NO: 241 | BHQ Arm (Top Bar) Replacement 12 |
| SEQ ID NO: 242 | BHQ Arm (Top Bar) Replacement 13 |
| SEQ ID NO: 243 | BHQ Arm (Top Bar) Replacement 14 |
| SEQ ID NO: 244 | BHQ Arm (Top Bar) Replacement 15 |
| SEQ ID NO: 245 | BHQ Arm (Top Bar) Replacement 16 |
| SEQ ID NO: 246 | BHQ Arm (Top Bar) Replacement 17 |
| SEQ ID NO: 247 | BHQ Arm (Top Bar) Replacement 18 |
| SEQ ID NO: 248 | BHQ Arm (Top Bar) Replacement 19 |
| SEQ ID NO: 249 | BHQ Arm (Top Bar) Replacement 20 |
| SEQ ID NO: 250 | BHQ Arm (Top Bar) Replacement 21 |
| SEQ ID NO: 251 | BHQ Arm (Top Bar) Replacement 22 |
| SEQ ID NO: 252 | BHQ Arm (Top Bar) Replacement 23 |
| SEQ ID NO: 253 | BHQ Arm (Top Bar) Replacement 24 |
| SEQ ID NO: 254 | BHQ Arm (Top Bar) Replacement 25 |
| SEQ ID NO: 255 | BHQ Arm (Top Bar) Replacement 26 |
| SEQ ID NO: 256 | BHQ Arm (Top Bar) Replacement 27 |
| SEQ ID NO: 257 | BHQ Arm (Top Bar) Replacement 28 |
| SEQ ID NO: 258 | BHQ Arm (Top Bar) Replacement 29 |
| SEQ ID NO: 259 | BHQ Arm (Top Bar) Replacement 30 |
| SEQ ID NO: 260 | BHQ Arm (Top Bar) Replacement 31 |
| SEQ ID NO: 261 | BHQ Arm (Top Bar) Replacement 32 |
| SEQ ID NO: 262 | BHQ Arm (Top Bar) Replacement 33 |
| SEQ ID NO: 263 | BHQ Arm (Top Bar) Replacement 34 |
| SEQ ID NO: 264 | BHQ Arm (Top Bar) Replacement 35 |
| SEQ ID NO: 265 | FAM Arm (Bott Bar) Replacement 1 |
| SEQ ID NO: 266 | FAM Arm (Bott Bar) Replacement 2 |
| SEQ ID NO: 267 | FAM Arm (Bott Bar) Replacement 3 |
| SEQ ID NO: 268 | FAM Arm (Bott Bar) Replacement 4 |
| SEQ ID NO: 269 | FAM Arm (Bott Bar) Replacement 5 |
| SEQ ID NO: 270 | FAM Arm (Bott Bar) Replacement 6 |
| SEQ ID NO: 271 | FAM Arm (Bott Bar) Replacement 7 |
| SEQ ID NO: 272 | FAM Arm (Bott Bar) Replacement 8 |
| SEQ ID NO: 273 | FAM Arm (Bott Bar) Replacement 9 |
| SEQ ID NO: 274 | FAM Arm (Bott Bar) Replacement 10 |
| SEQ ID NO: 275 | FAM Arm (Bott Bar) Replacement 11 |
| SEQ ID NO: 276 | FAM Arm (Bott Bar) Replacement 12 |
| SEQ ID NO: 277 | FAM Arm (Bott Bar) Replacement 13 |
| SEQ ID NO: 278 | FAM Arm (Bott Bar) Replacement 14 |
| SEQ ID NO: 279 | FAM Arm (Bott Bar) Replacement 15 |
| SEQ ID NO: 280 | FAM Arm (Bott Bar) Replacement 16 |
| SEQ ID NO: 281 | FAM Arm (Bott Bar) Replacement 17 |
| SEQ ID NO: 282 | FAM Arm (Bott Bar) Replacement 18 |
| SEQ ID NO: 283 | FAM Arm (Bott Bar) Replacement 19 |
| SEQ ID NO: 284 | FAM Arm (Bott Bar) Replacement 20 |
| SEQ ID NO: 285 | FAM Arm (Bott Bar) Replacement 21 |
| SEQ ID NO: 286 | FAM Arm (Bott Bar) Replacement 22 |
| SEQ ID NO: 287 | FAM Arm (Bott Bar) Replacement 23 |
| SEQ ID NO: 288 | FAM Arm (Bott Bar) Replacement 24 |
| SEQ ID NO: 289 | FAM Arm (Bott Bar) Replacement 25 |
| SEQ ID NO: 290 | FAM Arm (Bott Bar) Replacement 26 |
| SEQ ID NO: 291 | FAM Arm (Bott Bar) Replacement 27 |
| SEQ ID NO: 292 | FAM Arm (Bott Bar) Replacement 28 |
| SEQ ID NO: 293 | FAM Arm (Bott Bar) Replacement 29 |
| SEQ ID NO: 294 | FAM Arm (Bott Bar) Replacement 30 |
| SEQ ID NO: 295 | FAM Arm (Bott Bar) Replacement 31 |
| SEQ ID NO: 296 | FAM Arm (Bott Bar) Replacement 32 |
| SEQ ID NO: 297 | FAM Arm (Bott Bar) Replacement 33 |
| SEQ ID NO: 298 | FAM Arm (Bott Bar) Replacement 34 |
| SEQ ID NO: 299 | FAM Arm (Bott Bar) Replacement 35 |

What is claimed is:

1. A biosensor comprising:
a DNA origami hinge that comprises one or more scaffold strands, one or more staple strands, and one or more overhang staple strands; and
a latch strand that is capable of hybridizing to the one or more overhang staple strands and a target nucleic acid, and wherein the latch strand comprises a toehold domain.

2. The biosensor of claim 1, wherein each of the one or more overhang staple strands comprises one or more fastening sequences.

3. The biosensor of claim 1, wherein the DNA origami hinge is in a closed configuration when the latch strand is hybridized to the one or more fastening sequences.

4. The biosensor of claim 1,wherein the latch strand comprises at least 3 nucleotides complementary to each of the one or more fastening sequences.

5. The biosensor of claim 1, wherein the toehold domain does not hybridize to the fastening sequences.

6. The biosensor of claim 1, wherein the latch strand has a higher binding affinity to the target nucleic acid than to the one or more fastening sequences.

7. The biosensor of claim 1, wherein the toehold domain comprises a sequence complementary to the target nucleic acid.

8. The biosensor of claim 1,wherein the target nucleic acid displaces the one or more fastening sequences when hybridizing to the latch strand.

9. The biosensor of claim 1,wherein the DNA origami hinge is in an open configuration when the latch strand is not hybridized to the fastening sequences.

10. The biosensor of claim 1, wherein the latch strand comprises a sequence at least 80% identical to SEQ ID NO: 62, 64, 66, 303, 304, 305, 306, 307, or 308.

11. The biosensor of claim 1, wherein the toehold domain comprises a sequence at least 80% identical to SEQ ID NO: 63, 65, 67, or 318.

12. The biosensor of claim 1, wherein the fastening sequence comprises at least 80% identical to SEQ ID NOs: 2-61 or 310-317.

13. The biosensor of claim 1, wherein the target nucleic acid is a single stranded nucleic acid.

14. The biosensor of claim 1, wherein the target nucleic acid is a viral RNA.

15. (canceled)

16. The biosensor of claim 1, wherein the DNA origami hinge comprises two arms, wherein each of the two arms comprises a multi-layer structure.

17. The biosensor of claim 1, wherein the DNA origami hinge further comprises a moiety bound to one or more staple strands.

18. The biosensor of claim 17, wherein the moiety comprises BHQ, FAM, BHQ2, BHQ3, AlexaFluor 488, AlexaFluor 555, AlexaFluor 647, Cy3, Cy5, quantum dots in the equivalent fluorophore wavelengths, Iowa Black RQ, Iowa Black FQ, gold nanoparticles, biotinylated oligonucleotide/Horse Radish Peroxidase (HRP)-streptavidin, or glucose oxidase-GOx.

19. The biosensor of claim 1, wherein a first arm of the DNA origami hinge comprises one or more quenchers, and wherein a second arm of the DNA origami hinge comprises one or more fluorophores.

20. The biosensor of claim 19, wherein the first arm of the DNA origami hinge comprises at least 2 quenchers and the second arm of the DNA origami hinge comprises at least 2 fluorophores.

21. The biosensor of claim 19, wherein the one or more fluorophores and the one or more quenchers are positioned on an inner surface of the DNA origami hinge when the DNA origami hinge is in a closed configuration.

22. The biosensor of claim 19, wherein an increase in fluorescence emission is detected when the DNA origami hinge is in the open configuration as compared to the fluorescence emission detected when the DNA origami hinge is in the closed configuration.

23. A method of detecting a virus in a subject, comprising
a) obtaining a biological sample from the subject; and
b) detecting a nucleic acid of the virus using the biosensor of claim 1 .

24-30. (canceled)

31. A biosensor comprising:
a DNA origami hinge that comprises one or more scaffold strands, one or more staple strands, and one or more overhang staple strands; and
a latch strand that is capable of hybridizing to the one or more overhang staple strands and a target nucleic acid, and wherein the latch strand comprises a toehold domain,
wherein the DNA origami hinger comprises a first arm and a second arm, wherein the first arm comprises one or more quenchers, and wherein the second arm comprises one or more fluorophores.


SEQUENCE LISTING (318 sequences)

SEQ ID NO: 1 - DNA, 8064 bp, Artificial Sequence
  Synthetic construct
tgatagacggtttttcgccctttgacgttggagtccacgttctttaatagtggactcttgttccaaactggaacaacactcaaccctatctcgggctattcttttgatttataagggattttgccgatttcggaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgaattcgagctcggtacccggggatcctcaactgtgaggaggctcacggacgcgaagaacaggcacgcgtgctggcagaaacccccggtatgaccgtgaaaacggcccgccgcattctggccgcagcaccacagagtgcacaggcgcgcagtgacactgcgctggatcgtctgatgcagggggcaccggcaccgctggctgcaggtaacccggcatctgatgccgttaacgatttgctgaacacaccagtgtaagggatgtttatgacgagcaaagaaacctttacccattaccagccgcagggcaacagtgacccggctcataccgcaaccgcgcccggcggattgagtgcgaaagcgcctgcaatgaccccgctgatgctggacacctccagccgtaagctggttgcgtgggatggcaccaccgacggtgctgccgttggcattcttgcggttgctgctgaccagaccagcaccacgctgacgttctacaagtccggcacgttccgttatgaggatgtgctctggccggaggctgccagcgacgagacgaaaaaacggaccgcgtttgccggaacggcaatcagcatcgtttaactttacccttcatcactaaaggccgcctgtgcggctttttttacgggatttttttatgtcgatgtacacaaccgcccaactgctggcggcaaatgagcagaaatttaagtttgatccgctgtttctgcgtctctttttccgtgagagctatcccttcaccacggagaaagtctatctctcacaaattccgggactggtaaacatggcgctgtacgtttcgccgattgtttccggtgaggttatccgttcccgtggcggctccacctctgaaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgaggccgatactgtcgtcgtcccctcaaactggcagatgcacggttacgatgcgcccatctacaccaacgtgacctatcccattacggtcaatccgccgtttgttcccacggagaatccgacgggttgttactcgctcacatttaatgttgatgaaagctggctacaggaaggccagacgcgaattatttttgatggcgttcctattggttaaaaaatgagctgatttaacaaaaatttaatgcgaattttaacaaaatattaacgtttacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagatctctcaaaaatagctaccctctccggcattaatttatcagctagaacggttgaatatcatattgatggtgatttgactgtctccggcctttctcacccttttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttaatgctactactattagtagaattgatgccaccttttcagctcgcgccccaaatgaaaatatagctaaacaggttattgaccatttgcgaaatgtatctaatggtcaaactaaatctactcgttcgcagaattgggaatcaactgttatatggaatgaaacttccagacaccgtactttagttgcatatttaaaacatgttgagctacagcattatattcagcaattaagctctaagccatccgcaaaaatgacctcttatcaaaaggagcaattaaaggtactctctaatcctgacctgttggagtttgcttccggtctggttcgctttgaagctcgaattaaaacgcgatatttgaagtctttcgggcttcctcttaatctttttgatgcaatccgctttgcttctgactataatagtcagggtaaagacctgatttttgatttatggtcattctcgttttctgaactgtttaaagcatttgagggggattcaatgaatatttatgacgattccgcagtattggacgctatccagtctaaacattttactattaccccctctggcaaaacttcttttgcaaaagcctctcgctattttggtttttatcgtcgtctggtaaacgagggttatgatagtgttgctcttactatgcctcgtaattccttttggcgttatgtatctgcattagttgaatgtggtattcctaaatctcaactgatgaatctttctacctgtaataatgttgttccgttagttcgttttattaacgtagatttttcttcccaacgtcctgactggtataatgagccagttcttaaaatcgcataaggtaattcacaatgattaaagttgaaattaaaccatctcaagcccaatttactactcgttctggtgtttctcgtcagggcaagccttattcactgaatgagcagctttgttacgttgatttgggtaatgaatatccggttcttgtcaagattactcttgatgaaggtcagccagcctatgcgcctggtctgtacaccgttcatctgtcctctttcaaagttggtcagttcggttcccttatgattgaccgtctgcgcctcgttccggctaagtaacatggagcaggtcgcggatttcgacacaatttatcaggcgatgatacaaatctccgttgtactttgtttcgcgcttggtataatcgctgggggtcaaagatgagtgttttagtgtattcttttgcctctttcgttttaggttggtgccttcgtagtggcattacgtattttacccgtttaatggaaacttcctcatgaaaaagtctttagtcctcaaagcctctgtagccgttgctaccctcgttccgatgctgtctttcgctgctgagggtgacgatcccgcaaaagcggcctttaactccctgcaagcctcagcgaccgaatatatcggttatgcgtgggcgatggttgttgtcattgtcggcgcaactatcggtatcaagctgtttaagaaattcacctcgaaagcaagctgataaaccgatacaattaaaggctccttttggagccttttttttggagattttcaacgtgaaaaaattattattcgcaattcctttagttgttcctttctattctcactccgctgaaactgttgaaagttgtttagcaaaatcccatacagaaaattcatttactaacgtctggaaagacgacaaaactttagatcgttacgctaactatgagggctgtctgtggaatgctacaggcgttgtagtttgtactggtgacgaaactcagtgttacggtacatgggttcctattgggcttgctatccctgaaaatgagggtggtggctctgagggtggcggttctgagggtggcggttctgagggtggcggtactaaacctcctgagtacggtgatacacctattccgggctatacttatatcaaccctctcgacggcacttatccgcctggtactgagcaaaaccccgctaatcctaatccttctcttgaggagtctcagcctcttaatactttcatgtttcagaataataggttccgaaataggcagggggcattaactgtttatacgggcactgttactcaaggcactgaccccgttaaaacttattaccagtacactcctgtatcatcaaaagccatgtatgacgcttactggaacggtaaattcagagactgcgctttccattctggctttaatgaggatttatttgtttgtgaatatcaaggccaatcgtctgacctgcctcaacctcctgtcaatgctggcggcggctctggtggtggttctggtggcggctctgagggtggtggctctgagggtggcggttctgagggtggcggctctgagggaggcggttccggtggtggctctggttccggtgattttgattatgaaaagatggcaaacgctaataagggggctatgaccgaaaatgccgatgaaaacgcgctacagtctgacgctaaaggcaaacttgattctgtcgctactgattacggtgctgctatcgatggtttcattggtgacgtttccggccttgctaatggtaatggtgctactggtgattttgctggctctaattcccaaatggctcaagtcggtgacggtgataattcacctttaatgaataatttccgtcaatatttaccttccctccctcaatcggttgaatgtcgcccttttgtctttggcgctggtaaaccatatgaattttctattgattgtgacaaaataaacttattccgtggtgtctttgcgtttcttttatatgttgccacctttatgtatgtattttctacgtttgctaacatactgcgtaataaggagtcttaatcatgccagttcttttgggtattccgttattattgcgtttcctcggtttccttctggtaactttgttcggctatctgcttacttttcttaaaaagggcttcggtaagatagctattgctatttcattgtttcttgctcttattattgggcttaactcaattcttgtgggttatctctctgatattagcgctcaattaccctctgactttgttcagggtgttcagttaattctcccgtctaatgcgcttccctgtttttatgttattctctctgtaaaggctgctattttcatttttgacgttaaacaaaaaatcgtttcttatttggattgggataaataatatggctgtttattttgtaactggcaaattaggctctggaaagacgctcgttagcgttggtaagattcaggataaaattgtagctgggtgcaaaatagcaactaatcttgatttaaggcttcaaaacctcccgcaagtcgggaggttcgctaaaacgcctcgcgttcttagaataccggataagccttctatatctgatttgcttgctattgggcgcggtaatgattcctacgatgaaaataaaaacggcttgcttgttctcgatgagtgcggtacttggtttaatacccgttcttggaatgataaggaaagacagccgattattgattggtttctacatgctcgtaaattaggatgggatattatttttcttgttcaggacttatctattgttgataaacaggcgcgttctgcattagctgaacatgttgtttattgtcgtcgtctggacagaattactttaccttttgtcggtactttatattctcttattactggctcgaaaatgcctctgcctaaattacatgttggcgttgttaaatatggcgattctcaattaagccctactgttgagcgttggctttatactggtaagaatttgtataacgcatatgatactaaacaggctttttctagtaattatgattccggtgtttattcttatttaacgccttatttatcacacggtcggtatttcaaaccattaaatttaggtcagaagatgaaattaactaaaatatatttgaaaaagttttctcgcgttctttgtcttgcgattggatttgcatcagcatttacatatagttatataacccaacctaagccggaggttaaaaaggtagtctctcagacctatgattttgataaattcactattgactcttctcagcgtcttaatctaagctatcgctatgttttcaaggattctaagggaaaattaattaatagcgacgatttacagaagcaaggttattcactcacatatattgatttatgtactgtttccattaaaaaaggtaattcaaatgaaattgttaaatgtaattaattttgttttcttgatgtttgtttcatcatcttcttttgctcaggtaattgaaatgaataattcgcctctgcgcgattttgtaacttggtattcaaagcaatcaggcgaatccgttattgtttctcccgatgtaaaaggtactgttactgtatattcatctgacgttaaacctgaaaatctacgcaatttctttatttctgttttacgtgcaaataattttgatatggtaggttctaacccttccattattcagaagtataatccaaacaatcaggattatattgatgaattgccatcatctgataatcaggaatatgatgataattccgctccttctggtggtttctttgttccgcaaaatgataatgttactcaaacttttaaaattaataacgttcgggcaaaggatttaatacgagttgtcgaattgtttgtaaagtctaatacttctaaatcctcaaatgtattatctattgacggctctaatctattagttgttagtgctcctaaagatattttagataaccttcctcaattcctttcaactgttgatttgccaactgaccagatattgattgagggtttgatatttgaggttcagcaaggtgatgctttagatttttcatttgctgctggctctcagcgtggcactgttgcaggcggtgttaatactgaccgcctcacctctgttttatcttctgctggtggttcgttcggtatttttaatggcgatgttttagggctatcagttcgcgcattaaagactaatagccattcaaaaatattgtctgtgccacgtattcttacgctttcaggtcagaagggttctatctctgttggccagaatgtcccttttattactggtcgtgtgactggtgaatctgccaatgtaaataatccatttcagacgattgagcgtcaaaatgtaggtatttccatgagcgtttttcctgttgcaatggctggcggtaatattgttctggatattaccagcaaggccgatagtttgagttcttctactcaggcaagtgatgttattactaatcaaagaagtattgctacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccc

SEQ ID NO: 2 - DNA, 7 bp, Artificial Sequence
  Synthetic construct
gttatct

SEQ ID NO: 3 - DNA, 7 bp, Artificial Sequence
  Synthetic construct
tggtgct

SEQ ID NO: 4 - DNA, 7 bp, Artificial Sequence
  Synthetic construct
ttggaca

SEQ ID NO: 5 - DNA, 7 bp, Artificial Sequence
  Synthetic construct
actcctg

SEQ ID NO: 6 - DNA, 7 bp, Artificial Sequence
  Synthetic construct
agttatt

SEQ ID NO: 7 - DNA, 7 bp, Artificial Sequence
  Synthetic construct
aaacttt

SEQ ID NO: 8 - DNA, 7 bp, Artificial Sequence
  Synthetic construct
tatgtgg

SEQ ID NO: 9 - DNA, 7 bp, Artificial Sequence
  Synthetic construct
gcagctt

SEQ ID NO: 10 - DNA, 7 bp, Artificial Sequence
  Synthetic construct
cttcagg

SEQ ID NO: 11 - DNA, 7 bp, Artificial Sequence
  Synthetic construct
gtgattc

SEQ ID NO: 12 - DNA, 7 bp, Artificial Sequence
  Synthetic construct
acataga

SEQ ID NO: 13 - DNA, 7 bp, Artificial Sequence
  Synthetic construct
acttgct

SEQ ID NO: 14 - DNA, 10 bp, Artificial Sequence
  Synthetic construct
tgggttatct

SEQ ID NO: 15 - DNA, 10 bp, Artificial Sequence
  Synthetic construct
caggttggac

SEQ ID NO: 16 - DNA, 10 bp, Artificial Sequence
  Synthetic construct
tcttcaggtt

SEQ ID NO: 17 - DNA, 10 bp, Artificial Sequence
  Synthetic construct
tgctttacat

SEQ ID NO: 18 - DNA, 10 bp, Artificial Sequence
  Synthetic construct
gcttattatg

SEQ ID NO: 19 - DNA, 10 bp, Artificial Sequence
  Synthetic construct
agctggtgct

SEQ ID NO: 20 - DNA, 10 bp, Artificial Sequence
  Synthetic construct
tggtgattct

SEQ ID NO: 21 - DNA, 10 bp, Artificial Sequence
  Synthetic construct
aaactttact

SEQ ID NO: 22 - DNA, 7 bp, Artificial Sequence
  Synthetic construct
gttgaaa

SEQ ID NO: 23 - DNA, 7 bp, Artificial Sequence
  Synthetic construct
cctctct

SEQ ID NO: 24 - DNA, 6 bp, Artificial Sequence
  Synthetic construct
cacttc

SEQ ID NO: 25 - DNA, 6 bp, Artificial Sequence
  Synthetic construct
attaca

SEQ ID NO: 26 - DNA, 7 bp, Artificial Sequence
  Synthetic construct
aatggaa

SEQ ID NO: 27 - DNA, 7 bp, Artificial Sequence
  Synthetic construct
cttttct

SEQ ID NO: 28 - DNA, 7 bp, Artificial Sequence
  Synthetic construct
agtgtac

SEQ ID NO: 29 - DNA, 7 bp, Artificial Sequence
  Synthetic construct
cagaaac

SEQ ID NO: 30 - DNA, 6 bp, Artificial Sequence
  Synthetic construct
gactgt

SEQ ID NO: 31 - DNA, 6 bp, Artificial Sequence
  Synthetic construct
atggtg

SEQ ID NO: 32 - DNA, 7 bp, Artificial Sequence
  Synthetic construct
taatgaa

SEQ ID NO: 33 - DNA, 7 bp, Artificial Sequence
  Synthetic construct
attaaaa

SEQ ID NO: 34 - DNA, 10 bp, Artificial Sequence
  Synthetic construct
taggttgaaa

SEQ ID NO: 35 - DNA, 10 bp, Artificial Sequence
  Synthetic construct
ctgtgcactt

SEQ ID NO: 36 - DNA, 10 bp, Artificial Sequence
  Synthetic construct
cagatgctgt

SEQ ID NO: 37 - DNA, 10 bp, Artificial Sequence
  Synthetic construct
ttctattaaa

SEQ ID NO: 38 - DNA, 10 bp, Artificial Sequence
  Synthetic construct
aaacaaagtg

SEQ ID NO: 39 - DNA, 10 bp, Artificial Sequence
  Synthetic construct
gaccctctct

SEQ ID NO: 40 - DNA, 10 bp, Artificial Sequence
  Synthetic construct
ggaaccatta

SEQ ID NO: 41 - DNA, 10 bp, Artificial Sequence
  Synthetic construct
atataatgaa

SEQ ID NO: 42 - DNA, 7 bp, Artificial Sequence
  Synthetic construct
acaaggc

SEQ ID NO: 43 - DNA, 7 bp, Artificial Sequence
  Synthetic construct
gaagacc

SEQ ID NO: 44 - DNA, 6 bp, Artificial Sequence
  Synthetic construct
atggca

SEQ ID NO: 45 - DNA, 6 bp, Artificial Sequence
  Synthetic construct
tggttc

SEQ ID NO: 46 - DNA, 7 bp, Artificial Sequence
  Synthetic construct
actgcgt

SEQ ID NO: 47 - DNA, 7 bp, Artificial Sequence
  Synthetic construct
ggcccca

SEQ ID NO: 48 - DNA, 7 bp, Artificial Sequence
  Synthetic construct
ctcgagg

SEQ ID NO: 49 - DNA, 7 bp, Artificial Sequence
  Synthetic construct
ttaaatt

SEQ ID NO: 50 - DNA, 6 bp, Artificial Sequence
  Synthetic construct
actcaa

SEQ ID NO: 51 - DNA, 6 bp, Artificial Sequence
  Synthetic construct
ccgctc

SEQ ID NO: 52 - DNA, 7 bp, Artificial Sequence
  Synthetic construct
caataat

SEQ ID NO: 53 - DNA, 7 bp, Artificial Sequence
  Synthetic construct
aggttta

SEQ ID NO: 54 - DNA, 10 bp, Artificial Sequence
  Synthetic construct
tgggttatct

SEQ ID NO: 55 - DNA, 10 bp, Artificial Sequence
  Synthetic construct
caggttggac

SEQ ID NO: 56 - DNA, 10 bp, Artificial Sequence
  Synthetic construct
tcttcaggtt

SEQ ID NO: 57 - DNA, 10 bp, Artificial Sequence
  Synthetic construct
tgctttacat

SEQ ID NO: 58 - DNA, 10 bp, Artificial Sequence
  Synthetic construct
gcttattatg

SEQ ID NO: 59 - DNA, 10 bp, Artificial Sequence
  Synthetic construct
agctggtgct

SEQ ID NO: 60 - DNA, 10 bp, Artificial Sequence
  Synthetic construct
tggtgattct

SEQ ID NO: 61 - DNA, 10 bp, Artificial Sequence
  Synthetic construct
aaactttact

SEQ ID NO: 62 - DNA, 100 bp, Artificial Sequence
  Synthetic construct
gtgaaggatttcaacgtacactttgtttctgagagagggtcaagtgcacagtctacagcatctgtaatggttccattttcattatattttaatagaaaag

SEQ ID NO: 63 - DNA, 6 bp, Artificial Sequence
  Synthetic construct
ggttga

SEQ ID NO: 64 - DNA, 100 bp, Artificial Sequence; identical to SEQ ID NO: 62
  Synthetic construct

SEQ ID NO: 65 - DNA, 8 bp, Artificial Sequence
  Synthetic construct
gtgaagga

SEQ ID NO: 66 - DNA, 100 bp, Artificial Sequence
  Synthetic construct
attggaacgccttgtcctcgagggaatttaaggtcttccttgccatgttgagtgagagcggtgaaccaagacgcagtattattgggtaaaccttggggcc

SEQ ID NO: 67 - DNA, 8 bp, Artificial Sequence
  Synthetic construct
attggaac

SEQ ID NO: 68 - DNA, 56 bp, Artificial Sequence
  Synthetic construct
ttctgaaacatgaaaggagggaggaatcagtagcaagaaaaaacagagtttgcgga

SEQ ID NO: 69 - DNA, 56 bp, Artificial Sequence
  Synthetic construct
atgaatttattttgctaaacaactggtcgctgaggcttgcaggccgcttttgcggg

SEQ ID NO: 70 - DNA, 55 bp, Artificial Sequence
  Synthetic construct
atcgtcaccctcagcacataaccgtaagaataaaataagaatagattggcaaatc

SEQ ID NO: 71 - DNA, 48 bp, Artificial Sequence
  Synthetic construct
cgatagttcggctacagagaaaactttttcaaagattaagtcatttga

SEQ ID NO: 72 - DNA, 48 bp, Artificial Sequence
  Synthetic construct
ccttattaaagccgttcgaacctcccagtaatacggattctcaagagt

SEQ ID NO: 73 - DNA, 48 bp, Artificial Sequence
  Synthetic construct
cggaaattatttcggaagcgtcatacatggcttaatgcgcgtcacacg

SEQ ID NO: 74 - DNA, 48 bp, Artificial Sequence
  Synthetic construct
tttcagcggtcgtcttagagaataccggaacctaacatcacaccgagt

SEQ ID NO: 75 - DNA, 48 bp, Artificial Sequence
  Synthetic construct
ttgatatagtaccgtacatcttttgaattaacacctcaaattagaagt

SEQ ID NO: 76 - DNA, 48 bp, Artificial Sequence
  Synthetic construct
cgggtattacataaagtaataagagcgacagaatcgtctgcgagcacg

SEQ ID NO: 77 - DNA, 48 bp, Artificial Sequence
  Synthetic construct
ttaccagcattaggataacaaataaatcctcacgttagaaaaatacct

SEQ ID NO: 78 - DNA, 48 bp, Artificial Sequence
  Synthetic construct
ccagtacagcggataagcaggtcagacgattggccgattagccagcca

SEQ ID NO: 79 - DNA, 48 bp, Artificial Sequence
  Synthetic construct
tttgtcactccaaaaagctaacgaaccagtatcaaacatcaatcatag

SEQ ID NO: 80 - DNA, 48 bp, Artificial Sequence
  Synthetic construct
aaagttttgagtgagacccaatccaacaccgggaaacagtccttgaaa

SEQ ID NO: 81 - DNA, 47 bp, Artificial Sequence
  Synthetic construct
aagactttgaatttcaaattcttgcgtctttaattcgacaaatctaa

SEQ ID NO: 82 - DNA, 47 bp, Artificial Sequence
  Synthetic construct
cctaatttaaacgaaacaatcataagggaacacggtgtatcgcgcag

SEQ ID NO: 83 - DNA, 47 bp, Artificial Sequence
  Synthetic construct
agcctttaaaagacacgagataacactgtagcgctcatggtcagagc

SEQ ID NO: 84 - DNA, 47 bp, Artificial Sequence
  Synthetic construct
cgggtaaaattgtatcagtagggaattttatgaacgttaagtgccac

SEQ ID NO: 85 - DNA, 47 bp, Artificial Sequence
  Synthetic construct
agagaagggccaaagagcgtcagccacaagacctgcaacttaatttt

SEQ ID NO: 86 - DNA, 47 bp, Artificial Sequence
  Synthetic construct
taaaggaaccctcatagacgggacataatcaactatcggatcctgag

SEQ ID NO: 87 - DNA, 47 bp, Artificial Sequence
  Synthetic construct
ataagtccactcatcttgttacttagccggagaccttcagcctgatt

SEQ ID NO: 88 - DNA, 47 bp, Artificial Sequence
  Synthetic construct
actaaaactgaacaagttttcgagccgacttaaggagcggaacgaac

SEQ ID NO: 89 - DNA, 47 bp, Artificial Sequence
  Synthetic construct
ccaacctaacgagcatacaacgcccaagattattatcatgtgaggcg

SEQ ID NO: 90 - DNA, 47 bp, Artificial Sequence
  Synthetic construct
ttcgaggtttcatgaggatgcaaatccaatctttatcaaaagaaaac

SEQ ID NO: 91 - DNA, 47 bp, Artificial Sequence
  Synthetic construct
agtaacagcttgagctagcaccaatagccgaacaaagttaccagaag

SEQ ID NO: 92 - DNA, 47 bp, Artificial Sequence
  Synthetic construct
tgaaaatcaactacaaagtcagagatagcccctattaccaagggatt

SEQ ID NO: 93 - DNA, 47 bp, Artificial Sequence
  Synthetic construct
gaaacgctgtctttcccagctaccttaattgtcaattacctccggct

SEQ ID NO: 94 - DNA, 47 bp, Artificial Sequence
  Synthetic construct
agtaccagaatcaatatttcggtcggtaattgcaaatgaaaactcgt

SEQ ID NO: 95 - DNA, 47 bp, Artificial Sequence
  Synthetic construct
taatcggcatacgtaataggttgggttatatatttttaacctgagca

SEQ ID NO: 96 - DNA, 47 bp, Artificial Sequence
  Synthetic construct
aaccgatttattaagaaagcgcagtctctgatgctttgaaaatggat

SEQ ID NO: 97 - DNA, 47 bp, Artificial Sequence
  Synthetic construct
cctgcctattcattatcaccaattaccgaagaaaataccgaattatc

SEQ ID NO: 98 - DNA, 46 bp, Artificial Sequence
  Synthetic construct
gtagcaagcgccgacaaaagcctaaaataaattgaggattatcaaa

SEQ ID NO: 99 - DNA, 46 bp, Artificial Sequence
  Synthetic construct
gcccacggcgaaagattcatcttctgacctcttagaatacataaat

SEQ ID NO: 100 - DNA, 46 bp, Artificial Sequence
  Synthetic construct
acagacagttgcgaatccagttacgtttagtaacaattacgctgag

SEQ ID NO: 101 - DNA, 46 bp, Artificial Sequence
  Synthetic construct
ccctcattcaggaggcaccctcagagccgccagtgaggccttgcct

SEQ ID NO: 102 - DNA, 45 bp, Artificial Sequence
  Synthetic construct
gaacaagccgcagtaagctatctgaaaccattcaccacgctacag

SEQ ID NO: 103 - DNA, 40 bp, Artificial Sequence
  Synthetic construct
tgtcgaaatccgcgacataaagtagtttatcacatcgtag

SEQ ID NO: 104 - DNA, 40 bp, Artificial Sequence
  Synthetic construct
cgccaccctcagaaccgaaccgccaccctcaggttagtaa

SEQ ID NO: 105 - DNA, 39 bp, Artificial Sequence
  Synthetic construct
aacaacatgttcagctaatgcagacgcgaaaataaattg

SEQ ID NO: 106 - DNA, 32 bp, Artificial Sequence
  Synthetic construct
ccggaataggtgtatcaccgtactttcaggga

SEQ ID NO: 107 - DNA, 32 bp, Artificial Sequence
  Synthetic construct
ataccaagacgcgcctccgacaaattctaaga

SEQ ID NO: 108 - DNA, 32 bp, Artificial Sequence
  Synthetic construct
agccaccaacataaaaggtcagttagagccgt

SEQ ID NO: 109 - DNA, 32 bp, Artificial Sequence
  Synthetic construct
tttgatgacggaaacgaaggtgaattaagact

SEQ ID NO: 110 - DNA, 32 bp, Artificial Sequence
  Synthetic construct
acgcgaggaagaaaagtggcatgattatcacc

SEQ ID NO: 111 - DNA, 32 bp, Artificial Sequence
  Synthetic construct
aaacgcaataataacggaataccctaccgcgc

SEQ ID NO: 112 - DNA, 31 bp, Artificial Sequence
  Synthetic construct
ccctttttcgttttagtttattttacaatag

SEQ ID NO: 113 - DNA, 31 bp, Artificial Sequence
  Synthetic construct
atccggtaaggtaaagtaattctgtccagac

SEQ ID NO: 114 - DNA, 31 bp, Artificial Sequence
  Synthetic construct
atttaggccagacggtgaggcaaaagaatac

SEQ ID NO: 115 - DNA, 31 bp, Artificial Sequence
  Synthetic construct
ccaataggaacccatagtatagcagagccgc

SEQ ID NO: 116 - DNA, 31 bp, Artificial Sequence
  Synthetic construct
aaatacataaaccaaccttaaataacatgta

SEQ ID NO: 117 - DNA, 30 bp, Artificial Sequence
  Synthetic construct
gaatcataaaagaactaagcagttaccatt

SEQ ID NO: 118 - DNA, 23 bp, Artificial Sequence
  Synthetic construct
cgccagcacgccagaccttgctg

SEQ ID NO: 119 - DNA, 22 bp, Artificial Sequence
  Synthetic construct
aaaatcaccagcatttgggaat

SEQ ID NO: 120 - DNA, 22 bp, Artificial Sequence
  Synthetic construct
ccgccaccctctcagagccgcc

SEQ ID NO: 121 - DNA, 22 bp, Artificial Sequence
  Synthetic construct
tgataaataagtggtttgaaat

SEQ ID NO: 122 - DNA, 22 bp, Artificial Sequence
  Synthetic construct
aaaatagcagcttttgtttaac

SEQ ID NO: 123 - DNA, 22 bp, Artificial Sequence
  Synthetic construct
atcatcgcctgcaaagtacaac

SEQ ID NO: 124 - DNA, 22 bp, Artificial Sequence
  Synthetic construct
atagaaggcttccaatagcaag

SEQ ID NO: 125 - DNA, 12 bp, Artificial Sequence
  Synthetic construct
acattctggcca

SEQ ID NO: 126 - DNA, 12 bp, Artificial Sequence
  Synthetic construct
tcagatgatggc

SEQ ID NO: 127 - DNA, 12 bp, Artificial Sequence
  Synthetic construct
catattcctgat

SEQ ID NO: 128 - DNA, 12 bp, Artificial Sequence
  Synthetic construct
tcttgacaagaa

SEQ ID NO: 129 - DNA, 12 bp, Artificial Sequence
  Synthetic construct
ggatattcatta

SEQ ID NO: 130 - DNA, 12 bp, Artificial Sequence
  Synthetic construct
cagtaataaaag

SEQ ID NO: 131 - DNA, 31 bp, Artificial Sequence
  Synthetic construct
gtcaccgatgcccgtatgtactggtaataag

SEQ ID NO: 132 - DNA, 32 bp, Artificial Sequence
  Synthetic construct
aagagaatctgctccattgacccccagcgatt

SEQ ID NO: 133 - DNA, 32 bp, Artificial Sequence
  Synthetic construct
agcaaggctacaggagtaaacagttaatgccc

SEQ ID NO: 134 - DNA, 54 bp, Artificial Sequence
  Synthetic construct
aaaaagatgtcagaagattaaattaaccagggtctgaggatccccgggtaccga

SEQ ID NO: 135 - DNA, 48 bp, Artificial Sequence
  Synthetic construct
ataaagaaaaattattgtgccggatttgttaattcttttcaccaggga

SEQ ID NO: 136 - DNA, 48 bp, Artificial Sequence
  Synthetic construct
tagaaagatgccagagcgcatcgtgtgagcgactttccaggatggtgg

SEQ ID NO: 137 - DNA, 48 bp, Artificial Sequence
  Synthetic construct
agagatagatcgccataaacaggaggcctcttccagctggacggataa

SEQ ID NO: 138 - DNA, 48 bp, Artificial Sequence
  Synthetic construct
ggctattatggcacaggccggagactagggcggtaaagcactaaatcg

SEQ ID NO: 139 - DNA, 48 bp, Artificial Sequence
  Synthetic construct
agaatacggtctttaaattgtaaatgcgcaaccgattaagttgggtaa

SEQ ID NO: 140 - DNA, 48 bp, Artificial Sequence
  Synthetic construct
gaataaggcttgccctgcagcaccgtcggtggtcaatccgccgggcgc

SEQ ID NO: 141 - DNA, 48 bp, Artificial Sequence
  Synthetic construct
atttcaactttaatcaccggacttatcaaactccagcacgatggtcat

SEQ ID NO: 142 - DNA, 48 bp, Artificial Sequence
  Synthetic construct
tctggaagtttcattctttcatttttttaaataccgcctggcgtattg

SEQ ID NO: 143 - DNA, 48 bp, Artificial Sequence
  Synthetic construct
aatgctttccggaagctttcaacgactaataggactccaacgtcaaag

SEQ ID NO: 144 - DNA, 48 bp, Artificial Sequence
  Synthetic construct
atcgtcatgagtacctaccctgtatcatacagtttggaacaagagtcc

SEQ ID NO: 145 - DNA, 47 bp, Artificial Sequence
  Synthetic construct
caggacgttgggaagagtccgtttaaaatcccactctgtcggaagca

SEQ ID NO: 146 - DNA, 47 bp, Artificial Sequence
  Synthetic construct
caccacacccgccgcgtggcgagacgttctagaatgccggaccccgg

SEQ ID NO: 147 - DNA, 47 bp, Artificial Sequence
  Synthetic construct
ctggcaagtgtagcgggcgggcgcagtcaaatctacaaagtaatcgt

SEQ ID NO: 148 - DNA, 47 bp, Artificial Sequence
  Synthetic construct
tacttctggaatatactttgtgagagatagaggccagtgccaagctt

SEQ ID NO: 149 - DNA, 47 bp, Artificial Sequence
  Synthetic construct
aaccagaaaacagttgtctggccacagtatcaaattgttcacggtca

SEQ ID NO: 150 - DNA, 47 bp, Artificial Sequence
  Synthetic construct
ttcatcaaagaaacaaaacgtacacggctggaatcagcggggtcatt

SEQ ID NO: 151 - DNA, 47 bp, Artificial Sequence
  Synthetic construct
catatcaattgcgtagggatagcgcagcaaccgggtcactgttgccc

SEQ ID NO: 152 - DNA, 47 bp, Artificial Sequence
  Synthetic construct
taaaaacccaactaattgccgccagcacatcttacactggtgtgttc

SEQ ID NO: 153 - DNA, 47 bp, Artificial Sequence
  Synthetic construct
aaaaggaaaccctcggcactccaggaacgccggcggtttgccctgag

SEQ ID NO: 154 - DNA, 47 bp, Artificial Sequence
  Synthetic construct
ccataaacgttttaactcatataggggcgcgatcagggcgatggccc

SEQ ID NO: 155 - DNA, 47 bp, Artificial Sequence
  Synthetic construct
caaatcaacgtaacaaccagcttagcgccatgcacgggacgaaaggg

SEQ ID NO: 156 - DNA, 46 bp, Artificial Sequence
  Synthetic construct
acatcgggtataatccggtgcgagattgtatcatatgtagagggta

SEQ ID NO: 157 - DNA, 46 bp, Artificial Sequence
  Synthetic construct
cctaaaacaacccttcattcaacaaggaaggccccgatttagagct

SEQ ID NO: 158 - DNA, 46 bp, Artificial Sequence
  Synthetic construct
ctattatataagagggtgtaggtgtcaataacccaaatcaagtttt

SEQ ID NO: 159 - DNA, 46 bp, Artificial Sequence
  Synthetic construct
gcgattttaagaactcggccagagcagttgggccgttttatccgct

SEQ ID NO: 160 - DNA, 46 bp, Artificial Sequence
  Synthetic construct
agaagtttttcatcagacataaatttcgtctcatcagatgccgggt

SEQ ID NO: 161 - DNA, 40 bp, Artificial Sequence
  Synthetic construct
actatcatattacgagaacagcgggtagaacgtaaaggtt

SEQ ID NO: 162 - DNA, 40 bp, Artificial Sequence
  Synthetic construct
aaatatcgtcaaaaataaccaatagccagctttcgtaatc

SEQ ID NO: 163 - DNA, 40 bp, Artificial Sequence
  Synthetic construct
cgccagggagagaatcgatgaacgggctatcaggtcattg

SEQ ID NO: 164 - DNA, 39 bp, Artificial Sequence
  Synthetic construct
cccaattctgcgaacgcgcaaatgaaagatttcttgaga

SEQ ID NO: 165 - DNA, 39 bp, Artificial Sequence
  Synthetic construct
ggattagaaaatattcattaaataaccgtgcatacgagc

SEQ ID NO: 166 - DNA, 39 bp, Artificial Sequence
  Synthetic construct
gtccacgcgaatcggccaacgcgccctgtgtgggcctca

SEQ ID NO: 167 - DNA, 34 bp, Artificial Sequence
  Synthetic construct
ataaagctaaatcggttgtatccctttaattgcg

SEQ ID NO: 168 - DNA, 32 bp, Artificial Sequence
  Synthetic construct
cgtgcctgttcttcgcgtaatgggtcagcgtg

SEQ ID NO: 169 - DNA, 32 bp, Artificial Sequence
  Synthetic construct
ggtgctgcggccagaagttaacggcgtcgctg

SEQ ID NO: 170 - DNA, 32 bp, Artificial Sequence
  Synthetic construct
catgttttaaatatgctcaattctcaaggata

SEQ ID NO: 171 - DNA, 31 bp, Artificial Sequence
  Synthetic construct
cgggcaacagctgataaccatcacctgttta

SEQ ID NO: 172 - DNA, 31 bp, Artificial Sequence
  Synthetic construct
agcaaatctgcggcgggcggttggtttgagg

SEQ ID NO: 173 - DNA, 31 bp, Artificial Sequence
  Synthetic construct
ttgcgctcaaagcctggggtgcccgcagtgt

SEQ ID NO: 174 - DNA, 31 bp, Artificial Sequence
  Synthetic construct
gtcagataataatggattcaggccgttaata

SEQ ID NO: 175 - DNA, 30 bp, Artificial Sequence
  Synthetic construct
ggacgacgttcctgtgcattaattggtttg

SEQ ID NO: 176 - DNA, 30 bp, Artificial Sequence
  Synthetic construct
tctttgctcgtcatagtttctgtaaatttc

SEQ ID NO: 177 - DNA, 27 bp, Artificial Sequence
  Synthetic construct
cttaattgctgaataccaataaaatac

SEQ ID NO: 178 - DNA, 27 bp, Artificial Sequence
  Synthetic construct
tttttcctgttttcgggaaacctgtcg

SEQ ID NO: 179 - DNA, 24 bp, Artificial Sequence
  Synthetic construct
aggcggcctgattgcctgccggtg

SEQ ID NO: 180 - DNA, 24 bp, Artificial Sequence
  Synthetic construct
tctcacgggcctcctcacagtacg

SEQ ID NO: 181 - DNA, 22 bp, Artificial Sequence
  Synthetic construct
acgagtagtaaattgctggtca

SEQ ID NO: 182 - DNA, 22 bp, Artificial Sequence
  Synthetic construct
ccgtaatgggatgggaacaaac

SEQ ID NO: 183 - DNA, 21 bp, Artificial Sequence
  Synthetic construct
ttaaacgatgctttagtgatg

SEQ ID NO: 184 - DNA, 18 bp, Artificial Sequence
  Synthetic construct
gcaaaattaagcaataaa

SEQ ID NO: 185 - DNA, 48 bp, Artificial Sequence
  Synthetic construct
tgagtgtttcggcaaaaccaaaaaccgtcggacgtccaattagtaaaa

SEQ ID NO: 186 - DNA, 32 bp, Artificial Sequence
  Synthetic construct
aggagcacgtgagtgaataaccttatcgtcgc

SEQ ID NO: 187 - DNA, 60 bp, Artificial Sequence
  Synthetic construct
cggaaattatttcggaagcgtcatacatggcttaatgcgcgtcacacgaccagtaataaa

SEQ ID NO: 188 - DNA, 59 bp, Artificial Sequence
  Synthetic construct
cctgcctattcattatcaccaattaccgaagaaaataccgaattatcatcatattcctg

SEQ ID NO: 189 - DNA, 60 bp, Artificial Sequence
  Synthetic construct
ccttattaaagccgttcgaacctcccagtaatacggattctcaagagtaatcttgacaag

SEQ ID NO: 190 - DNA, 60 bp, Artificial Sequence
  Synthetic construct
attctggccaacagagatagatcgccataaacaggaggcctcttccagctggacggataa

SEQ ID NO: 191 - DNA, 59 bp, Artificial Sequence
  Synthetic construct
agatgatggcaattcatcaaagaaacaaaacgtacacggctggaatcagcggggtcatt

SEQ ID NO: 192 - DNA, 59 bp, Artificial Sequence
  Synthetic construct
atattcattacccaaatcaacgtaacaaccagcttagcgccatgcacgggacgaaaggg

SEQ ID NO: 193 - DNA, 17 bp, Artificial Sequence
  Synthetic construct
cgaaagtgcatggggaa

SEQ ID NO: 194 - DNA, 17 bp, Artificial Sequence
  Synthetic construct
ttccccatgcactttcg

SEQ ID NO: 195 - DNA, 36 bp, Artificial Sequence
  Synthetic construct
aaaagagtctgtccatcacgcaaattaacagtgtac

SEQ ID NO: 196 - DNA, 55 bp, Artificial Sequence
  Synthetic construct
cagaaaccgttgtagcaatacttctttgaaaggaattgaggaaggttatgactgt

SEQ ID NO: 197 - DNA, 55 bp, Artificial Sequence
  Synthetic construct
atggtgctaaaatatctttaggagcacgtgagtgaataaccttgcttctaatgaa

SEQ ID NO: 198 - DNA, 68 bp, Artificial Sequence
  Synthetic construct
attaaaatgtaaatcgtcgctattaattaattttccaaatttaagcgttaaaatatattcttcaacag

SEQ ID NO: 199 - DNA, 45 bp, Artificial Sequence
  Synthetic construct
gttgaaagtcatttttgcggatgtccttttgataagagcctctct

SEQ ID NO: 200 - DNA, 44 bp, Artificial Sequence
  Synthetic construct
cacttcctggatagcgtccaattagtaaaatgtttagaattaca

SEQ ID NO: 201 - DNA, 45 bp, Artificial Sequence
  Synthetic construct
aatggaactaacggaacaacattcgttaataaaacgaacttttct

SEQ ID NO: 202 - DNA, 100 bp, Artificial Sequence
  Synthetic construct
cttttctattaaaatataatgaaaatggaaccattacagatgctgtagactgtgcacttgaccctctctcagaaacaaagtgtacgttgaaatccttcac

SEQ ID NO: 203 - DNA, 54 bp, Artificial Sequence
  Synthetic construct
gtgaaggatttcaacgtacactttgtttctgagagagggtcaagtgcacagtct

SEQ ID NO: 204 - DNA, 54 bp, Artificial Sequence
  Synthetic construct
acagcatctgtaatggttccattttcattatattttaatagaaaagtcctaggt

SEQ ID NO: 205 - DNA, 39 bp, Artificial Sequence
  Synthetic construct
aaaagagtctgtccatcacgcaaattaacaaacaaagtg

SEQ ID NO: 206 - DNA, 62 bp, Artificial Sequence
  Synthetic construct
gaccctctctcgttgtagcaatacttctttgaaaggaattgaggaaggttatggaaccatta

SEQ ID NO: 207 - DNA, 52 bp, Artificial Sequence
  Synthetic construct
atataatgaactaaaatatctttaggagcacgtgagtgaataaccttgcttc

SEQ ID NO: 208 - DNA, 61 bp, Artificial Sequence
  Synthetic construct
tgtaaatcgtcgctattaattaattttccaaatttaagcgttaaaatatattcttcaacag

SEQ ID NO: 209 - DNA, 51 bp, Artificial Sequence
  Synthetic construct
taggttgaaagtcatttttgcggatgtccttttgataagagctgtgcactt

SEQ ID NO: 210 - DNA, 52 bp, Artificial Sequence
  Synthetic construct
cagatgctgtctggatagcgtccaattagtaaaatgtttagattctattaaa

SEQ ID NO: 211 - DNA, 31 bp, Artificial Sequence
  Synthetic construct
ctaacggaacaacattcgttaataaaacgaa

SEQ ID NO: 212 - DNA, 36 bp, Artificial Sequence
  Synthetic construct
aaaagagtctgtccatcacgcaaattaacctcgagg

SEQ ID NO: 213 - DNA, 55 bp, Artificial Sequence
  Synthetic construct
ttaaattcgttgtagcaatacttctttgaaaggaattgaggaaggttatactcaa

SEQ ID NO: 214 - DNA, 55 bp, Artificial Sequence
  Synthetic construct
ccgctcctaaaatatctttaggagcacgtgagtgaataaccttgcttccaataat

SEQ ID NO: 215 - DNA, 68 bp, Artificial Sequence
  Synthetic construct
aggtttatgtaaatcgtcgctattaattaattttccaaatttaagcgttaaaatatattcttcaacag

SEQ ID NO: 216 - DNA, 45 bp, Artificial Sequence
  Synthetic construct
acaaggcgtcatttttgcggatgtccttttgataagaggaagacc

SEQ ID NO: 217 - DNA, 44 bp, Artificial Sequence
  Synthetic construct
atggcactggatagcgtccaattagtaaaatgtttagatggttc

SEQ ID NO: 218 - DNA, 45 bp, Artificial Sequence
  Synthetic construct
actgcgtctaacggaacaacattcgttaataaaacgaaggcccca

SEQ ID NO: 219 - DNA, 100 bp, Artificial Sequence; identical to SEQ ID NO: 66
  Synthetic construct

SEQ ID NO: 220 - DNA, 100 bp, Artificial Sequence
  Synthetic construct
ggccccaaggtttacccaataatactgcgtcttggttcaccgctctcactcaacatggcaaggaagaccttaaattccctcgaggacaaggcgttccaat

SEQ ID NO: 221 - DNA, 54 bp, Artificial Sequence
  Synthetic construct
attggaacgccttgtcctcgagggaatttaaggtcttccttgccatgttgagtg

SEQ ID NO: 222 - DNA, 54 bp, Artificial Sequence
  Synthetic construct
agagcggtgaaccaagacgcagtattattgggtaaaccttggggccgacgttgt

SEQ ID NO: 223 - DNA, 39 bp, Artificial Sequence
  Synthetic construct
aaaagagtctgtccatcacgcaaattaacgcttattatg

SEQ ID NO: 224 - DNA, 62 bp, Artificial Sequence
  Synthetic construct
agctggtgctcgttgtagcaatacttctttgaaaggaattgaggaaggttattggtgattct

SEQ ID NO: 225 - DNA, 52 bp, Artificial Sequence
  Synthetic construct
aaactttactctaaaatatctttaggagcacgtgagtgaataaccttgcttc

SEQ ID NO: 226 - DNA, 61 bp, Artificial Sequence; identical to SEQ ID NO: 208
  Synthetic construct

SEQ ID NO: 227 - DNA, 51 bp, Artificial Sequence
  Synthetic construct
tgggttatctgtcatttttgcggatgtccttttgataagagcaggttggac

SEQ ID NO: 228 - DNA, 52 bp, Artificial Sequence
  Synthetic construct
tcttcaggttctggatagcgtccaattagtaaaatgtttagatgctttacat

SEQ ID NO: 229 - DNA, 31 bp, Artificial Sequence; identical to SEQ ID NO: 211
  Synthetic construct

SEQ ID NO: 230 - DNA, 46 bp, Artificial Sequence
  Synthetic construct
taaagtgtactgcccggtaacaaccattatgttaattgctcctttt

SEQ ID NO: 231 - DNA, 46 bp, Artificial Sequence
  Synthetic construct
ttctccgtaggtcacgttggtgtagccgcacacaacattcgttaat

SEQ ID NO: 232 - DNA, 47 bp, Artificial Sequence
  Synthetic construct
ttccgaaagttccaggcaaggcaaagaattatgcggatggcttagag

SEQ ID NO: 233 - DNA, 46 bp, Artificial Sequence
  Synthetic construct
tacctgcagcctgtgcgtaaaaaaagatgggggggtaaactgcgga

SEQ ID NO: 234 - DNA, 47 bp, Artificial Sequence
  Synthetic construct
cactgcgcgccagcgggttccggcaaacgcgaaaatctaattacagg

SEQ ID NO: 235 - DNA, 48 bp, Artificial Sequence
  Synthetic construct
cacaattctgccagctagccagctagcctttaaaactccaacaggtca

SEQ ID NO: 236 - DNA, 48 bp, Artificial Sequence
  Synthetic construct
actattaaggcgaaaagcgggagattcatcaacattgaattttgcaaa

SEQ ID NO: 237 - DNA, 40 bp, Artificial Sequence
  Synthetic construct
cacacaacatctgccatgtacatcgttgagatataccagt

SEQ ID NO: 238 - DNA, 47 bp, Artificial Sequence
  Synthetic construct
ccccagcaagaacgtgtagtagcattaacattaatgctgtagctcaa

SEQ ID NO: 239 - DNA, 32 bp, Artificial Sequence
  Synthetic construct
accacattaaaatagcgagaggctccccctca

SEQ ID NO: 240 - DNA, 24 bp, Artificial Sequence
  Synthetic construct
gcagcctcggctcattttaggaat

SEQ ID NO: 241 - DNA, 48 bp, Artificial Sequence
  Synthetic construct
agagttgcaaccgtctagctgaaaaggtggcaaactaaagtcaaagcg

SEQ ID NO: 242 - DNA, 45 bp, Artificial Sequence
  Synthetic construct
ggcgaaaagcaagcgaaaattttaattcgccagaaaacacgacga

SEQ ID NO: 243 - DNA, 24 bp, Artificial Sequence
  Synthetic construct
tgctcattgcagatactaccttat

SEQ ID NO: 244 - DNA, 46 bp, Artificial Sequence
  Synthetic construct
agctgtttggggagaatcaaaaattagaaccttcgagcttacggtg

SEQ ID NO: 245 - DNA, 24 bp, Artificial Sequence
  Synthetic construct
ggaagatctttaccaggagaatga

SEQ ID NO: 246 - DNA, 48 bp, Artificial Sequence
  Synthetic construct
taccggggaacatcccctcataacggaacgtgttgtgaatataacgcc

SEQ ID NO: 247 - DNA, 23 bp, Artificial Sequence
  Synthetic construct
gctatatcatataacaagacttc

SEQ ID NO: 248 - DNA, 48 bp, Artificial Sequence
  Synthetic construct
actacgtgtgcccttcgcaatgcccattttttcaggtcttagagcaac

SEQ ID NO: 249 - DNA, 39 bp, Artificial Sequence
  Synthetic construct
gctcgaattccggcacacgcagagcatagtaatggttta

SEQ ID NO: 250 - DNA, 47 bp, Artificial Sequence
  Synthetic construct
ggcgccagggtggtttatcagcttgagtaataagcccgaagttgatt

SEQ ID NO: 251 - DNA, 48 bp, Artificial Sequence
  Synthetic construct
tgcggctggtccgtgaaaaaagagcgcttctgtgcacgtataccctga

SEQ ID NO: 252 - DNA, 24 bp, Artificial Sequence
  Synthetic construct
gtgctggtggcttgagaaacagaa

SEQ ID NO: 253 - DNA, 32 bp, Artificial Sequence
  Synthetic construct
ttttgttatgagaaagacaatattattgcatc

SEQ ID NO: 254 - DNA, 48 bp, Artificial Sequence
  Synthetic construct
ttggggtccctgagagcaaaagggaaattcgccaaagcgggaacctac

SEQ ID NO: 255 - DNA, 47 bp, Artificial Sequence
  Synthetic construct
gcaaacattttcccacaaagcgcgtggtgaagattttcaacaccaga

SEQ ID NO: 256 - DNA, 39 bp, Artificial Sequence
  Synthetic construct
gaggtgccaccattagatacatttagtagatttagtttg

SEQ ID NO: 257 - DNA, 46 bp, Artificial Sequence
  Synthetic construct
ggttgcgaaaacgacctttctcccattcgccaagggttatttgaat

SEQ ID NO: 258 - DNA, 47 bp, Artificial Sequence
  Synthetic construct
acgttgtgtatgagccgcaagaatgccaacggacgagaaggtttaac

SEQ ID NO: 259 - DNA, 48 bp, Artificial Sequence
  Synthetic construct
gctattttaagggagcgaagaaagcgaaaggatcacgctgaaagcgta

SEQ ID NO: 260 - DNA, 46 bp, Artificial Sequence
  Synthetic construct
gaaccctatgagagatcaccatctatttaatgcgcgaattggatta

SEQ ID NO: 261 - DNA, 47 bp, Artificial Sequence
  Synthetic construct
aaaactagtgcaaggtgttgggatcccggaaagtaacagcattcagt

SEQ ID NO: 262 - DNA, 47 bp, Artificial Sequence
  Synthetic construct
ggatgtgccatgtcaataagcaaaaatatgattgacctgcgcgtaac

SEQ ID NO: 263 - DNA, 48 bp, Artificial Sequence
  Synthetic construct
gcaggcgcggagccgctttaccagagggcgatctgattgtctgatagc

SEQ ID NO: 264 - DNA, 46 bp, Artificial Sequence
  Synthetic construct
tcagaggttttcgcactgccatcccacgcaaagctgcttacctttt

SEQ ID NO: 265 - DNA, 39 bp, Artificial Sequence
  Synthetic construct
cttctttgattagtaagcctcccagagccactttagtac

SEQ ID NO: 266 - DNA, 48 bp, Artificial Sequence
  Synthetic construct
tattaattaattttccaaatttaagcgttaaaatatattcttcaacag

SEQ ID NO: 267 - DNA, 32 bp, Artificial Sequence
  Synthetic construct
aaaagagtctgtccatcacgcaaatagcaata

SEQ ID NO: 268 - DNA, 32 bp, Artificial Sequence
  Synthetic construct
aacagttgaaaggaattgaggaagatatcttt

SEQ ID NO: 269 - DNA, 47 bp, Artificial Sequence
  Synthetic construct
caatatattaacaactaaacgattctttacagtccagacagccacca

SEQ ID NO: 270 - DNA, 48 bp, Artificial Sequence
  Synthetic construct
gagtagaacaatatctacagggaattatttatatagaaagcaaccatc

SEQ ID NO: 271 - DNA, 47 bp, Artificial Sequence
  Synthetic construct
caatagattttaatgaatcataatagttaatcagcatcggaacgagg

SEQ ID NO: 272 - DNA, 46 bp, Artificial Sequence
  Synthetic construct
acatagcgatagcttatatatttttactagaaatgacaagaacaac

SEQ ID NO: 273 - DNA, 48 bp, Artificial Sequence
  Synthetic construct
aagtgtttttataatcaccagaacggaaccagtagcaagcaacgatct

SEQ ID NO: 274 - DNA, 31 bp, Artificial Sequence
  Synthetic construct
ccctcaatgaactcaaaaatcacccaccacc

SEQ ID NO: 275 - DNA, 40 bp, Artificial Sequence
  Synthetic construct
attacctttaatacatcagccatagcgcattagttagcgt

SEQ ID NO: 276 - DNA, 48 bp, Artificial Sequence
  Synthetic construct
gtaatatccttgctgatgaacaccctaatttgaataatttcttgatac

SEQ ID NO: 277 - DNA, 48 bp, Artificial Sequence
  Synthetic construct
attagactttacatttatcatatgaagaacgcgaggctttgaggacta

SEQ ID NO: 278 - DNA, 48 bp, Artificial Sequence
  Synthetic construct
aagagtcaatagtgaagcaagacacgttatacttaaacagtttcacgt

SEQ ID NO: 279 - DNA, 46 bp, Artificial Sequence
  Synthetic construct
ttagacaggaacggtattgacaggcgtttgcacactgaggcattcc

SEQ ID NO: 280 - DNA, 47 bp, Artificial Sequence
  Synthetic construct
agcatcaccagaacaacttattagaggttgaggtgccgtcgagaggg

SEQ ID NO: 281 - DNA, 47 bp, Artificial Sequence
  Synthetic construct
aaaattaattacaaacccagagcctgaacaacgcctgtatttcgtca

SEQ ID NO: 282 - DNA, 47 bp, Artificial Sequence
  Synthetic construct
ttgcaacagccagcagagcgctaattaccaacaaaggctagcttgct

SEQ ID NO: 283 - DNA, 47 bp, Artificial Sequence
  Synthetic construct
attaaatctgatgaaaaaagccaataaatgctgaagtttccattaaa

SEQ ID NO: 284 - DNA, 47 bp, Artificial Sequence
  Synthetic construct
gtctgagagactaccactatatgcgctcaacggtttatcccaaaagg

SEQ ID NO: 285 - DNA, 47 bp, Artificial Sequence
  Synthetic construct
gggagctaaacaggaggccttgaatcggcatgaaaattcaagtttat

SEQ ID NO: 286 - DNA, 47 bp, Artificial Sequence
  Synthetic construct
gctgagaggaaaaacgcgttttctattcacatagcggggttttgctc

SEQ ID NO: 287 - DNA, 46 bp, Artificial Sequence
  Synthetic construct
aaagaagactttgccccctgaatctatcagacacggaatatatggt

SEQ ID NO: 288 - DNA, 46 bp, Artificial Sequence
  Synthetic construct
acatttttaacaccgattgagtttttgcaccttatcatccaatcaa

SEQ ID NO: 289 - DNA, 48 bp, Artificial Sequence
  Synthetic construct
aaaagttttattcattagaatcgcccaacttttgccactacgaaggca

SEQ ID NO: 290 - DNA, 47 bp, Artificial Sequence
  Synthetic construct
gaaagaggacagatgacgaactgacatatttagtagaaatccaagaa

SEQ ID NO: 291 - DNA, 48 bp, Artificial Sequence
  Synthetic construct
tataacgtgctttcctttaaagcctgcctttacaaaagggatataaaa

SEQ ID NO: 292 - DNA, 47 bp, Artificial Sequence
  Synthetic construct
gtcagtatgacgctcaatcaagttagaatggaggctgagactcctca

SEQ ID NO: 293 - DNA, 48 bp, Artificial Sequence
  Synthetic construct
aggcgaatgagtaacagttgctataagcccaagtggcaaccgacattc

SEQ ID NO: 294 - DNA, 46 bp, Artificial Sequence
  Synthetic construct
tatttacagaagatacaatgaattttgaaggtaccgcatatcccat

SEQ ID NO: 295 - DNA, 31 bp, Artificial Sequence
  Synthetic construct
acaaagaaataccaagttacaaaacagacca

SEQ ID NO: 296 - DNA, 47 bp, Artificial Sequence
  Synthetic construct
ggcgcataggctggctacgaggcgagaggcaaaaaataactcatcga

SEQ ID NO: 297 - DNA, 46 bp, Artificial Sequence
  Synthetic construct
ggcgcgtactatggtatttaccgagcaccgtgaaggtaaacgtaga

SEQ ID NO: 298 - DNA, 40 bp, Artificial Sequence
  Synthetic construct
caccagcattggcagatcgatagcttccagtaacctatta

SEQ ID NO: 299 - DNA, 47 bp, Artificial Sequence
  Synthetic construct
gctttgaaccaccaggcgggaggatagcaattgttagcaaatattga

SEQ ID NO: 300 - DNA, 100 bp, Artificial Sequence; identical to SEQ ID NO: 202
  Synthetic construct

SEQ ID NO: 301 - DNA, 100 bp, Artificial Sequence
  Synthetic construct
atcagcgaaatgcaccccgcattacgtttggtggaccctcagattcaactggcagtaaccagaatggagaacgcagtggggcgcgatcaaaacaacgtcg

SEQ ID NO: 302 - DNA, 100 bp, Artificial Sequence; identical to SEQ ID NO: 62
  Synthetic construct

SEQ ID NO: 303 - DNA, 100 bp, Artificial Sequence
  Synthetic construct
ggttgaagataacccacataataagctgcagcaccagctgtccaacctgaagaagaatcaccaggagtcaaataacttctatgtaaagcaagtaaagttt

SEQ ID NO: 304 - DNA, 100 bp, Artificial Sequence; identical to SEQ ID NO: 66
  Synthetic construct

SEQ ID NO: 305 - DNA, 100 bp, Artificial Sequence
  Synthetic construct
acgttgttttgatcgcgccccactgcgttctccattctggttactgccagttgaatctgagggtccaccaaacgtaatgcggggtgcatttcgctgattt

SEQ ID NO: 306 - DNA, 100 bp, Artificial Sequence
  Synthetic construct
gcgtagaagccttttggcaatgttgttccttgaggaagttgtagcacgattgcagcattgttagcaggattgcgggtgccaatgtgatcttttggtgtat

SEQ ID NO: 307 - DNA, 100 bp, Artificial Sequence
  Synthetic construct
agtctgacctcgcgcggagccccgttctctgggaactcacctccccgaagctcagggagagccctgttagggccgcctctggccctagtctcagaccttc

SEQ ID NO: 308 - DNA, 100 bp, Artificial Sequence
  Synthetic construct
atcttcaagtctctgtgcgatctcggctttgagggggcctgacgggacgatagagagaacgtacgtttcgacctcggttagaagactcatctttcaatat

SEQ ID NO: 309 - DNA, 100 bp, Artificial Sequence
  Synthetic construct
aaatcagcgaaatgcaccccgcattacgtttggtggaccctcagattcaactggcagtaaccagaatggagaacgcagtggggcgcgatcaaaacaacgt

SEQ ID NO: 310 - DNA, 10 bp, Artificial Sequence
  Synthetic construct
gcgatcaaaa

SEQ ID NO: 311 - DNA, 10 bp, Artificial Sequence
  Synthetic construct
tggcagtaac

SEQ ID NO: 312 - DNA, 10 bp, Artificial Sequence
  Synthetic construct
cctcagattc

SEQ ID NO: 313 - DNA, 10 bp, Artificial Sequence
  Synthetic construct
agcgaaatgc

SEQ ID NO: 314 - DNA, 10 bp, Artificial Sequence
  Synthetic construct
gcagtggggc

SEQ ID NO: 315 - DNA, 10 bp, Artificial Sequence
  Synthetic construct
cagaatggag

SEQ ID NO: 316 - DNA, 10 bp, Artificial Sequence
  Synthetic construct
tttggtggac

SEQ ID NO: 317 - DNA, 10 bp, Artificial Sequence
  Synthetic construct
accccgcatt

SEQ ID NO: 318 - DNA, 6 bp, Artificial Sequence
  Synthetic construct
acgttg